(19) United States
(12) Patent Application Publication
(10) Pub. No.: US 2026/0335384 A1
(43) Pub. Date: Oct. 29, 2026

(54) COMPOSITIONS AND METHODS FOR THE TREATMENT OF SKIN DISEASES AND CONDITIONS INCLUDING ECZEMA

(71) Applicant: PHI THERAPEUTICS, INC., San Francisco, CA (US)

(72) Inventors: Yug VARMA, San Francisco, CA (US); Elijah HORWITZ, San Francisco, CA (US)

(21) Appl. No.: 19/575,210

(22) Filed: Mar. 23, 2026

Related U.S. Application Data
(63) Continuation of application No. 19/003,326, filed on Dec. 27, 2024, now Pat. No. 12,605,416.
Application No. 19/003,326 is a continuation of application No. PCT/US2024/022657, filed on Apr. 2, 2024.
(60) Provisional application No. 63/493,834, filed on Apr. 3, 2023.

(51) Int. Cl. A61K 35/76; A61K 9/00; A61K 9/10; A61K 36/899; A61K 38/47; A61P 17/00; A61P 29/00; A61P 31/02

(52) U.S. Cl. CPC A61K 35/76; A61K 9/0014; A61K 9/10; A61K 36/899; A61K 38/47; A61P 17/00; A61P 29/00; A61P 31/02

(57) ABSTRACT

Disclosed herein are therapeutic compositions comprising bacteriophage and colloidal oatmeal, and methods of using the compositions for the treatment of skin diseases and conditions including eczema.

### CROSS-REFERENCE TO RELATED APPLICATIONS

This application is a continuation of U.S. application Ser. No. 19/003,326 filed Dec. 27, 2024, which is a continuation of International Patent Application No. PCT/US2024/022657 filed Apr. 2, 2024, and which claims priority to U.S. Provisional Appl. No. 63/493,834, filed Apr. 3, 2023, the contents of each of which is herein incorporated by reference in their entirety.

### SEQUENCE LISTING

A Sequence Listing accompanies this application and is submitted as an xml file of the sequence listing named “177601.00023. xml” which is 264,979 bytes in size and was created on Jun. 30, 2026. The sequence listing is electronically submitted with the application and is incorporated herein by reference in its entirety.

### FIELD

The field of the invention relates to therapeutic compositions comprising bacteriophage and colloidal oatmeal, and methods of using the compositions for the treatment of skin diseases and conditions including eczema.

### BACKGROUND

Eczema is a common skin condition characterized by red, itchy skin and small blisters also known in the art as skin rash or rash or dermatitis. There is no cure for eczema, but there are many treatments, ranging from special diets to emollients and immunosuppressive ointments like e.g. corticosteroid ointment. While corticosteroids, such as hydrocortisone or clobetasol propionate (topical, oral or intradermal administration) usually bring about improvements, they also may have side effects. Prolonged use of topical corticosteroids is thought to increase the risk of side effects, the most common of which is the skin becoming thin and fragile (atrophy). Because of this, if used on the face or other delicate skin, a low-strength steroid should be used or applied less frequently. Additionally, high-strength steroids used over large areas, or under occlusion, may be absorbed into the body, causing hypothalamic-pituitary-adrenal axis suppression (HPA axis suppression).

Due to the impaired skin barrier in atopic dermatitis an increase in skin infections with bacteria such as Staphylococcus aureus or fungi might be the result. For more severe cases, dermatologists may also prescribe either topical or oral conventional antibiotics such as penicillin, streptomycin and chloramphenicol. The antibiotics prevent infection that can result from impaired skin barrier such as cracked skin. S. aureus colonization or infection is the most common cause of increased eczema severity. The effectiveness of antibiotic treatments varies from person to person. The well-known disadvantages of conventional antibiotics are specificity, i.e. also non-pathogenic and/or beneficial bacteria are killed, and the risk of developing resistance, not only by the target bacterial cells but possibly also by other pathogenic bacteria. Furthermore, conventional, systemic antibiotic treatment can interact with other drugs, including contraceptive pills. Certain antibiotics cannot be combined with the use of alcohol. Accordingly, there is a need for improved treatment of eczema.

### SUMMARY

Disclosed herein are compositions, kits, and methods for the prevention and treatment of skin disease and disorders, such as, but not limited to eczema.

In some embodiment, compositions disclosed herein are formulated for topical administration and comprise bacteriophage, and colloidal oatmeal. In some embodiments, the bacteriophage comprises a Staphylococcus bacteriophage. In some embodiments, the bacteriophage comprises or consists of one or more Staphylococcus aureus (S. aureus) bacteriophage. In some embodiments, the S. aureus bacteriophage comprises or consists of one or more of SEQ ID NO: 1 (MESA-01), SEQ ID NO: 44 (MESA-05), SEQ ID NO: 45 (MESA-11), and/or SEQ ID NO: 46 (SaGU1).

In some embodiments, the composition further comprises one or more additional active agents. By way of example, but not by way of limitation, the one or more additional active agents comprise a ceramide, hyaluronic acid, glycerin, petrolatum, niacinamide, an enzyme, and/or a probiotic bacteria.

In some embodiments, the composition comprises one or more enzymes, such as, but not limited to a bacterial cell wall degrading enzyme, an endolysin, and/or an anti-aging enzyme.

In some embodiments, the compositions disclosed herein consist of one or more S. aureus bacteriophage and colloidal oatmeal. The bacteriophage are free in the composition and are not linked to a solid support such as a bead. In some embodiments, the page are encapsulated, or occluded in suspension in the medium.

In some embodiments, the S. aureus bacteriophage comprises or consists of SEQ ID NO: 1, SEQ ID NO: 44, SEQ ID NO: 45, and/or SEQ ID NO: 46. In some embodiments, the S. aureus bacteriophage comprises a sequence that is at least 90%, 91%, 92%, 93%, 94%, 95%, 96%, 97%, 98%, or 99% identical to one of SEQ ID NOs: 1, 44, 45, and/or 46.

In some embodiments, the bacteriophage comprises a nucleic acid sequences at least 90% identical to one or more of SEQ ID NOs: 1, 2, 4, 6, 8, 10, 12, 14, 16, 18, 20, 22, 24, 26, 28, 30, 32, 34, 36, 38, 40, 42, 44, 45, and/or 46. In some embodiments, the bacteriophage comprises a nucleic acid that encodes a polypeptide at least 90% identical to one or more of SEQ ID NOs: 3, 5, 7, 9, 11, 13, 15, 17, 19, 21, 23, 25, 27, 29, 31, 33, 35, 37, 39, 41, and/or 43.

Also disclosed herein are methods of treating skin diseases or disorders, e.g., eczema, in a subject in need thereof. In some embodiments, the methods comprise topically administering a composition of any of the embodiments described above to a region of the subject's skin comprising the skin disease or disorder, e.g., eczema. In some embodiments, administration comprises once daily, twice daily, three times daily or four times daily administration. In some embodiments, the composition is administered for one week, two weeks, three weeks or more. In some embodiments, the treatment reduces one or more of the following eczema symptoms: itchy skin, dry skin, redness, and swelling of the region of the subject's skin administered the composition. In some embodiments, reducing the one or more eczema symptoms occurs more quickly in treated subject than in a control subject (e.g., a subject not treated with a composition as described herein, e.g., including one or more S. aureus bacteriophage).

For example, in some embodiments, the treated subject exhibits improvement 5%, 10%, 15%, 20%, 25%, 30%, 35%, 40%, 45%, or 50% faster than a comparable control subject. In some embodiments, the treated subject exhibits at least about 5%, 10%, 15%, 20%, 25%, 30%, 35%, 40%, 45%, 50% or greater improvement in at least one symptom as compared to a comparable control subject, in the same amount of time.

In some embodiments, the eczema is selected from contact eczema, allergic contact eczema, seborrheic eczema, nummular eczema, neurodermatitis, stasis dermatitis, dyshidrotic eczema. In some embodiments, the eczema comprises atopic eczema.

In some embodiments, the subject to be treated is human.

### BRIEF DESCRIPTION OF THE DRAWINGS

Non-limiting embodiments of the presently systems, methods, and kits may be described by way of example with reference to the accompanying figures.

FIG. 1A, FIG. 1B, FIG. 1C. An annotated schematic of an exemplary full-length bacteriophage DNA sequence (SEQ ID NO: 1). Twenty-one segments are shown with the following SEQ ID NOs: 2 (CDS 0 hypothetical protein), 4 (CDS 1 hypothetical protein), 6 (CDS 2 major head protein), 8 (CDS 3 upper collar connector), 10 (CDS 4 adaptor Ad4), 12 (CDS 5 minor tail protein), 14 (CDS 6 endolysin), 16 (CDS 7 tail protein), 18 (CDS 8 tail protein), 20 (CDS 9 holin), 22 (CDS 10 hypothetical protein), 24 (CDS 11 DNA polymerase), 26 (CDS 12 terminase), 28 (CDS 13 hypothetical protein), 30 (CDS 14 hypothetical protein), 32 (CDS 15 hypothetical protein), 34 (CDS 16 hypothetical protein), 36 (CDS 17 hypothetical protein), 38 (CDS 18 hypothetical protein), 40 (CDS 19 hypothetical protein), and 42 (CDS 20 hypothetical protein). These sequence segments can be transcribed and translated into the representative polypeptides of SEQ ID NOs: 3, 5, 7, 9, 11, 13, 15, 17, 19, 21, 23, 25, 27, 29, 31, 33, 35, 37, 39, 41, and 43, respectively.

FIG. 2 is a graph showing S. aureus bacteriophage is more stable in the presence of 1% colloidal oatmeal (CO). S. aureus bacteriophage were incubated at 30 °C for 48 hours with and without 1% CO. Without CO: 0.53×10⁹ pfu/mL. With 1% CO: 1.4×10⁹ pfu/mL. Thus, 1% CO confers 2.64× (264%) higher stability to S. aureus bacteriophage.

FIG. 3 is a graph showing S. aureus bacteria survival is lower in the present of 1% colloidal oatmeal. Bacteriophage killing of S. aureus bacteria was measured with our without 1% CO. Lower S. aureus bacterial survival indicates better killing. S. aureus survival was lower in the presence of 1% CO. No CO: 1.23×10¹⁰ cfu/mL. With 1% CO: 0.73×10¹⁰ cfu/mL. Thus, CO enhances the bacteriophage killing of S. aureus bacteria.

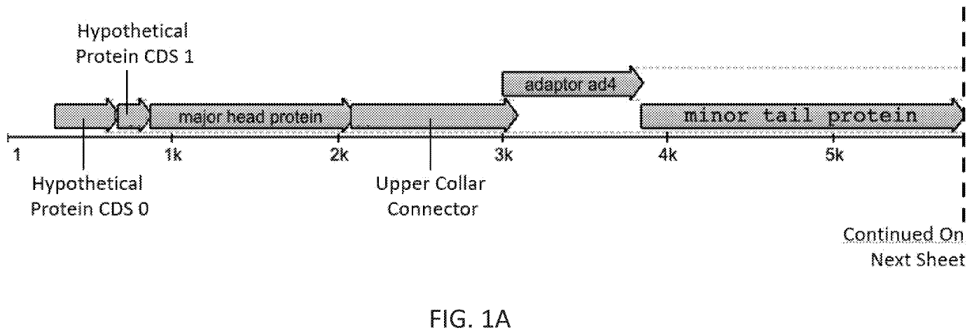

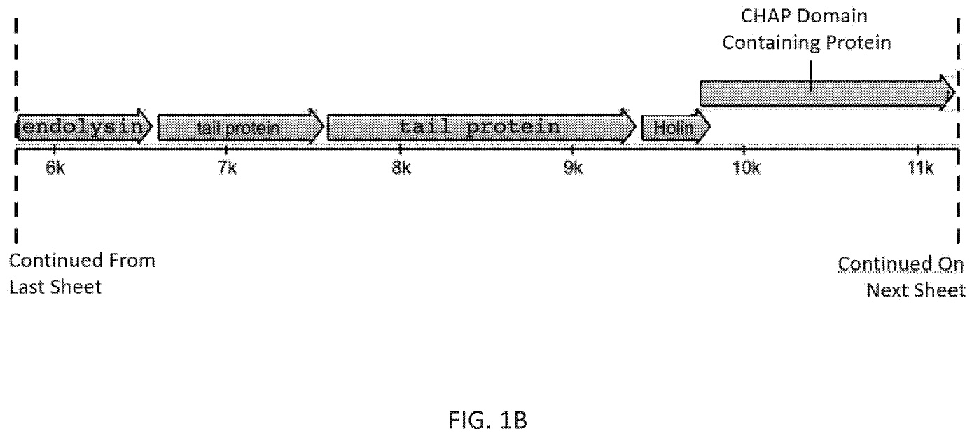

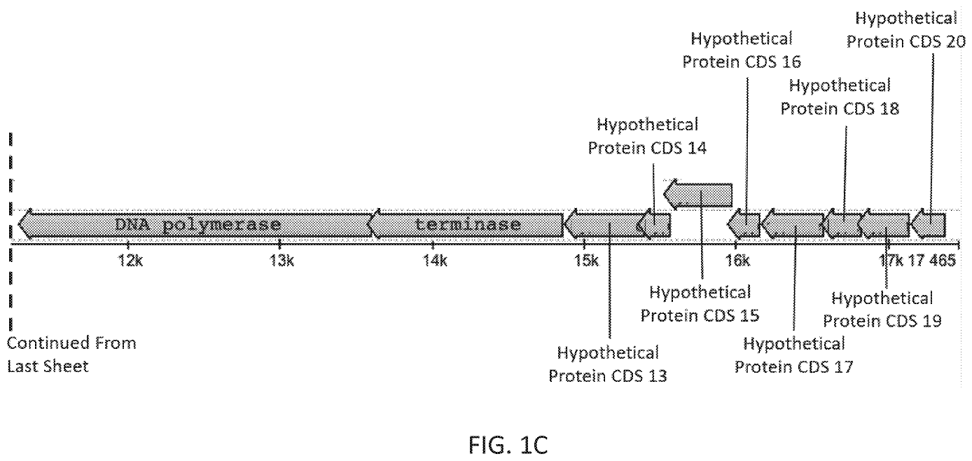

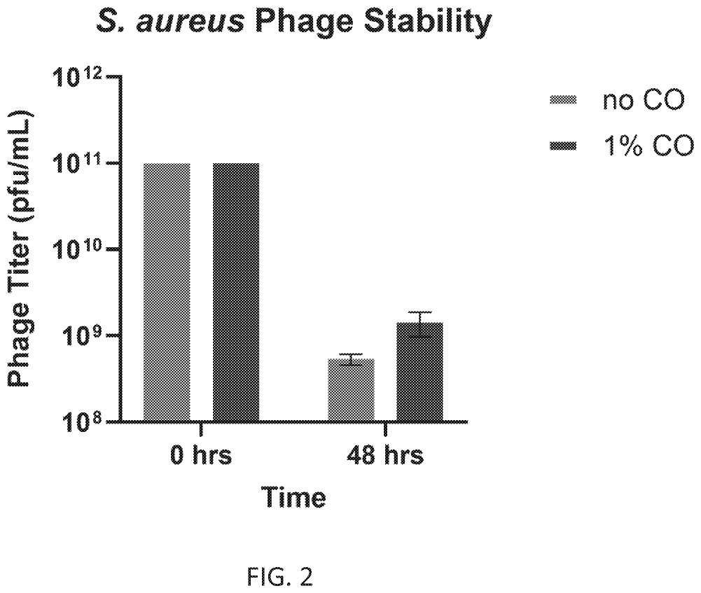

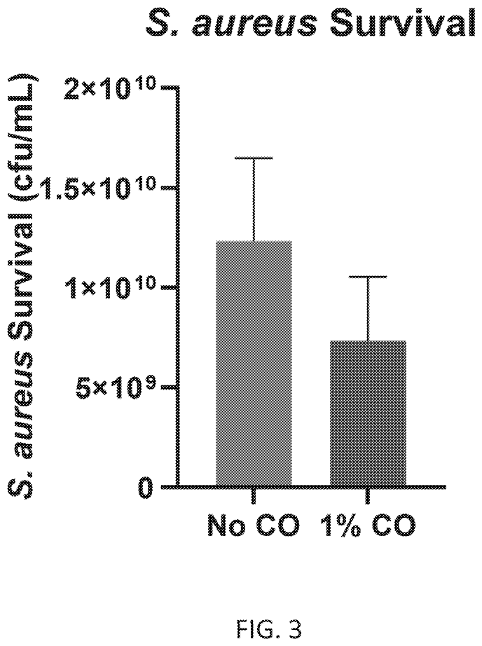

### DETAILED DESCRIPTION

Disclosed herein are compositions and methods for the treatment of skin disease or conditions including, but not limited to eczema. The disclosed compositions comprise bacteriophage and colloidal oatmeal and in some embodiments, such compositions are topically administered to a subject suffering from a skin disease or condition, such as, for example, eczema.

In some embodiments, the method of treatment is a method for preventing, delaying and/or curing a skin disease or condition, such as, but not limited to eczema, acne, rosacea, pimples, impetigo, boils, furuncles, cellulitis folliculitis, hidradenitis suppurativa, psoriasis, carbuncles, scaled skin syndrome, and abscesses. Preferably, said method of treatment is a method for preventing, delaying, treating, and/or curing eczema such as atopic dermatitis, allergic contact eczema, contact eczema, dyshidrotic eczema, neurodermatitis, nummular eczema, seborrheic eczema, stasis dermatitis, preferably atopic dermatitis. In some embodiments, the method of treatment is topical treatment of eczema.

To aid in understanding the invention, several terms are defined throughout the specification.

Unless defined otherwise, all technical and scientific terms used herein have the same meaning as commonly understood by one of skill in the art. Although any methods and materials similar to or equivalent to those described herein can be used in the practice or testing of the claims, the exemplary methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

The term “about” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” will depend upon the particular system under study.

The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. Polynucleotides are polymers of any length, including longer lengths, e.g., 200, 300, 500, 1000, 2000, 3000, 5000, 7000, 10000, 20000, 30000, 40000 etc. Polynucleotides and oligonucleotides will generally contain phosphodiester bonds, although in some cases, nucleic acid analogs are included that may have alternate backbones, that include, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

The term “bp” and the like refer, in the usual and customary sense, to the indicated number of base pairs.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire nucleic acid or polypeptide sequence or individual portions or domains of a nucleic acid or polypeptide), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. In embodiments, the identity exists over a region that is about or at least about 20, 50, 100, 1000, 2500, 5000, 7500, 10000, 15000, 20000, 25000, or 30000 amino acids or nucleotides in length to about, less than about, or at least about 31000, 32000, 33000, 34000 or 35000 amino acids or nucleotides in length. Optionally, the identity exists over a region that is at least about 10 to about 100, about 20 to about 75, about 30 to about 50 amino acids or nucleotides in length. Optionally, the identity exists over a region that is at least about 50 amino acids in length, or more preferably over a region that is 100 to 500 or 1000 or more amino acids in length. Included herein are phages comprising nucleic acids (e.g., a genome or a portion thereof) having sequences that are substantially identical to any of SEQ ID NOs: 1, 2, 4, 6, 8, 10, 12, 14, 16, 18, 20, 22, 24, 26, 28, 30, 32, 34, 36, 38, 40, 42, 44, 45, and/or 46. Non-limiting examples of phages provided herein comprise genomes having sequences that are substantially identical to SEQ ID NO: 1, 44, 45, and/or 46. In addition or alternatively, included herein are phages comprising polynucleotides encoding polypeptides having sequences that are substantially identical to any of SEQ ID NOs: 3, 5, 7, 9, 11, 13, 15, 17, 19, 21, 23, 25, 27, 29, 31, 33, 35, 37, 39, 41, and 43.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions in which a sequence may be compared to a reference sequence of the same number of contiguous positions after the two sequences are optimally aligned. In embodiments, a comparison window includes about or at least about 20, 50, 100, 1000, 2500, 5000, 7500, 10000, 15000, 20000, 25000, or 30000 to about, less than about, or at least about 31000, 32000, 33000, 34000 or 35000 contiguous positions. In embodiments, the comparison window is the entire length of a reference sequence, such as the sequence of a bacteriophage genome. Methods of alignment of sequences for comparison are well-known in the art. In embodiments, optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An example of algorithms suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. As will be appreciated by one of skill in the art, the software for performing BLAST analyses is publicly available through the website of the National Center for Biotechnology Information (NCBI). In embodiments, BLAST and BLAST 2.0 are used, with the parameters described herein, to determine percent sequence identity for the nucleic acids and proteins.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions to a peptide, polypeptide, or protein sequence which alters a single amino acid is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

As used herein a “Staphylococcus aureus (S. aureus) bacteriophage” is a bacteriophage that infects, replicates within, and kills S. aureus cells. In some embodiments, an S. aureus bacteriophage is a lytic S. aureus bacteriophage. In embodiments, an S. aureus bacteriophage is capable of lysing an S. aureus bacterium and incapable of lysing any bacterium which is not S. aureus. In embodiments, an S. aureus bacteriophage is incapable of sustaining lysogeny in a bacterium. In embodiments, the use of a bacteriophage that can lyse S. aureus but is incapable of sustaining lysogeny has the advantage that the bacteriophage cannot lie dormant within a bacterium, but must lyse the bacterium and hence kill it. In embodiments, an S. aureus bacteriophage lacks the ability to express at least one gene necessary for sustaining lysogeny. The term “lacks the ability to express at least one gene necessary for sustaining lysogeny” is intended to indicate that the S. aureus bacteriophage lacks the ability to produce a fully functional protein product necessary to sustain lysogeny, for example, as the result of one or more point mutations or full or partial deletions of the genome. In embodiments, the S. aureus bacteriophage has a genome that lacks all or part of at least one gene necessary for sustaining lysogeny (e.g., artificially or naturally, e.g., the strain is or is derived from a strain that lacks all or part of at least one gene necessary for sustaining lysogeny). In embodiments, the S. aureus bacteriophage may comprise modification (e.g. mutations, insertions or deletions) in the genome in non-coding regions that may, nonetheless, affect the ability of the phage to sustain lysogeny, for example defects in the genome integration site(s) (e.g. a/att/site) or in a repressor binding site. In embodiments, an S. aureus bacteriophage is naturally occurring and isolated, with the added advantage that artificial mutations need not be introduced into the bacteriophage. In embodiments, an S. aureus bacteriophage is capable of lysing a plurality of strains of S. aureus bacterium. In embodiments, an S. aureus bacteriophage is capable of lysing at least about 5, 10, 15, 20, 25, 30 or more strains of the S. aureus bacterium. Non-limiting examples of S. aureus bacteriophages are disclosed herein and include, without limitation those described in References 1-9, incorporated herein by reference in their entireties.

In embodiments, the S. aureus bacteriophage has a genome having sequence identity of at least about 80%, 81%, 82%, 83%, 84%, 85%, 86%, 87%, 88%, 89%, 90%, 95%, 99%, 99.1%, 99.2%, 99.3%, 99.4%, 99.5%, 99.6%, 99.7%, 99.8%, 99.9% identity with SEQ ID NO: 1, SEQ ID NO: 44, SEQ ID NO: 45, or SEQ ID NO: 46. In some embodiments, the S. aureus bacteriophage has a genome comprising the sequence of SEQ ID NO: 1, SEQ ID NO: 44, SEQ ID NO: 45, or SEQ ID NO: 46 or consisting of the sequence of SEQ ID NO: 1, SEQ ID NO: 44, SEQ ID NO: 45, or SEQ ID NO: 46. In embodiments, the genome of the S. aureus bacteriophage has no insertions or deletions compared to SEQ ID NO: 1, SEQ ID NO: 44, SEQ ID NO: 45, or SEQ ID NO: 46. In embodiments, the genome of the S. aureus bacteriophage has no insertions or deletions, and only conservative substitutions compared to SEQ ID NO: 1, SEQ ID NO: 44, SEQ ID NO: 45, or SEQ ID NO: 46.

Additional non-limiting examples of S. aureus bacteriophage, well known in the art, are provided in References 5 and 11-13, which are incorporated herein by reference in their entireties and include without limitation the following bacteriophages (GenBank/NCBI Accession Number in parenthesis): vB_SauM-A (No. MN539736), vB_SauM-C(No. MN539737), vB_SauM-D (No. MN539738), JD007 (No. JX878671), MCE-2024 (No. KJ888149), phiIPLA-RODI (No. KP027446), SaGU1 (No. LC574321), Stau2 (No. NC_030933), IME-SA1 (No. NC_047729), PT1028 (No. AY954948), 66 (No. AY954949), 44AHJD (No. NC_004678), P68 (No. NC_004679), 187 (No. AY954950), 69 (No. AY954951), 53 (No. AY954952), 85 (No. AY954953), 2638A (No. AY954954), 77 (No. AY508486), 42e (No. AY954955), 3A (No. AY954956), 47 (No. AY954957), 37 (No. AY954958), EW (No. AY954959), 96 (No. AY954960), ROSA (No. AY954961), 71 (No. AY954962), 55 (No. AY954963), 29 (No. AY954964), 52A (No. AY954965), 88 (No. AY954966), 92 (No. AY954967), X2 (No. AY954968), K (No. AY176327), G1 (No. AY954969), Twort (No. AY954970), PG-2021_1 (No. MZ417323), PG-2021_3, PG-2021_4 (No. MZ417338), PG-2021_7, PG-2021_8 (No. MZ417350), PG-2021_9 (No. MZ417354), PG-2021_10 (No. MZ417315), PG-2021_11, PG-2021_12 (No. MZ417316), PG-2021_13, PG-2021_14 (No. MZ417317), PG-2021_15 (No. MZ417318), PG-2021_16 (No. MZ417319), PG-2021_17 (No. MZ417320), PG-2021_18 (No. MZ417321), PG-2021_22 (No. MZ417324), PG-2021_23 (No. MZ417325), PG-2021_24, PG-2021_27 (No. MZ417326), PG-2021_28, PG-2021_33 (No. MZ417330), PG-2021_34, PG-2021_37, PG-2021_38 (No. MZ417332), PG-2021_39, PG-2021_40 (No. MZ417333), PG-2021_42, PG-2021 43 (No. MZ417335), PG-2021_45, PG-2021_47 (No. MZ417337), PG-2021_48, PG-2021_49, PG-2021_50, PG-2021_51, PG-2021_52, PG-2021_53, PG-2021_63, PG-2021_64 (No. MZ417340), PG-2021_68 (No. MZ417342), PG-2021_78, PG-2021_84 (No. MZ417345), PG-2021_86 (No. MZ417346), and PG-2021_87 (No. MZ417347). In embodiments, the S. aureus bacteriophage has a genome having sequence identity of at least about 80%, 81%, 82%, 83%, 84%, 85%, 86%, 87%, 88%, 89%, 90%, 95%, 99%, 99.1%, 99.2%, 99.3%, 99.4%, 99.5%, 99.6%, 99.7%, 99.8%, 99.9% identity with one of the above-referenced phage. The terms “phage” and “bacteriophage” are used interchangeably herein.

Staphylococcus aureus (S. aureus) is a Gram-positive spherically shaped bacterium and is a common member of the microbiota of the body, frequently found on the skin. While typically commensal, it can also be pathogenic, causing skin infections and/or exacerbating diseases and conditions affecting the skin. S. aureus strains are known in the art, and a non-limiting example of a well-known exemplary S. aureus bacteria strain UAMS-1, is described in Reference 10, which is incorporated herein by reference in its entirety.

The term “isolated,” when applied to a bacterium or bacteriophage, refers to a bacterium or bacteriophage that has been (1) separated from at least some of the components with which it was associated when initially produced (whether in nature or in an experimental setting), and/or (2) produced, prepared, purified, and/or manufactured by the hand of man, e.g. using artificial culture conditions such as (but not limited to) growing on a plate and/or in a fermenter. Isolated bacteria include those bacteria that are cultured, even if such cultures are not monocultures. In embodiments, the isolated bacteria are bacteria that are cultured as a monoculture (e.g., on a plate or in liquid culture such as in a fermenter). Isolated bacteria and bacteriophages may be separated from at least about 10%, about 20%, about 30%, about 40%, about 50%, about 60%, about 70%, about 80%, about 90%, about 95%, about 96%, about 97%, about 99% or more of the other components with which they were initially associated (e.g., by weight). In embodiments, isolated bacteria are more than about 80%, about 85%, about 90%, about 91%, about 92%, about 93%, about 94%, about 95%, about 96%, about 97%, about 98%, about 99%, or more than about 99% pure (e.g., by weight). In embodiments, isolated bacteriophages are more than about 80%, about 85%, about 90%, about 91%, about 92%, about 93%, about 94%, about 95%, about 96%, about 97%, about 98%, about 99%, or more than about 99% pure (e.g., by weight). In embodiments, a composition provided herein includes one or more isolated bacteriophages. In embodiments, a composition provided herein includes an isolated bacteriophage. In embodiments, a bacteriophage that is administered is an isolated bacteriophage. In embodiments, a composition provided herein includes one or more isolated bacteria. In embodiments, a composition provided herein includes an isolated bacterium. In embodiments, a bacterium that is administered is an isolated bacterium.

The bacteriophage disclosed herein (e.g., S. aureus bacteriophage) are active agents. In some embodiments, the bacteriophage active agent consists of bacteriophage (i.e., bacteriophage alone, e.g., not linked to a solid support; bacteriophage that are free in solution). In other embodiments, a bacteriophage active agent may comprise or consist of bacteriophage linked to another component, such as a bead or other molecule. In still other embodiments, a bacteriophage active agent may include both free bacteriophage and bacteriophage linked to another molecule, such as a solid support. In some embodiments, phage may be encapsulated or occluded in suspension; such phage may be free or linked to a solid support.

A “control” sample or value refers to a sample that serves as a reference, usually a known reference, for comparison to a test sample. For example, a test sample can be taken from a test condition, e.g., in the presence of a test compound (e.g., enzyme) or phage, and compared to samples from known conditions, e.g., in the absence of the test compound, phage, or bacterium (negative control), or in the presence of a known compound, phage, or bacterium (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life, the degradation of a biofilm or a component thereof, or bacterial cell lysis, or symptoms of a disease, condition, infection, etc.) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. A control may also comprise, for example, a subject or a subject population that is suffering from or afflicted with the same disease, symptoms, or condition, etc. as a test subject or a test population, but receives a placebo, partial treatment, or no treatment.

The term “disease” refers to any deviation from the normal health of a mammal and includes a state when disease symptoms are present, as well as conditions in which a deviation (e.g., dysbiosis, infection, gene mutation, genetic defect, etc.) has occurred, but symptoms are not yet manifested. In embodiments, the disease is eczema. In embodiments, the disease includes dermal dysbiosis. In embodiments, methods, compositions, systems, phages, and probiotic bacteria provided herein are suitable for use in a subject that is a member of the Vertebrate class, Mammalia, including, without limitation, primates (such as humans), livestock, work animals, and domestic pets (e.g., a companion animal). In embodiments, a subject is a human subject. As used herein, a “symptom” of a disease includes any clinical or laboratory manifestation associated with the disease, and is not limited to what a subject can feel or observe.

As used herein, the term “dermal dysbiosis” means a difference in the skin microbiota compared to a healthy or general population. In embodiments, the dysbiosis is on the surface of the skin, within skin (e.g., within a skin region or layer of skin cells), within a gland, and/or within a pore of the skin. In embodiments, the dysbiosis is within sweat and/or sebum. In embodiments, the skin is on the face (e.g., the forehead, one or more cheeks, the nose, or the chin of a subject). In embodiments, the skin is anywhere on the body, including but not limited to on the shoulders, chest, arms, elbows, legs, and back. In embodiments, dermal dysbiosis includes a change in microbiota commensal species diversity as compared to a healthy or general population and may include decrease of beneficial microorganisms and/or increase of pathobionts (pathogenic or potentially pathogenic microorganisms) and/or decrease of overall microbiota species diversity. Many factors can lead to dysbiosis, including hormonal changes, infrequent washing, cosmetic use, antibiotic use, psychological and physical stress, radiation, and dietary changes.

As used herein, “eczema,” is a general term for many types of skin inflammation, also known as dermatitis. The most common form of eczema is atopic eczema or dermatitis (many practitioners use the terms eczema and dermatitis interchangeably). However, there are many other different forms of eczema including, contact eczema, allergic contact eczema, seborrheic eczema, nummular eczema, neurodermatitis, stasis dermatitis, dyshidrotic eczema, among others.

Atopic dermatitis is a chronic skin disease characterized by itchy, inflamed skin and is the most common cause of eczema. The condition tends to come and go, depending upon exposures to triggers or causative factors. Factors that may cause atopic dermatitis (allergens) include environmental factors like molds, pollen, or pollutants; contact irritants like soaps, detergents, nickel (present in jewelry), or perfumes; food allergies; or other allergies. Around two-thirds of those who develop the condition do so prior to one year of age. When the disease starts in infancy, it is sometimes termed infantile eczema.

Contact eczema (i.e., contact dermatitis) is a localized reaction that includes redness, itching, and burning in areas where the skin has come into contact with an allergen (an allergy-causing substance to which an individual is sensitized) or with a general irritant such as an acid, a cleaning agent, or other chemical. Other examples of contact eczema include reactions to laundry detergents, soaps, nickel (present in jewelry), cosmetics, fabrics, clothing, and perfume. Due to the vast number of substances with which individuals have contact, it can be difficult to determine the trigger for contact dermatitis. The condition is sometimes referred to as allergic contact eczema (i.e., allergic contact dermatitis) if the trigger is an allergen, and irritant contact eczema (i.e., irritant contact dermatitis) if the trigger is an irritant. Skin reactions to poison ivy, oak and/or sumac are examples of allergic contact eczema. People who have a history of allergies have an increased risk for developing contact eczema.

Seborrheic eczema (i.e., seborrheic dermatitis) is a form of skin inflammation of unknown cause. The signs and symptoms of seborrheic eczema include yellowish, oily, scaly patches of skin on the scalp, face, and occasionally other parts of the body. Dandruff and “cradle cap” in infants are examples of seborrheic eczema. It is commonplace for seborrheic dermatitis to inflame the face at the creases of the cheeks and/or the nasal folds. Seborrheic dermatitis is not necessarily associated with itching. This condition tends to run in families. Emotional stress, oily skin, infrequent shampooing, and weather conditions may all increase a person's risk of developing seborrheic eczema. One type of seborrheic eczema is also common in people with AIDS.

Nummular eczema (i.e., nummular dermatitis) is characterized by coin-shaped patches of irritated skin—most commonly located on the arms, back, buttocks, and lower legs—that may be crusted, scaling, and extremely itchy. This form of eczema is relatively uncommon and occurs most frequently in elderly men. Nummular eczema is usually a chronic condition. A personal or family history of atopic dermatitis, asthma, or allergies increases the risk of developing the condition.

Neurodermatitis, also known as lichen simplex chronicus, is a chronic skin inflammation caused by a scratch-itch cycle that begins with a localized itch (e.g., an insect bite) that becomes intensely irritated when scratched. Women are more commonly affected by neurodermatitis than men, and the condition is most frequent in people 20-50 years of age. This form of eczema results in scaly patches of skin on the head, lower legs, wrists, or forearms. Over time, the skin can become thickened and leathery. Stress can exacerbate the symptoms of neurodermatitis.

Stasis dermatitis is a skin irritation on the lower legs, generally related to the circulatory problem known as venous insufficiency, in which the function of the valves within the veins has been compromised. Stasis dermatitis occurs almost exclusively in middle-aged and elderly people, with approximately 6%-7% of the population over 50 years of age being affected by the condition. The risk of developing stasis dermatitis increases with advancing age. Symptoms include itching and/or reddish-brown discoloration of the skin on one or both legs. Progression of the condition can lead to the blistering, oozing skin lesions seen with other forms of eczema, and ulcers may develop in affected areas. The chronic circulatory problems lead to an increase in fluid buildup or edema in the legs. Stasis dermatitis has also been referred to as varicose eczema.

Dyshidrotic eczema (i.e., dyshidrotic dermatitis) is an irritation of the skin on the palms of hands and soles of the feet characterized by clear, deep blisters that itch and burn. The cause of dyshidrotic eczema is unknown. Dyshidrotic eczema is also known as vesicular palmoplantar dermatitis, dyshidrosis, or pompholyx. This form of eczema occurs in up to 20% of people with hand eczema and is more common during the spring and summer months and in warmer climates.

Eczema occurs in people of all races and can affect people of any age, although the condition is most common in infants, and about 85% of people have an onset prior to five years of age. Typically, eczema will permanently resolve by age three in only about one-half of affected infants. In others, the condition tends to recur throughout life. People with eczema often have a family history of the condition or a family history of other allergic conditions, such as asthma and/or hay fever. Approximately 20% of children and approximately 1%-5% of adults are believed to have eczema. This means that more than approximately 15 million people in the United States alone express symptoms of the disease and over 32 million people have eczema. Present statistics suggest that eczema sufferers have tripled in the past 30 years and one in five children suffer. It is generally accepted in medical opinion, that there is currently no known cure for eczema, and symptoms can endure a sufferer's lifetime.

While eczema is not known to be contagious, it is believed to be at least partially inherited. As such, it is not uncommon to find members of the same family affected.

Skin diseases such as eczema can be painful and embarrassing. Eczema symptoms generally include incredible itchiness and sufferers typically have poor sleep and liken it to “sleeping on an ants' nest.” In numerous instances, children and infants suffering from congenital eczema have been reported with significant delayed development in speech and growth.

A normal skin barrier is thick and in the stratum corneum the corneodesmosomes are intact and the outermost layers of dead skin cells flake off in a barely detectable manner, as the outermost binders snap and release the unwanted cells. However, in atopic eczema or dermatitis, and other dry skin disorders, the skin barrier is typically thinner than normal so the skin's protective and regulatory capacities are compromised. As the skin barrier breaks down, cracks appear in the skin barrier which allows allergens and infectious agents, such as dust mites and bacteria to enter the skin, thereby causing a worsening of the skin disorder.

Present treatments for eczema include the application of moisturizers between four to six times daily. Healthcare professionals prescribe topical hydrocortisone however this treatment is usually discontinued after seven days, and it is considered a risk for use on children under two years or on irritated or broken skin. Eczema is irritated and broken skin, and sufferers are often under two years. In any case it is known that topical steroids do not cure eczema and often they are applied for years which can result in permanent thinning of the skin, stretch marks, cataracts (if used near eyes) and Cushing's syndrome.

Topical immunosuppressants (topical calcineurin inhibitors) are sometimes prescribed to reduce eczema symptoms. Such products however do not provide a cure, and medical practitioners are hesitant to prescribe immunosuppressants due to documented side-effects. In particular, in 2006, the U.S. Food and Drug Administration warns on product packaging that “a small number of malignancies (skin cancer and lymphoma) have been reported in patients using topical calcineurin inhibitors.”

Typically, more than half of eczema sufferers present with salicylate sensitivity, which worsens after ingesting salicylate-containing foods (found in many healthy foods, fruits and vegetables, sauces and juices). Salicylate- and multiple chemical-sensitivities reduce a person's quality of life and long term adherence to low salicylate diets can be restrictive and can lead to nutritional deficiencies. Eczema sufferers are also known to have elevated histamine levels in the blood combined with a reduced capacity to detoxify these histamines.

Accordingly, the present compositions and methods address these shortcoming, and provide unexpected, rapid, long lasting relief of eczema symptoms.

The term “diagnosis” refers to a relative probability a subject has a given metabolic disorder. Symptoms and diagnostic criteria are summarized herein. Similarly, the term “prognosis” refers to a relative probability that a certain future outcome may occur in the subject. For example, in the context of the present invention, prognosis can refer to the likelihood that an individual will develop eczema. Prognosis can also refer to the likely severity of the disease (e.g., severity of symptoms, rate of functional decline, etc.). The terms are not intended to be absolute, as will be appreciated by any one of skill in the field of medical diagnostics.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the bacteriophages, probiotic bacteria, and/or compounds of the invention. One of skill in the art will recognize that other pharmaceutical excipients are useful in the present invention.

Additional component that may be added to a therapeutic composition of the present disclosure comprising colloidal oatmeal and a bacteriophage include but are not limited to enzymes, ceramides, hyaluronic acid, glycerin, petrolatum, and niacinamide.

As used herein, “colloidal oatmeal” refers to finely powdered, ground oat grain (e.g., Avena sativa) that is heat treated, e.g., boiled, to extract colloidal materials. Currently, the use of colloidal oatmeal as a skin protectant is regulated by the U.S. Food and Drug Administration (FDA) according to the Over-The-Counter Final Monograph for Skin Protectant Drug Products issued in June 2003. Its preparation is also standardized by the United States Pharmacopeia, and is well known in the art. Colloidal oatmeal includes a diverse array of functional molecules, such as starches and beta-glucan, which contribute to the protective and water-holding functions, different types of phenols, which confer antioxidant and anti-inflammatory functions, and saponins, which contribute a cleaning function. Colloidal oatmeal also has an ability to act as a pH buffer, and can assist in modulating skin pH to provide symptom relief and potentially optimize the stability and activity of S. aureus phage. Thus, its many functional properties make colloidal oatmeal a cleanser, moisturizer, buffer, as well as a soothing and protective anti-inflammatory agent. Available exemplary sources of colloidal sources (brands) include, without limitation: Aquaphor, Cetaphil, Cerave, Exederm, Eczema Honey.

“Patient” or “subject in need thereof” refers to a living member of the animal kingdom suffering from or who may suffer from the indicated disorder. In embodiments, the subject is a member of a species that includes individuals who naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human. Thus the methods are applicable to both human therapy and veterinary applications.

The terms “subject,” “patient,” “individual,” etc. are not intended to be limiting and can be generally interchanged. That is, an individual described as a “patient” does not necessarily have a given disease, but may be merely seeking medical advice.

As used herein the abbreviation “sp.” for species means at least one species (e.g., 1, 2, 3, 4, 5, or more species) of the indicated genus. The abbreviation “spp.” for species means 2 or more species (e.g., 2, 3, 4, 5, 6, 7, 8, 9, 10 or more) of the indicated genus. In embodiments, methods and compositions provided herein comprise a single species within an indicated genus or indicated genera, or 2 or more (e.g., a plurality that includes more than 2) species within an indicated genus or indicated genera. In embodiments, 1, 2, 3, 4, 5, or more or all or the indicated species is or are isolated. In embodiments, the indicated species are administered together. In embodiments, each of the indicated species is present in a single composition that includes each of the species. In embodiments, each of the species is administered concurrently, e.g., within about 1, 2, 3, 4, 5, 6, 7, 8, 9, 10, 30, or 60, 1-5, 1-10, 1-30, 1-60, or 5-15 seconds or minutes of each other.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. Treatment can refer to any delay in onset, amelioration of symptoms, improvement in patient skin appearance, etc. The effect of treatment can be compared to an individual or pool of individuals not receiving the treatment, or to the same patient prior to treatment or at a different time during treatment. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques. In embodiments, treatment is effective to reduce at least one symptom of eczema. In embodiments, treatment is effective to reduce the level of one or more symptoms such as, but not limited to itching, burning, stinging skin, redness or rash on skin, flaking skin, trouble sleeping due to skin discomfort. rough bumpy skin, skin discoloration, oozing and crusting. Thus, a treated subject will exhibit a greater alleviation of symptoms in the same amount of time as a corresponding control subject, and/or will exhibit reduced symptoms more quickly as compared to a corresponding control subject (e.g., a subject who is afflicted with eczema but who has received treatment comprising bacteriophage alone, or comprising colloidal oatmeal alone, or a placebo, or no treatment).

In embodiments, compositions of the present disclosure are administered to a subject suffering from eczema in a “therapeutically effective dose.” Amounts effective for this use may depend upon the severity of the disease and the general state of the patient's health. Single or multiple administrations of the compositions may be administered depending on the dosage and frequency as required and tolerated by the patient, and/or as determined by a physician or other medical professional.

The terms “effective amount,” “effective dose,” “therapeutically effective amount,” etc. refer to the amount of an agent that is sufficient to ameliorate a disorder, as described herein. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

### Compositions and Combination Therapy Comprising Bacteriophage

In an aspect, provided herein is a composition comprising, consisting essentially of, or consisting of at least one S. aureus bacteriophage, colloidal oatmeal, and optionally, one or more pharmaceutically acceptable carriers, and/or optionally, one or more additional active agents, ingredients, or components. The S. aureus bacteriophage is not bound to another molecule or a solid support, such as a bead, and is free in the composition.

In an aspect, provided herein is a composition that comprises at least one S. aureus bacteriophage, and colloidal oatmeal, and optionally, one or more additional active agents.

In an aspect, provided herein is a composition that includes active agents consisting of at least one S. aureus bacteriophage and colloidal oatmeal.

In an aspect, provided herein is a composition that comprises at least one S. aureus bacteriophage, colloidal oatmeal, and one or more additional active agents, wherein the one or more additional active agents comprise a ceramide, hyaluronic acid, glycerin, petrolatum, niacinamide, an enzyme, probiotic bacteria, aloe, mineral oil, humectants, lanolin, antihistamines, alpha-hydroxy acids (e.g. glycolic acid), and optionally a pharmaceutically acceptable carrier or excipient.

In an aspect, provided herein is a composition that comprises at least one S. aureus bacteriophage, colloidal oatmeal, and wherein the composition does not comprise a probiotic bacterium.

In embodiments, the at least one additional active agent includes a ceramide. In embodiments, the ceremide is present at a concentration of 2.5% to 10% (weight/volume). In embodiments, the ceremide is present at a concentration of less than 2.5% but greater than about 0.1%, 0.5%, 1%, 1.5%, or 2% (weight/volume). In embodiments, the ceremide is present at a concentration of 2.5% to 10%, e.g., about 2.5%, 3%, 3.5%, 4%, 4.5%, 5%, 5.5%, 6%, 6.5%, 7%, 7.5%, 8%, 8.5%, 9%, 9.5%, or 10% (weight/volume). In embodiments, the ceremide is present at a concentration of less than 2.5% but greater than about 0.1%, 0.5%, 1%, 1.5%, or 2% (weight/volume).

In embodiments, the at least one additional active agent includes hyaluronic acid. In embodiments, the hyaluronic acid is present at a concentration of 0.5% to 2% (weight/volume). In embodiments, the hyaluronic acid is present at a concentration of less than 0.5% but greater than about 0.1% (weight/volume). In embodiments, the hyaluronic acid is present at a concentration of 0.5% to 2%, e.g., about 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% (weight/volume). In embodiments, the hyaluronic acid is present at a concentration of less than 0.5% but greater than about 0.1% (weight/volume).

In embodiments, the at least one additional active agent includes petrolatum. In embodiments, the petrolatum is present at a concentration of 3% to 10% (weight/volume). In embodiments, the petrolatum is present at a concentration of less than 3% but greater than about 0.1%, 0.5%, 1%, 1.5%, 2%, or 2.5% (weight/volume). In embodiments, the petrolatum is present at a concentration of 3% to 10%, e.g., about 3%, 3.5%, 4%, 4.5%, 5%, 5.5%, 6%, 6.5%, 7%, 7.5%, 8%, 8.5%, 9%, 9.5%, or 10% (weight/volume). In embodiments, the petrolatum is present at a concentration of less than 3% but greater than about 0.1%, 0.5%, 1%, 1.5%, 2%, or 2.5% (weight/volume).

In embodiments, the at least one additional active agent includes glycerin. In embodiments, the glycerin is present at a concentration of 2%, or 3% to 8% (weight/volume), (e.g., about 2.5%, 3%, 3.5%, 4%, 4.5%, 5%, 5.5%, 6%, 6.5%, 7%, 7.5%, or 8%) (weight/volume).

In embodiments, the at least one additional active agent includes niacinamide. In embodiments, the niacinamide is present at a concentration of 2% to 8% (weight/volume). (e.g., about 2%, 2.5%, 3%, 3.5%, 4%, 4.5%, 5%, 5.5%, 6%, 6.5%, 7%, 7.5%, or 8%) (weight/volume).

In embodiments, the S. aureus bacteriophage is present in an amount of about 1×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ plaque forming units (pfu). In embodiments, the S. aureus bacteriophage is present in an amount of about 1×10⁶ to 1×10¹¹ pfu. In embodiments, the S. aureus bacteriophage is present in an amount of about 1×10⁶ to 1×10⁸, about 1×10⁸ to 1×10⁹, about 1×10⁹ to 1×10¹⁰, about 1×10⁹ to 1×10¹¹ or about 1×10¹⁰ to 1×10¹¹ pfu. In some embodiments, the amount of S. aureus bacteriophage is provided or described as a concentration (e.g., pfu per mL (pfu/mL)).

In embodiments, a probiotic bacterium is present in the composition. In some embodiments, the probiotic bacterium is present in an amount of about 1×10⁵, 1×10⁶, 2×10⁶, 3×10⁶, 4×10⁶, 5×10⁶, 6×10⁶, 7×10⁶, 8× 10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ colony forming units (cfu). In embodiments, the probiotic bacterium is present in an amount of about 1×10⁶ to 1×10¹¹ cfu. In embodiments, the probiotic bacterium is present in an amount of about 1×10⁶ to 1×10⁸, about 1×10⁸ to 1×10⁹, about 1×10⁹ to 1×10¹⁰, about 1×10⁹ to 1×10¹¹ or about 1×10¹⁰ to 1×10¹¹ cfu. In some embodiments, the amount of probiotic bacterium is provided or described as a concentration (e.g., cfu per mL (cfu/mL))

By way of example, but not by way of limitation, a probiotic bacterium may be one or more of the following: Staphylococcus epidermidis, Staphylococcus aureus 502A, Staphylococcus hominis, and Roseomonas mucosa.

In an aspect, provided herein is a composition that includes an S. aureus bacteriophage, colloidal oatmeal, and an enzyme. In some embodiments, the enzyme comprises an endolysin, an SH3 domain-containing protein, a CHAP domain-containing protein, a lysin, a tail lysin, and/or an anti-inflammatory cytokine. Enzymes such as endolysin can be contributed by the phage itself, and/or can be exogenously added. Exemplary, non-limiting enzymes are well-known in the art, for example, as shown in GenBank AHB79986.1 (putative endolysin of Staphylococcus phage K); NCBI Reference Sequence: YP_009780264.1 (SH3 domain-containing protein of Staphylococcus phage ISP; NCBI Reference Sequence: YP_009780266.1 (CHAP domain-containing protein of Staphylococcus page ISP); NCBI Reference Sequence: YP_008873573.1 (putative endolysin of Staphylococcus phage Sb1); NCBI Reference Sequence: YP_007002194.1 (putative lysin of Staphylococcus phage G15); NCBI Reference Sequence: UP_009275797.1 (tail lysin of Staphylococcus phage Stau2); NCBI Reference Sequence: YP_009275798.1 (tail lysin of Staphylococcus phage Stau2); NCBI Reference Sequence: YP_009275799.1 (putative tail lysin of Staphylococcus phage Stau2); NCBI Reference Sequence: YP_009275801.1. Each of these publicly available NCBI and GenBank sequence entries is incorporated herein by reference in its entirety.

In an aspect, provided herein is a combination comprising, consisting essentially of, or consisting of at least one S. aureus bacteriophage, at least one additional active agent comprising an enzyme (e.g., as described above), wherein each of the at least one S. aureus bacteriophage and the at least one additional active agent comprising an enzyme is in a composition that further includes a pharmaceutically acceptable carrier.

In an aspect, provided herein is a combination that includes a S. aureus bacteriophage, colloidal oatmeal, and an enzyme (e.g., as described above).

In embodiments, the composition or combination includes an S. aureus biofilm degrading enzyme. In embodiments, the enzyme is a glycosidase, a protease, a DNAse, or a restriction endonuclease. In embodiments, the enzyme is a glycosidase. In embodiments, the glycosidase is a glycoside hydrolase. In embodiments, the enzyme catalyzes the hydrolysis of linear polymers of N-acetyl-D-glucosamines. In embodiments, the enzyme is a β-hexosaminidase. In embodiments, the enzyme hydrolyzes β-1,6-glycosidic linkages of acetylglucosamine polymers. In embodiments, the enzyme is a DNAse I, a restriction endonuclease, papain, bromelain, Trypsin, Proteinase K, Subtilisin, serratiopeptidase, dispersin, alginate lyase, amylase, or cellulase. In embodiments, the enzyme is Dispersin B. In embodiments, the enzyme is a protease, and the protease is proteinase K or subtilisin.

Additionally or alternatively, in embodiments, the composition or combination includes a bacterial cell wall degrading enzyme. In some embodiments, the cell wall degrading enzymes comprises a bacterial endolysin. In some embodiments, the endolysin is specific for Staphylococcus and by way of example but not by way of limitation, comprises one or more of GenBank AHB79986.1 (putative endolysin of Staphylococcus phage K); NCBI Reference Sequence: YP_009780264.1 (SH3 domain-containing protein of Staphylococcus phage ISP; NCBI Reference Sequence: YP_009780266.1 (CHAP domain-containing protein of Staphylococcus page ISP); NCBI Reference Sequence: YP_008873573.1 (putative endolysin of Staphylococcus phage Sb1); NCBI Reference Sequence: YP_007002194.1 (putative lysin of Staphylococcus phage G15); NCBI Reference Sequence: UP_009275797.1 (tail lysin of Staphylococcus phage Stau2); NCBI Reference Sequence: YP_009275798.1 (tail lysin of Staphylococcus phage Stau2); NCBI Reference Sequence: YP_009275799.1 (putative tail lysin of Staphylococcus phage Stau2); NCBI Reference Sequence: YP_009275801.1.

Additionally or alternatively, in embodiments, the composition or combination includes an anti-aging enzyme. In embodiments, the anti-aging enzyme is a superoxide dismutase or a peroxidase.

In embodiments, the S. aureus bacteriophage has a linear double stranded DNA genome.

In embodiments, the S. aureus bacteriophage is within the bacteriophage order Caudovirales.

In embodiments, the bacteriophage is a wild-type bacteriophage. In embodiments, the bacteriophage has a genome with a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the genomic sequence of a wild-type S. aureus bacteriophage. A non-limiting example of an S. aureus bacteriophage useful for the present comprises SEQ ID NO: 1, SEQ ID NO: 44, SEQ ID NO: 45, and/or SEQ ID NO: 46.

The therapeutic compositions disclosed herein, comprising colloidal oatmeal, bacteriophage, and optionally, one or more additional active agents, e.g., endolysin. By way of example, but not by way of limitation, therapeutic compositions comprising phage at concentration between 10³ and 10¹² pfu/mL with 1% colloidal oatmeal in one or a combination of the following formulations: an aqueous phase, an organic phase, a water-in-oil emulsion, in an oil-in-water emulsion, in biphasic phase-separated form, or in one of the above formulations with the phage or another ingredient encapsulated or occluded in suspension in the medium.

The combination of bacteriophage and colloidal oatmeal exhibits a synergistic effect in treating a subject's dermal dysbiosis (e.g., eczema). That is, the combination exhibits a treatment effect (i.e., relief from and/or reduction of symptoms), that is greater than each component alone, i.e., bacteriophage alone, or colloidal oatmeal alone, as demonstrated in the Examples.

By way of example, but not by way of limitation, the combination of colloidal oatmeal and bacteriophage allows for increased bacteria killing. In addition, the combination of colloidal oatmeal and bacteriophage will provide at least one or more of the following unexpected effects: increased penetration of phage into the subject's skin and increased persistence of the phage on the subject's skin, thereby increasing contact time of the phage with the target bacteria. The increased phage contact time will allow for increased destruction of the target bacterial and faster treatment/recovery from eczema than provided by prior art compositions and methods, or provided by treatment with phage alone, or provided by treatment with colloidal oatmeal alone. That is, the known benefits of colloidal oatmeal (such as soothing, moisturizing, providing a barrier against further infection, an anti-inflammatory properties), will contribute to the alleviation of symptoms, while allowing the phage a greater opportunity to lyse target cells and treat the dysbiosis, not simply relive symptoms.

By way of example, but not by way of limitation, it is anticipated that at least one symptom of a treated subject's eczema will exhibit a statistically relevant reduction, or reduction in a statistically relevant faster time, as compared to the eczema symptoms of a comparable control subject (e.g., a subject treated with placebo, or treated with phage alone, or colloidal oatmeal alone). By way of example, a subject treated according to the compositions and methods disclosed herein will show at least about 5%, 10%, 15%, 20%, 25%, 30%, 35%, 40%, 45%, 50% or greater improvement in symptoms as compared to a comparable control subject, in the same amount of time. Additionally or alternatively, in some embodiments, a subject treated according to the compositions and methods disclosed herein will show improvement in at least one eczema symptom in at least about 5%, 10%, 15%, 20%, 25%, 30%, 35%, 40%, 45%, 50% less time than a comparable control subject.

In embodiments, a subject diagnosed with, suffering from, or suspected of having dermal dysbiosis, such as eczema, is treated with a composition of the present technology, i.e., a composition comprising a bacteriophage and colloidal oatmeal. The composition is applied topically to the skin of the subject. In some embodiments, the composition is applied to at least the affected area of the subject's skin (e.g., the area exhibiting eczema), and in some embodiment, the composition is applied to the skin surrounding the affected area.

In some embodiments, the composition is administered once per day, twice per day, three, four, five, or six times per day, or as needed. In some embodiments, the composition is applied over a period of one week, two weeks, three weeks, one month, two months, three, four, five, six, seven, eight, nine, ten, eleven months, or for a year or more. In some embodiments, the composition is applied, daily, as needed, e.g., until symptoms of the dermal dysbiosis (e.g., eczema) are reduced or absent. It is anticipated that the subject treated with the compositions of the present technology will exhibit reduced symptoms of dermal dysbiosis (e.g., eczema) sooner than a comparable subject that is not treated with the disclosed compositions.

Miscellaneous. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following examples are illustrative and should not be interpreted to limit the scope of the claimed subject matter.

### Example 1: Higher Stability: Phage is Preserved for Longer in the Presence of Colloidal Oatmeal (CO)

S. aureus phage MESA-11 (SEQID 45) was prepared in either PBS or PBS+1% colloidal oatmeal (CO). Each formulation was incubated in triplicate at 30 C for 48 hours, the phage concentrations were determined by titer assay and compared to the initial concentration at t=0. It was found that despite starting from the same concentration, 1% CO conferred 2.64×(264%) higher stability than phage in PBS alone.

Temperature: In a first experiment, S. aureus bacteriophage formulations with or without CO are tested for stability by incubation at room temperature and elevated temperatures. In one or more of these conditions, it is anticipated that the presence of colloidal oatmeal will prolong the stability of the S. aureus bacteriophage.

pH: In a second experiment, S. aureus bacteriophage formulations with or without CO are incubated in buffers at different pHs. It is anticipated that the S. aureus bacteriophage will show better survival at alkaline pHs (as is found on eczema skin) due to the acid-buffering capacity of colloidal oatmeal.

### Example 2: Protects the Phage: CO Prolongs the Presence or Activity of Phage on the Skin

Several topical formulations (A-F) will be prepared and evaluated as follows:
A: Phage with 1% CO in aqueous phase formulation.
B: Phage with 1% CO in organic phase formulation.
C: Phage with 1% CO in oil-in-water emulsion formulation.
D: Phage with 1% CO in water-in-oil emulsion formulation.
E: Phage with 1% CO in discontinuous aqueous and organic phase formulation.
F: Phage with 1% CO in a formulation where the phage is encapsulated or occluded and in suspension.

S. aureus bacteriophage formulations A-F, prepared with or without CO are applied on the skin of human subjects. After a certain period of time, the area of application is swabbed and the surviving phage is quantified using a phage titer assay. It is anticipated that the colloidal oatmeal will positively affect the survival of the S. aureus bacteriophage on the skin.

### Example 3: CO Enhances Phage Penetration into the Skin

S. aureus bacteriophage formulations A-F, prepared with or without CO are applied on the skin of human subjects. Upon complete absorption, the area of application is subjected to tape stripping to isolate layers of stratum corneum. Each tape strip is titered to quantify the S. aureus bacteriophage within successive layers of the stratum corneum. It is anticipated that the formulations containing colloidal oatmeal will show deeper penetration into the skin, evidenced by the persistence of S. aureus bacteriophages in deeper layers of the stratum corneum in formulations containing colloidal oatmeal.

### Example 4: CO Enhances the Killing of Bacteria

S. aureus phage (MESA-11 SEQID45) was added to a logarithmic phase culture S. aureus strain UAMS-01 in BHI broth, in the presence or absence of 1% CO. The cultures were incubated aerobically at 37 C in triplicate. After 24 hours of incubation, the survival of S. aureus bacteria was measured by plating serial dilutions on a BHI agar plate. After overnight incubation at 37 C, the S. aureus survival was calculated by counting colonies in the relevant dilution and multiplying by the dilution factor and the inverse fraction of 1 mL plated. It was found that on average in the presence of 1% CO, S. aureus survival was 59% of the comparative survival in its absence (0.73×10¹⁰ pfu/mL vs 1.23×10¹⁰ pfu/mL, respectively).

S. aureus bacteriophage formulations A-F, prepared with or without CO were incubated with S. aureus host bacteria. The formulations will be assessed and compared for their efficiency in killing S. aureus bacteria, by determining the growth kinetics, phage expansion, and survival of host bacteria. It is anticipated that the presence of colloidal oatmeal will enhance the killing of bacteria by S. aureus bacteriophage.

### Example 5: CO Calms the Skin Upon Phage Application

S. aureus bacteriophage formulations A-F, prepared with or without CO are applied on irritated or inflamed skin. After repeated applications, the affected area is evaluated to quantify the inflammation. It is anticipated that the presence of colloidal oatmeal will reduce inflammation more than S. aureus bacteriophage alone.

It is anticipated that the combination of the S. aureus bacteriophage with colloidal oatmeal will achieve more than an additive effect, i.e., a synergistic effect (the combined effect of the bacteriophage and the colloidal oatmeal is greater than the sum of the effects of the bacteriophage and the colloidal oatmeal when each agent is used separately) in treating skin irritation or inflammation, such as with eczema. The effectiveness of treatment is measured by evaluating the patient's skin for decreased pain, itchiness, bumps, oozing, discoloration, and general discomfort.

### REFERENCES

1. Estrella, Luis A, et al., Characterization of novel Staphylococcus aureus lytic phage and defining their combinatorial virulence using the OmniLog® system, Bacteriophage, 6 (3), e1219440 (13 pages) Aug. 5, 2016; doi: 10.1080/21597081.2016.1219440.
2. Ajuebor, J. et al., Comparison of Staphylococcus Phage K with Close Phage Relatives Commonly Employed in Phage Therapeutics, Antibiotics (Basel) 2018 Apr. 25; 7 (2): 37. doi: 10.3390/antibiotics7020037. PMID: 29693603; PMCID: PMC6022877.
3. Gu, J. et al., Genomic characterization of lytic Staphylococcus aureus phage GH15: providing new clues to intron shift in phages, J Gen Virol. 2013 April; 94 (Pt 4): 906-915. doi: 10.1099/vir.0.049197-0. Epub 2012 Dec. 19. PMID: 23255621.
4. Ganaie, M. Y. et al., Isolation and characterization of two lytic bacteriophages against Staphylococcus aureus from India: newer therapeutic agents against Bovine mastitis, Vet Res Commun. 2018 December; 42 (4): 289-295. doi: 10.1007/s11259-018-9736-y. Epub 2018 Sep. 15. PMID: 30219981.
5. Natalia Łubowska, et al., Characterization of the Three New Kayviruses and Their Lytic Activity Against Multidrug-Resistant Staphylococcus aureus, Microorganisms, 18; 7 (10); 471, October 2019.
6. Sue-Er Hsieh, et al., Wide host range and strong lytic activity of Staphylococcus aureus lytic phage Stau2, Hsieh, Sue-Er et al. “Wide host range and strong lytic activity of Staphylococcus aureus lytic phage Stau2.” Applied and environmental microbiology vol. 77, 3 (2011): 756-61. doi: 10.1128/AEM.01848-10.
7. Kim, M S, et al., Complete genome of Staphylococcus aureus phage SA11, J Virol. 2012 September; 86 (18): 10232. doi: 10.1128/JVI.01574-12. PMID: 22923794; PMCID: PMC3446589.
8. Gill, JJ. Revised Genome Sequence of Staphylococcus aureus Bacteriophage K, Genome Announc. 2014; 2 (1): e01173-13. Published 2014 Jan. 23. doi: 10.1128/genomeA.01173-13.
9. Gu, J, et al., Complete genome sequence of Staphylococcus aureus bacteriophage GH15, J Virol. 2012; 86 (16): 8914-8915. doi: 10.1128/JVI.01313-12.
10. Sassi, M. et al., Genome Sequence of the Clinical Isolate Staphylococcus aureus subsp. aureus Strain UAMS-1, Genome Announc. 2015; 3 (1): e01584-14. Published 2015 Feb. 12. doi: 10.1128/genomeA.01584-14.
11. Shimamori Y., et al., Isolation and Characterization of Novel Phage SaGUI that Infects Staphylococcus aureus Clinical Isolates from Patients with Atopic Dermatitis, Curr Microbiol 78, 1267-1276 (2021).
12. Kwan, T. et al., The Complete Genomes and Proteomes of 27 Staphylococcus aureus bacteriophages, PNAS 102 (4), 5174-5179 Apr. 5, 2005.
13. Göller, P. C., et al., Multi-species host range of staphylococcal phages isolated from wastewater. Nat Commun 12, 6965 (2021)

1. A composition formulated for topical administration comprising:
a) a Staphylococcus aureus (S. aureus) bacteriophage comprising a nucleic acid having at least 90% sequence identity to SEQ ID NO: 45, and
b) colloidal oatmeal.

2. The composition of claim 1, further comprising one or more additional active agents.

3. The composition of claim 2, wherein the one or more additional active agents comprises a ceramide, hyaluronic acid, glycerin, petrolatum, niacinamide, an enzyme, and a probiotic bacteria.

4. The composition of claim 3, wherein the enzyme comprises a bacterial cell wall degrading enzyme.

5. The composition of claim 3, wherein the enzyme comprises an endolysin.

6. The composition of claim 3, wherein the enzyme comprises an anti-aging enzyme.

7. The composition of claim 1 consisting of the S. aureus bacteriophage and the colloidal oatmeal.

8. The composition of claim 1, further comprising one or more additional S. aureus bacteriophage.

9. A method of treating eczema in a subject in need thereof, the method comprising:
topically administering the composition of claim 1 to a region of the subject's skin comprising the eczema.

10. The method of claim 9, wherein administration comprises once daily, twice daily, three times daily or four times daily administration.

11. The method of claim 9, wherein the composition is administered for one week, two weeks, three weeks or more.

12. The method of claim 9, wherein treating comprises reducing one or more of the following eczema symptoms: itchy skin, dry skin, redness, and swelling of the region of the subject's skin administered the composition.

13. The method of claim 12, wherein reducing the one or more eczema symptoms occurs more quickly than in a control subject.

14. The method of claim 13, wherein the treated subject exhibits improvement 5%, 10%, 15%, 20%, 25%, 30%, 35%, 40%, 45%, or 50% faster than the control subject.

15. The method of claim 11, wherein the treated subject exhibits about 5%, 10%, 15%, 20%, 25%, 30%, 35%, 40%, 45%, 50% or greater improvement in at least one symptom as compared to a comparable control subject, in the same amount of time.

16. The method of claim 9, wherein the eczema is selected from the group consisting of contact eczema, allergic contact eczema, seborrheic eczema, nummular eczema, neurodermatitis, stasis dermatitis, dyshidrotic eczema.

17. The method of claim 9, wherein the eczema is atopic eczema.

18. The method of claim 9, wherein the subject is human.


SEQUENCE LISTING (46 sequences)

SEQ ID NO: 1 - DNA, 17465 bp, Staphylococcus phage sp.
  source 1..17465: mol_type = genomic DNA; note = MESA-01
agggaaattttttgtgtaaatttacactaccccaccgtttaaaataaacgattaactaattcataacttattaatcaacttataaattcttttataatcttatataatctgtgctactactaataacatctattcgaaacattcaaatattctttaaaccaaaaaatattcaattgtaaattcaaataataaaaatataattatgttttaaaacaacatcacaactttatatacaactttaaataatttatgatataataagttataaaatattttggaggtgtatattaaatgagtgagtttgaagaaattgttaaatctgatgaagaaactgaagaatcaactgaagaatcaactgaagaatcaactgaagaatcaactgaagaatctactgaagataaaacagtagaaacaattgaagaagaaaatgaaaacaaattagaaccaactacaactgatgaagatagtgcaaaattagaccctgttgttttagaacaacgtattgcttcattagaacaacgatttactaattttgaaacatcacaaatgcaacaaacacaacaagtagaatcaactacaccagatgtaactaatgctgataaagaagacaaagactattcagatgaagaattagtagataagttagatttagactaggaggaaaataatatgtatgaaggtaataacatgcgttctatgatgggaacatcatatgaagattcacgtttaaacaaacgtacagaattaaatgaaaatatgtcaattgacgttaataaaagcgaagatagttatggggttcaaattcattcactatctaagcaaacatttactggagacgtagaggaggaataataaattatggctacaacaaaaaatgaaactgccctattagttgctaaatcagcaaaagcagcattacaagattttaaccatgattattcaaaatcttggacttttggtgataagtgggataatacaaacacaatgtttgaaacatttgttaataaatatttattccctaaaattaatgaaacattattaattgatattgcattaggtaaccgttttaattggttagcaaaagagcaagactttatcggacaatattcagaagaatatgtaattatggatactgttccaattaatatggacttatcaaaaaacgaagaattaatgttaaaacgtaattatccacgtatggctactaagctatatggtagtggtattgtaaaaaaacaaaaattcacattaaacaacaacgacgttcgttttaatttccaaacattagcagacgctacaaattatgcgttaggtgtttataaaaagaaaatttctgatattaatgtattagaagaaaaagaaatgagagcaatgttagttgattactcgttaaatcaattatcagattcaaacatacgcaaaacaacatcaaaagaagatttagcaagtaaagtatttgaagcgattcttaacttacaaaacaacagtgcaaaatacaatgaggtacatcgtgcttctggcggcgctattggtcaatacacaactgtgtctaagttaaaagacattgttattttaacaacagattcattaaaatcttatttattagatacaaaaattgcgaacactttccaaatcgctggtattgacttcacagaccatgttattagctttgatgatttaggtggcgtgtttaaagtaacgaaagaatttaaattacaaaatcaagatacaattgatttcttacgtgcttacggtgattatcaagcacaaatcggtgatactattcctttagatgctgtatttacttatgacgtttcaaaacttaaagagtttacaggtaacgttgaagagattaaaccaaaatcagatttatatgcgtttattttagatattaattcaattaaatataaacgctatacaaaaggtatgttaaaacaaccattctataatggtgagtatgatgaggttacacattggatacattactattcatttaaagctgtaagcccattctttaacaaaattttaattactgaccaagatgttacacctaaaccagatactgtttcagaataggaggtaaattatgacaaatagcaaaagaggtttagatgttgaactgtcaaaacaaattaataaaagagtagttgaacatcgtaaccgttttaaacgtcttatgtttaatcgttatttggaatttttacccctactaataaactataccaatcgtgatacggttggtatagattttattcagttagaatccgcattaagacaaaacattaatgttgtggtaggagaagcgagaaataaacaaattatgattcttggttatgttaacaatacttatttcaaccaagcacctaatttttcttcaaactttaatttccaattccaaaaaagattaacaaaagaagatatatattttattgttcctgattatttaatacctgatgaatgtctacaaattcataagcaatacgataattgcatgagtgggaattttgttgttatgcaaaataaaccaatacaatataacagtgatattgaaatcatagaacattacacagatgaattagctgaggttgttttatctcgtttttcattgattatgcaagcaaaattcagcaagatatttaaatcagatattaacgatgaatcaattaatcaacttgtatcagaaatttataacggtgcaccatttgttaagatgtcaccaatgttcaatgcagatgatgatattattgatttaacaagtaattcagttattccagcattaacagaaatgaagagagaatatcagaataaaattagtgaattaagtaactatttaggtattaattcactagctgttgataaagaaagcggtgtttctgatgaagaagcaaaaagtaatcgtggttttactacgtcaaacagtaatatctatttaaaaggacgtgaaccgattacatttttatcaaaacgttatggtttagatattaaaccgtattatgatgatgaaactacgtctaaaatttctatggttgatacactgtttaaagatgaaagtagtggtgaaaatggctagatataccatgacattatatgattttataaagtcagaactgattaaaaaaggttttaatgaatttgtaaataacaataaattaaccttttatgatgatgaatttcaatttatgcaaaaaatgcttaaatttgataaagatgtacaagcaattgtaaatgaaaaagtatttaaaggtttttcacttaaaaatgagttatcagatttactttttaaaaaatcatttacgattcactttttagatagagaaattaacagacaaaccgttgaagcgtttggcatgcaagtgattacagtgtgcataacacatgaggattatttaaatgttgtttattcatctagtgaagttgaaaagtatctacaagcacaagggtttactgaacacaatgaagatacaacaaacaatactgatgaaacatcgaatcaaaatgctacatcattagacaattcaacaggtatgactgcaaacagaaatgcttttgtgacattacctcaaagtgaggttaatatcgatgttgataatacaacattacaatttgctgataacaataccattgataatggtaaatcagtgaataaatcaagtagtgaaagtaatcaaaacgcaaaacgtaatcaaaatcaaaaaggaaatgctaaaggtacacaattcacaaaacaatacttaatcgaaaatatcgataaagcatatgatttaagaaaaaaaatattaaacgaatttgataaaagatgttttttacaaatttggtaaggtggttaaataatggcatataatgaaaatgattttaaatattttgaggatattcgtccattcatagatgagatatataaaacaagagaaacatatacaccgttctatgatgatagagctgattataatactaattcaaaatcgtattatgattatgtttcaaaactatcacgactgattgaagtattagcacgtcgtatttgggaatatgacggtgaattaaaaaaacgtttcgaaaattgggacgatttaatgaaacaatttccagatgacgctaaagaattatttagaggatggttaaacgacggcacgattgataaaatcattcatgatgagtttacaaaatattcagctggtctaacttcagcttttgctgtatttaaaattgctgaaatgaaacaaatgaatgattttaaagcagaagttaaagacttaattaaagatattgaccgtttcgttaatgggtttgaattaaatgaacttgaaccaaagtttgttatgggattcggtggaatacgtaacgctgttaaccaatctgttaatattgataaagaaacaaatcatatttatacaacacaatcagactcacaatctccagagggcttttggataaataaattaacccctagtggtgatttactttcaagtatgcgtatcgtacaaggtgggcacggcacaacagtaggtttagaaagacaatcaaacggtgaaatgaaaatatggttacatcatgacggtgtagctaaactattacaagtagcttataaagataattacgttttagatttagaagaggcaaaaggtttaacagattatacaccacaatcacttttaaacaaacatacatttacaccattaattgatgaagcgaatgacaaactcattttacgtttcggagatgggacaatacaggtacgttcaagggtagatgtaaaaaatcatattgataacgtagaaaaagaaatgacaattgataattcagaaaataacgatacacgctggatgcaagggatagctgttgatggggatgatttatactggttaagtggtaatagttctgttaattcacatgttcagattggaaagtattcattgaaaacaggggaaaaaatatacgattatccgtttaaattatcatatcaagatggtattaacttcccacgtgataatttcaaagagccagagggtatttgtatttatattaatccaaaaacaaaacgtaaatcattattacttgcaatgacaaatggcggcggtggtaaaagattccataatttatatggattcttccaaactggagaatatgaacattttagtgccttacgtgcaagaggtgcacaaaactataaattaacaaaagatgatggacgcgctttatcaataccagactatatagatgatttaaacaacttaacacaagcaggattttattatatcgatggtggaacagcaggaaaacttaaaaacatgcctacaaacggtagtaagaaaattattgacgcaggttgctttatcaatgtataccctacaacacaaacattaggaacagtgcaagaaataacacgtttctcaacaggtcgtaaaatggttaaaattattcgaggtatgacgttagatgtgtttacattaaaatgggattatggtttatggacaacaatcaaaacagacgcaccatatcaagaatatttggaggcaagtcaatacaacaattggattgcatatgtaacaacaccgggtgagtattacattacaggtaatcaaatggaattatttaaagacgcaccagatgatattaaacacgttggtgcatggttgaaagtgtcaagtgggaatgctgttggtgaggttagacaaacattagaagcaaatatagctgaatataaagaattctttagtaatgtcaatgcggaaacaaaacatcgtgaatacgagtgggtagcaaaacataaataggagatgaaacaatgaagtcattacaacaagcaaaacaatggattgatgtcaatactggtagaggcattgactttgatggcgcatatggatttcaatgtatggatttagctgtagcttatatgtattatgttactgacggaaaagtgcgtatgtggggtaatgccaaagacgcaattgataataactttaaaggtttagctaaggtgtatcaaaatacacctagctttaaacctcaattaggtgatattgctgtttatacaaactcacaatatggtcatattcaagttgtgattagtggtaatttagattattatacatgtctagagcaaaactggttaaatggtggttatgacggttgggaaaaagcaacaatcagaacacattattatgacggtgtaacacactttattcgtccaaacttttcaaatagtaatagtaaagtattagaacaaaacattcaacaaacaaataaatggaaacaaaatcaatacggtacattatataaatctgaaaatggtacatttacatgtggatttttaccaatatttgcacgtgttggaagtcctaaattaagtgaaccgaatgggtattggttccaaccaaacggctatacaccatatgatgaggtttgtttatcagatggactagtgtggattggttataattggcaaggtacacgttattatttaccagtgagacaatggaacggtaaaacgggtaatagttatagcattggtttaccatggggggtgttctcataatgggtattttaggttttttctttgagtttagttggaaaagatacaaataagaggtgtaaaatatggctgatagaatcgtaagaagtttaagaggtattgattcagtagagaagttaaacgacaatttagtagaagcaaacgacttaatcacaaccaaagacgataacatatatataagacgtgatgaggattattataagctaacatttaaagatgaattattagaaaaaatcaatacaaacacaaaagcgattgataaaaataaaaatgacatcactacaaataaaaaaaatatatctcaaaacgctacagatattattaacattaaagaagataatattcaacaagataaaaaaattaaaaatttatctgacgttcaaacagaacatgaaaatacattaaacaatcatgatgacgcaattcgtttattagatgatgaaaatacaaaaaacaaattagcaattgaaaagaataaacaagatattatcgctacaaaagatacaatcggacaaaataaacaaagtattgaaaacttagcttcaacggtttcaaacaacacgattgaaacaagtaaaaaaattgaatcaactaaaacagaattacaagaacaaatcaaatcttcaaaaacaagtgtgaatgatacaggttggttaaatattcaacttgaaagtggtatcacagcaagtgattcaagtggtggatattccacaccacaatatcgaattatagatataaataacatcagaactattcaattaagaggtgtattaaaaggtgttaagaaaaatggagatattaaattaggcacgattaatgctaatttaaaagtaacacatcactatacacaatgtgctattgattctaaaatgataaacacaagattatatgtaaactttaataacgaattacattttgttacatcaggttatcaagatagtgaactttcttctggagataaacgttttgttattgatacacaaatcattgaataaaaatgatataatagtcgtataaattattttatacgactattttatttggaggaaaatctatgaggaaattaacaaattttgtatttttttataatacaccttttacagattatcaaaatacaatacattttaatagtaataaagaacgagatgattattttttaaaaggtcgtcattttaagtcattagattattcaaaacaaccgtataacttcattcgtgatagaatggaagttaatgtagatttaagttggcatgacgcacaaggtattaactatatgacgtttttatctgattttgaaaatagacgttattatgcgtttgtgaaccaaattgaatatattaacgatgttacgacaaaaatttattatgtcattgatacagttatgacgttcacacaaggtaatgtgttagagcaactctcaaacgtcaatattgaacgacaacatttatcaaaacgtacgtataactatatgttaccaatgttaagaaacaacgatgatgttttaaaagtaagtaataaaaactatgtttataaccaaatgcaacagtatttgcaaaatgttgtactatttcaatcaagtgctgatttatctaaaaagtttggtactaaaaaagagcctaatttagacacatcaaaaggaacaatatatgacaatatcacatcaccagttaatttgtatgtcatggaatataatgattttattaactttatggataaaatgagcgcatatccgtggattacgcaaaacttccaaaaagtacaaatgttacctaaagactttattaacgaaaaagatttagaggacgttaaaacaagtgaaaaaattactggtttaaaaacattaaaacaaggtggtaaatctaaagagtggagcttaaacgatttatcattaagtttttcaaaacttcaagaaatgatgttgtctaaaaaagatgaattaaaacacatgatacgcaatgaatacatgaccattgaattttatgattggaatggtaatacaatgttacttgacgctggtaaaatttcagaaaaaacaggagtgaaactaagaacaaaatctattattggttatcataatgaagttagagtttatccagtagactataacagcgctgaaaacgatagaccgatacttgctaaaaacaaagacatattaattgatacaggttcatttttaaatacaaatattacatttaatagctttgctcaagttccaattttaattaataacggtatattaggacaatcacaacaagcaaatagacaaaaaaatgcagaaagtcaattaatcacaaatcgaattgataacgtgttaaatggtagtgaccctaaatcaagattttacgacgctgtcagtgttgctagtaatttaagtccaacagctttatttggtaagtttaatgaagaatataatttttataagcaacaacaagctgaatataaagatttagcactacaaccaccgtctgtgacagaatcagatatgggtaatgcattccaaattgcaaataatattaacggtttaacaatgaagattagtgtaccgtctccaaaagaaattacattcttacaaaaatattacatgttatttggttttgaagtgaatgactataacaatttcattgaacctattaatagtatgactgtatgtaattacttaaaatgtaacggaacgtatacattacgtgatattgaccctatgttaatggaacaactcaaagcaatattagaaactggtgtgagattttggcataatgacgggtcaggtaatccaatgttacaaaatccattaaataataaatttagataggggttttaattttgaatgaagtaaaagtaagatttacagatacagaagcatttcatatgtttatttatgcaggagatttaaaattattatatttcttatttgtattaatgtttatcgacattattacaggtatagcaaaagccattaagaataataatttatggtcaaagaaatctatgaaagggtttgctaaaaaattactcattttttgtattattatactagcaaatatcattgaccaaattttacaattaaaaggtgggttactcatgataacgatattctattatatcgcaaatgagggcttatctattgttgaaaattgtgcagaaatggatgtattagtgccagagcaaatcaaagaacgattaaaagtaataaaaaacgagtctgaaaagagtgataacaatgaacgaccaagagaagattgataaatttacccactcgtatattaatgatgacttcggtttaacgatagaccaacttgtaccaaaagttaaaccttacgggcgatttaatgtatggctaggtggtaatgaaagtaaaataagacaagtattaaatgcagtaaagtcgataggtgtttcaccaaccctttttgctgtttatgaaaagaatgaagggtatagtgcaggtttagggtggttaaaccatacatctgcacaaggtgattatttaactgatgctaaatttgtagctagaaaattagtatcacaatcaaaacaagcaggacatccgtcttggtatgatgcaggtaacattgtgcattttgtacctcaagacgtacaaagaaaaggtaatgaagattttgctaagaatatgaaagcaggtacagtcggacgtacctatattccattaacggcggctgctacttgggctgcttattacccactaggtctgaaagcttcatataatagagtacaaaactatggtaatccatttttagacggtgctaataccattttagaatggggcggtaaaattgacgggaaaggtggttcacctagtagtggctcatctgatagtggttctgatagtggaggaaattcattactagccctagcaaaacaagctatgcaagaattattaaaaaaagtgcaagacgcattacaatgggacgtacacagtatcggtcatgataaatattttagtaatgattattttacattagaaaaaacgtttaacaatacgtatcatattaaaatgactatagggttacttgattcactcaaaaagattatagatagtattcatattgacagcggtggtagctcatctaatcctactgatgatgacggagatcataaaccaattagtggtaaatctgttaaacctaacggaaaaagtggtcgtgttattggtggtaactggacatatgaccaattacctgaaaaatataaaaaggcaattggtgtacctttgttcaaaaaagaatatttatataaaccgggtaacatattccctcaaaccggtaatgcaggacaatgtacagaattaacatgggcgtatatgtcacaattacatggtaagcgtcaacctacagacgacggtcaaattactaacgggcaacgtgtatggtacgtttataagaagttaggtgcaaaaacaacccataacccaacagtaggttatggtttttcaagtaaaccaccatacttacaagcaagtatttatggtattggacacacaggtgttgtcgttgctgtatttgacgatggttcattcttagttgcaaactataacgtaccaccttacgttgcaccgtcacgagtgttattatatacactaattaatggtgtaccagaaaatgcaggtgataatattgtattctttagtggtattgcttaacaagttatgctataatgtaactatgctagattactagtaaataaaatacaaaacataatcaattttcgtacacattttttattgttatctcaaacttaaagggtagccgttattttaacggttacccttttcgtatgtcatcatgtatttgttttaattgatttaactttgaacgatattttgcagaacgttctactgggaataaatcatttaatgaaaatgtgccaatatcactttcaatatatagaatatcatcatattcactatgttcaaaattttctctagcgtcttttagcaaaaattctcttttttcatttaattcatctgaaaagacatcatcatatacatctccgcttacaatctcagttttagacgggtaaatcgaaatcgtaccctgctcattatagatacttttattgttgtaaacaacagcaccatcaaaaaattgttcactgacaaatgtttcaaaatcaacgcttgtatcaaaagcgtttttcggtataccagcagaagcgattttaatttttccgttcgtcacataagcatatttcttatgattgagtacaaacatcttatctatctgttcgttttcaatatcccatttacctaaggcaatcgggtcgaataaatcggggtttaacaagggtttcacaacggatttcatatacaaactatcagtatcacaataaatgaaattatcgtctatttcactttcagttaagtattgaaacggaactaataagttatataatgagcgtgatgttacaaaagtagaaaatagtaaattacgttctgtgtttttgtatccgtttatcatattaaataattcgttgttttcatctaaacggaataaattaaagtgtgaacgtaaagcaggaataccatataaaccgtttaaaacgactttagacagcataacctcttcatttgagtacgggtgttcgttaatatcatcagtgattttatagtcataaggtgaagtcatattaatttttgttttgagtttcccttgtgttttaataaaatagttttgaaatataatatcacgagcatgaaagtattcacattcatacatcacaaatgaattgacacgtatttttgtgcaatcaatatctgtaatgtcttgaatcattcttaatgtatttgtgttgatattaacgtaatcattatcattattatagtattttacaatcatttgacgtaatacacgtgattttatttttagtaatatatcatggttaaatgtatgcttatcaatcttatataacgtaaaataattatcatcatctaaaaaagtaggaattaacgttggttctgaatagtgttcgtaaaagtataaccatgttggaattttttcatgatacatcacatacggataacttgaattgatatcaatagaaaaacaaggttcatcaacgattttgttaatatactttgtgttatacatatttaacccaccacgataaaatgatttaatataatcataaaaattcatatcatggaattgataatgcgtatatgaaatagcgatgtcatcgtatttattaagtaattgaaaacgtgtcatttcattatttaaatatgattccataatatttaatgaaaacgttaatttattataatcaaaatttggaaaaatatcactataatgtatatgacacatacctaaaataattacatcgttatgaatatatgtaagttgttctgatgttaattttgtaaagcatttagtagcatagtcataagcttcactgtctgacatatctgtttctttatcaaagattgtatagttaaaatcagttttgagttggttttctgtaagataaccaccgtctaataactttttgccaagtgtagcgattgatgtattggttttcataaagttatcaatgatattgaatttaaatccgtttaaaaacatcgttaaatctaaattaatagaggactttacacgtttttcaagtatgacattttgacttttagataatatttgagcttctttcatttttagggtgtgttcattatcttcagctgattttaaaaacacatttttacgtgtaatattatcaaagtaacgcatcgtatctttaagtaaaaaatgattgtcatacttattacagttatgtgcaatcataataatgtctgtttttgattttgtgattgtatcacgtcgtttcacatacttataaaacgaatcataaaatgcttcaaaactagggaatacctctacatcaatttcataaccattaaaccaaccaatagcaactgaatacgtgacgtttttatatttagttggttttttacgtccattgattttattatatgctaatgtttcaatatcccaatataaaatcattttacgttcatgtttatgatattgcatacactcaagtaatcccataatcttacacacctttattgttcgtattgttgcattaagtagcgttttgtattttctatatagttatcttcatatatcttttcttttcgttcaaactcactcatgtttttcttcatttcatttttaatatgaaattttataattttattcacatctaaatataaatacctatcattatcaacaacataattttttgaatatgcgttatcaaaatgtaaatttgacggattataataataacgctccatattttctttataaaacatatcttctctaagatatgtgacgttttcatcaatatctttgattttcgtacaaaattcatattcttttgtataaggtattacaataatatttgctataaatgtagatacattatacataattttaatgaatttatcttctgttttgatatagaaaaaatcaccgttttgattaatatgtgaccgtatactatcgttagctaaattatattcattaaaatcaaattcccctgttgtcatagcgtcctcatttgaattaaacgcacgtgtattacgtttttcgttgacataatcattacgtctcatctctaaaaaaatgtttttataaagtcgtgacgtgttcattttatgtttttgtaatagattatatatgtttaaattagataaaataggacttgaaaagttaacagcgttaccaagtaaaaatattttaggaaatccgatataatctacattaccatgatttctatcaatggactcataaattgttttgagtttatcccactcatcatataaataatcatcttcaagtgctaaaaactcatcatatataatgataggatagtgttttaaaaagttagaatgatatttcaaatcagttgcactatttaaatctgttatcacacctatttctttatcttggtatacaatcgctaaataatctcttgcgcttctgaaagcaacacgttttgatttaaataatggatttttatcaataatttcctcaataaaatcacggtatgcatcacgtaacgtgtaatgacgtgatagtaatgtaaatttaatatcaagtttcattgctaaataaataaaaaacgatacatagttaaatgatttaccgtctgaacggtttgaaatagaaatatagtaatctaaatcatcattcattaattcattgactaattcaatttgattataattatcaggtatcttttttcttacatgattgacaacattttgataatcccttaccatgtctagcctgttttgttttaccatgattatgctccttatagtaatttataatatcttcaattgatttaaaatgtttcatttttctattaaatgataatataaaaagttatatctgacttttttgtttttgattgttgttttcggtctgttagattcaccaatttcatcaaataattcttcaatacctttgatatatttatacattgatgtattattatctttagcagtttcatcatataaagtatatttatgtgtttttgcatttttgttattactatcatcattacgttcataaatgtttaatatttttaacaattgttcacgtcgtttaccacgtactaaagaaacagcatttacataactcacatgtttttctttaggtaaaaatgtaagctttgaaaaatattcccaagcgtcaatatattctttttgtagtgatgcatcttcaaatttaaaactgacaccactaaagtcattcttttttcttttttcttcatctgcttttcttgcttctcgttcttttttccaacggtctgcttctgacatacgtttacgcattgttatcaacctccatatatagcataaataaccattaaaaagataatatagaatatgattagtgttgtaaatagtacaccaaatgacacacgtatatgcatggtcataagaatgacaagtgtaatcaataatgctaaaagaaaaatgataattatattaagtaggttattcatgttctatcacatttccgtttttatatataactttgttttggtaaataatcattaatccgctttcgagtggacaatcaaaatttactaatacatcgtctattgtaacaccatatgaagtttctcttacaaatgtgttatttacagaatctatataataatactcaagtaaataaaaaacatgttttttaatatcaatatcaatatctaacgtgaataaatcattgttaattgttaagtccgtattatgactttcagaaaaattaacacaaatatcatcatcgctatcaatacctacaaaattataaccactatcagaaataaattgtatttttttatcttgaaaattttttctgttatcgttaatgtagcaaagtaagtcatataaattaaatgattgttttattttcattttaagtctcctctatatattctataacatacgcataattattatgaaatggattgaattcataataagagtataaaactttggcgttatacaaatcttcaaacattaatatttgatgatgaaaatcattttctattgtgtcagtatataataagactgttttatattttttcattatatcctccaataaaaatagagggaatgaaatccctctatgaaattttaatattaaaatgatacttgactaaagttaatagaatatcctttttgaccttttttgttttcgtattcataaattgagaacacaacttcacctttattaataatgtctacaacatcttcatcttgacgcatgtctttaattaattcagttaagtgattcggtaagttaacattataatcatctgttacaataacaccttgttcaccgaattttgattctttgtttgtaaataatgctctaacgatgtattcttgtttgattccgaatttttcaactaattccgataatttgataaattctctttctttttcctcaaattcaaatctcgctaatgtgttttggtgtctagataaaatttcttttacgtttgtcataataaataatctcctttaaatttataattttatttttaataactcgttataattaactttaattgtttgtgtgtgtcgtgttgttgaaagtaatgtatatgtggcaggtaataattctttggcttgttgtttagttaaatgatactcatgtaatgggaaaaatttttcaatatattcatctttatcatcgagataattaagaatataacctttaacacgtaaagtaacaatctcatcaggtttcattatatcactcctttctaaaaaacgtaaacgttatatgtttcataaaaacttttatgcatatcccattgttccaaagcgtcatctgtgatgtatgatagaacagatgttggttcaatttcattgttaagtttgtcgtttaagaacttaacaatagctgtgttatagtttaataacagttgttggcaagctgaaactaaattgatagcattatcaaatgtatatgctggattccattcaattaacttattgaacagttgcaacatttcagtatatgcttgtcctttttcatttggtgcattatctgcttgaatcattttattgttacctccttgttttgattacataacaaatataacatacgtatttaaattagtcaaacgtttttgtgaaaaagttttaaagtttttttaaaataagaaaaatcaatgtatataatgaagaaactagaaaatgcatattgttttaatttaattaaaattaaaatcgaatattttttcggtttaaagaatatttgaatgtttcgaatagatgttattagtagtagcacagattatataagattataaaagaatttataagttgattaataagttatgaattagttaatcgtttattttaaacggtggggtagtgtaaatttacacaaaaaatttccct

SEQ ID NO: 2 - DNA, 366 bp, Staphylococcus phage sp.
  source 1..366: mol_type = genomic DNA; note = MESA-01
atgagtgagtttgaagaaattgttaaatctgatgaagaaactgaagaatcaactgaagaatcaactgaagaatcaactgaagaatcaactgaagaatctactgaagataaaacagtagaaacaattgaagaagaaaatgaaaacaaattagaaccaactacaactgatgaagatagtgcaaaattagaccctgttgttttagaacaacgtattgcttcattagaacaacgatttactaattttgaaacatcacaaatgcaacaaacacaacaagtagaatcaactacaccagatgtaactaatgctgataaagaagacaaagactattcagatgaagaattagtagataagttagatttagactag

SEQ ID NO: 3 - protein, 121 aa, Staphylococcus phage sp.
  source 1..121: note = MESA-01
MSEFEEIVKSDEETEESTEESTEESTEESTEESTEDKTVETIEEENENKLEPTTTDEDSAKLDPVVLEQRIASLEQRFTNFETSQMQQTQQVESTTPDVTNADKEDKDYSDEELVDKLDLD

SEQ ID NO: 4 - DNA, 183 bp, Staphylococcus phage sp.
  source 1..183: mol_type = genomic DNA; note = MESA-01
atgtatgaaggtaataacatgcgttctatgatgggaacatcatatgaagattcacgtttaaacaaacgtacagaattaaatgaaaatatgtcaattgacgttaataaaagcgaagatagttatggggttcaaattcattcactatctaagcaaacatttactggagacgtagaggaggaataa

SEQ ID NO: 5 - protein, 60 aa, Staphylococcus phage sp.
  source 1..60: note = MESA-01
MYEGNNMRSMMGTSYEDSRLNKRTELNENMSIDVNKSEDSYGVQIHSLSKQTFTGDVEEE

SEQ ID NO: 6 - DNA, 1218 bp, Staphylococcus phage sp.
  source 1..1218: mol_type = genomic DNA; note = MESA-01
atggctacaacaaaaaatgaaactgccctattagttgctaaatcagcaaaagcagcattacaagattttaaccatgattattcaaaatcttggacttttggtgataagtgggataatacaaacacaatgtttgaaacatttgttaataaatatttattccctaaaattaatgaaacattattaattgatattgcattaggtaaccgttttaattggttagcaaaagagcaagactttatcggacaatattcagaagaatatgtaattatggatactgttccaattaatatggacttatcaaaaaacgaagaattaatgttaaaacgtaattatccacgtatggctactaagctatatggtagtggtattgtaaaaaaacaaaaattcacattaaacaacaacgacgttcgttttaatttccaaacattagcagacgctacaaattatgcgttaggtgtttataaaaagaaaatttctgatattaatgtattagaagaaaaagaaatgagagcaatgttagttgattactcgttaaatcaattatcagattcaaacatacgcaaaacaacatcaaaagaagatttagcaagtaaagtatttgaagcgattcttaacttacaaaacaacagtgcaaaatacaatgaggtacatcgtgcttctggcggcgctattggtcaatacacaactgtgtctaagttaaaagacattgttattttaacaacagattcattaaaatcttatttattagatacaaaaattgcgaacactttccaaatcgctggtattgacttcacagaccatgttattagctttgatgatttaggtggcgtgtttaaagtaacgaaagaatttaaattacaaaatcaagatacaattgatttcttacgtgcttacggtgattatcaagcacaaatcggtgatactattcctttagatgctgtatttacttatgacgtttcaaaacttaaagagtttacaggtaacgttgaagagattaaaccaaaatcagatttatatgcgtttattttagatattaattcaattaaatataaacgctatacaaaaggtatgttaaaacaaccattctataatggtgagtatgatgaggttacacattggatacattactattcatttaaagctgtaagcccattctttaacaaaattttaattactgaccaagatgttacacctaaaccagatactgtttcagaatag

SEQ ID NO: 7 - protein, 405 aa, Staphylococcus phage sp.
  source 1..405: note = MESA-01
MATTKNETALLVAKSAKAALQDFNHDYSKSWTFGDKWDNTNTMFETFVNKYLFPKINETLLIDIALGNRFNWLAKEQDFIGQYSEEYVIMDTVPINMDLSKNEELMLKRNYPRMATKLYGSGIVKKQKFTLNNNDVRFNFQTLADATNYALGVYKKKISDINVLEEKEMRAMLVDYSLNQLSDSNIRKTTSKEDLASKVFEAILNLQNNSAKYNEVHRASGGAIGQYTTVSKLKDIVILTTDSLKSYLLDTKIANTFQIAGIDFTDHVISFDDLGGVFKVTKEFKLQNQDTIDFLRAYGDYQAQIGDTIPLDAVFTYDVSKLKEFTGNVEEIKPKSDLYAFILDINSIKYKRYTKGMLKQPFYNGEYDEVTHWIHYYSFKAVSPFFNKILITDQDVTPKPDTVSE

SEQ ID NO: 8 - DNA, 984 bp, Staphylococcus phage sp.
  source 1..984: mol_type = genomic DNA; note = MESA-01
atgacaaatagcaaaagaggtttagatgttgaactgtcaaaacaaattaataaaagagtagttgaacatcgtaaccgttttaaacgtcttatgtttaatcgttatttggaatttttacccctactaataaactataccaatcgtgatacggttggtatagattttattcagttagaatccgcattaagacaaaacattaatgttgtggtaggagaagcgagaaataaacaaattatgattcttggttatgttaacaatacttatttcaaccaagcacctaatttttcttcaaactttaatttccaattccaaaaaagattaacaaaagaagatatatattttattgttcctgattatttaatacctgatgaatgtctacaaattcataagcaatacgataattgcatgagtgggaattttgttgttatgcaaaataaaccaatacaatataacagtgatattgaaatcatagaacattacacagatgaattagctgaggttgttttatctcgtttttcattgattatgcaagcaaaattcagcaagatatttaaatcagatattaacgatgaatcaattaatcaacttgtatcagaaatttataacggtgcaccatttgttaagatgtcaccaatgttcaatgcagatgatgatattattgatttaacaagtaattcagttattccagcattaacagaaatgaagagagaatatcagaataaaattagtgaattaagtaactatttaggtattaattcactagctgttgataaagaaagcggtgtttctgatgaagaagcaaaaagtaatcgtggttttactacgtcaaacagtaatatctatttaaaaggacgtgaaccgattacatttttatcaaaacgttatggtttagatattaaaccgtattatgatgatgaaactacgtctaaaatttctatggttgatacactgtttaaagatgaaagtagtggtgaaaatggctag

SEQ ID NO: 9 - protein, 327 aa, Staphylococcus phage sp.
  source 1..327: note = MESA-01
MTNSKRGLDVELSKQINKRVVEHRNRFKRLMFNRYLEFLPLLINYTNRDTVGIDFIQLESALRQNINVVVGEARNKQIMILGYVNNTYFNQAPNFSSNFNFQFQKRLTKEDIYFIVPDYLIPDECLQIHKQYDNCMSGNFVVMQNKPIQYNSDIEIIEHYTDELAEVVLSRFSLIMQAKFSKIFKSDINDESINQLVSEIYNGAPFVKMSPMFNADDDIIDLTSNSVIPALTEMKREYQNKISELSNYLGINSLAVDKESGVSDEEAKSNRGFTTSNSNIYLKGREPITFLSKRYGLDIKPYYDDETTSKISMVDTLFKDESSGENG

SEQ ID NO: 10 - DNA, 825 bp, Staphylococcus phage sp.
  source 1..825: mol_type = genomic DNA; note = MESA-01
atgatgatgaaactacgtctaaaatttctatggttgatacactgtttaaagatgaaagtagtggtgaaaatggctagatataccatgacattatatgattttataaagtcagaactgattaaaaaaggttttaatgaatttgtaaataacaataaattaaccttttatgatgatgaatttcaatttatgcaaaaaatgcttaaatttgataaagatgtacaagcaattgtaaatgaaaaagtatttaaaggtttttcacttaaaaatgagttatcagatttactttttaaaaaatcatttacgattcactttttagatagagaaattaacagacaaaccgttgaagcgtttggcatgcaagtgattacagtgtgcataacacatgaggattatttaaatgttgtttattcatctagtgaagttgaaaagtatctacaagcacaagggtttactgaacacaatgaagatacaacaaacaatactgatgaaacatcgaatcaaaatgctacatcattagacaattcaacaggtatgactgcaaacagaaatgcttttgtgacattacctcaaagtgaggttaatatcgatgttgataatacaacattacaatttgctgataacaataccattgataatggtaaatcagtgaataaatcaagtagtgaaagtaatcaaaacgcaaaacgtaatcaaaatcaaaaaggaaatgctaaaggtacacaattcacaaaacaatacttaatcgaaaatatcgataaagcatatgatttaagaaaaaaaatattaaacgaatttgataaaagatgttttttacaaatttggtaa

SEQ ID NO: 11 - protein, 274 aa, Staphylococcus phage sp.
  source 1..274: note = MESA-01
MMMKLRLKFLWLIHCLKMKVVVKMARYTMTLYDFIKSELIKKGFNEFVNNNKLTFYDDEFQFMQKMLKFDKDVQAIVNEKVFKGFSLKNELSDLLFKKSFTIHFLDREINRQTVEAFGMQVITVCITHEDYLNVVYSSSEVEKYLQAQGFTEHNEDTTNNTDETSNQNATSLDNSTGMTANRNAFVTLPQSEVNIDVDNTTLQFADNNTIDNGKSVNKSSSESNQNAKRNQNQKGNAKGTQFTKQYLIENIDKAYDLRKKILNEFDKRCFLQIW

SEQ ID NO: 12 - DNA, 1941 bp, Staphylococcus phage sp.
  source 1..1941: mol_type = genomic DNA; note = MESA-01
atggcatataatgaaaatgattttaaatattttgaggatattcgtccattcatagatgagatatataaaacaagagaaacatatacaccgttctatgatgatagagctgattataatactaattcaaaatcgtattatgattatgtttcaaaactatcacgactgattgaagtattagcacgtcgtatttgggaatatgacggtgaattaaaaaaacgtttcgaaaattgggacgatttaatgaaacaatttccagatgacgctaaagaattatttagaggatggttaaacgacggcacgattgataaaatcattcatgatgagtttacaaaatattcagctggtctaacttcagcttttgctgtatttaaaattgctgaaatgaaacaaatgaatgattttaaagcagaagttaaagacttaattaaagatattgaccgtttcgttaatgggtttgaattaaatgaacttgaaccaaagtttgttatgggattcggtggaatacgtaacgctgttaaccaatctgttaatattgataaagaaacaaatcatatttatacaacacaatcagactcacaatctccagagggcttttggataaataaattaacccctagtggtgatttactttcaagtatgcgtatcgtacaaggtgggcacggcacaacagtaggtttagaaagacaatcaaacggtgaaatgaaaatatggttacatcatgacggtgtagctaaactattacaagtagcttataaagataattacgttttagatttagaagaggcaaaaggtttaacagattatacaccacaatcacttttaaacaaacatacatttacaccattaattgatgaagcgaatgacaaactcattttacgtttcggagatgggacaatacaggtacgttcaagggtagatgtaaaaaatcatattgataacgtagaaaaagaaatgacaattgataattcagaaaataacgatacacgctggatgcaagggatagctgttgatggggatgatttatactggttaagtggtaatagttctgttaattcacatgttcagattggaaagtattcattgaaaacaggggaaaaaatatacgattatccgtttaaattatcatatcaagatggtattaacttcccacgtgataatttcaaagagccagagggtatttgtatttatattaatccaaaaacaaaacgtaaatcattattacttgcaatgacaaatggcggcggtggtaaaagattccataatttatatggattcttccaaactggagaatatgaacattttagtgccttacgtgcaagaggtgcacaaaactataaattaacaaaagatgatggacgcgctttatcaataccagactatatagatgatttaaacaacttaacacaagcaggattttattatatcgatggtggaacagcaggaaaacttaaaaacatgcctacaaacggtagtaagaaaattattgacgcaggttgctttatcaatgtataccctacaacacaaacattaggaacagtgcaagaaataacacgtttctcaacaggtcgtaaaatggttaaaattattcgaggtatgacgttagatgtgtttacattaaaatgggattatggtttatggacaacaatcaaaacagacgcaccatatcaagaatatttggaggcaagtcaatacaacaattggattgcatatgtaacaacaccgggtgagtattacattacaggtaatcaaatggaattatttaaagacgcaccagatgatattaaacacgttggtgcatggttgaaagtgtcaagtgggaatgctgttggtgaggttagacaaacattagaagcaaatatagctgaatataaagaattctttagtaatgtcaatgcggaaacaaaacatcgtgaatacgagtgggtagcaaaacataaatag

SEQ ID NO: 13 - protein, 646 aa, Staphylococcus phage sp.
  source 1..646: note = MESA-01
MAYNENDFKYFEDIRPFIDEIYKTRETYTPFYDDRADYNTNSKSYYDYVSKLSRLIEVLARRIWEYDGELKKRFENWDDLMKQFPDDAKELFRGWLNDGTIDKIIHDEFTKYSAGLTSAFAVFKIAEMKQMNDFKAEVKDLIKDIDRFVNGFELNELEPKFVMGFGGIRNAVNQSVNIDKETNHIYTTQSDSQSPEGFWINKLTPSGDLLSSMRIVQGGHGTTVGLERQSNGEMKIWLHHDGVAKLLQVAYKDNYVLDLEEAKGLTDYTPQSLLNKHTFTPLIDEANDKLILRFGDGTIQVRSRVDVKNHIDNVEKEMTIDNSENNDTRWMQGIAVDGDDLYWLSGNSSVNSHVQIGKYSLKTGEKIYDYPFKLSYQDGINFPRDNFKEPEGICIYINPKTKRKSLLLAMTNGGGGKRFHNLYGFFQTGEYEHFSALRARGAQNYKLTKDDGRALSIPDYIDDLNNLTQAGFYYIDGGTAGKLKNMPTNGSKKIIDAGCFINVYPTTQTLGTVQEITRFSTGRKMVKIIRGMTLDVFTLKWDYGLWTTIKTDAPYQEYLEASQYNNWIAYVTTPGEYYITGNQMELFKDAPDDIKHVGAWLKVSSGNAVGEVRQTLEANIAEYKEFFSNVNAETKHREYEWVAKHK

SEQ ID NO: 14 - DNA, 750 bp, Staphylococcus phage sp.
  source 1..750: mol_type = genomic DNA; note = MESA-01
atgaagtcattacaacaagcaaaacaatggattgatgtcaatactggtagaggcattgactttgatggcgcatatggatttcaatgtatggatttagctgtagcttatatgtattatgttactgacggaaaagtgcgtatgtggggtaatgccaaagacgcaattgataataactttaaaggtttagctaaggtgtatcaaaatacacctagctttaaacctcaattaggtgatattgctgtttatacaaactcacaatatggtcatattcaagttgtgattagtggtaatttagattattatacatgtctagagcaaaactggttaaatggtggttatgacggttgggaaaaagcaacaatcagaacacattattatgacggtgtaacacactttattcgtccaaacttttcaaatagtaatagtaaagtattagaacaaaacattcaacaaacaaataaatggaaacaaaatcaatacggtacattatataaatctgaaaatggtacatttacatgtggatttttaccaatatttgcacgtgttggaagtcctaaattaagtgaaccgaatgggtattggttccaaccaaacggctatacaccatatgatgaggtttgtttatcagatggactagtgtggattggttataattggcaaggtacacgttattatttaccagtgagacaatggaacggtaaaacgggtaatagttatagcattggtttaccatggggggtgttctcataa

SEQ ID NO: 15 - protein, 249 aa, Staphylococcus phage sp.
  source 1..249: note = MESA-01
MKSLQQAKQWIDVNTGRGIDFDGAYGFQCMDLAVAYMYYVTDGKVRMWGNAKDAIDNNFKGLAKVYQNTPSFKPQLGDIAVYTNSQYGHIQVVISGNLDYYTCLEQNWLNGGYDGWEKATIRTHYYDGVTHFIRPNFSNSNSKVLEQNIQQTNKWKQNQYGTLYKSENGTFTCGFLPIFARVGSPKLSEPNGYWFQPNGYTPYDEVCLSDGLVWIGYNWQGTRYYLPVRQWNGKTGNSYSIGLPWGVFS

SEQ ID NO: 16 - DNA, 930 bp, Staphylococcus phage sp.
  source 1..930: mol_type = genomic DNA; note = MESA-01
atggctgatagaatcgtaagaagtttaagaggtattgattcagtagagaagttaaacgacaatttagtagaagcaaacgacttaatcacaaccaaagacgataacatatatataagacgtgatgaggattattataagctaacatttaaagatgaattattagaaaaaatcaatacaaacacaaaagcgattgataaaaataaaaatgacatcactacaaataaaaaaaatatatctcaaaacgctacagatattattaacattaaagaagataatattcaacaagataaaaaaattaaaaatttatctgacgttcaaacagaacatgaaaatacattaaacaatcatgatgacgcaattcgtttattagatgatgaaaatacaaaaaacaaattagcaattgaaaagaataaacaagatattatcgctacaaaagatacaatcggacaaaataaacaaagtattgaaaacttagcttcaacggtttcaaacaacacgattgaaacaagtaaaaaaattgaatcaactaaaacagaattacaagaacaaatcaaatcttcaaaaacaagtgtgaatgatacaggttggttaaatattcaacttgaaagtggtatcacagcaagtgattcaagtggtggatattccacaccacaatatcgaattatagatataaataacatcagaactattcaattaagaggtgtattaaaaggtgttaagaaaaatggagatattaaattaggcacgattaatgctaatttaaaagtaacacatcactatacacaatgtgctattgattctaaaatgataaacacaagattatatgtaaactttaataacgaattacattttgttacatcaggttatcaagatagtgaactttcttctggagataaacgttttgttattgatacacaaatcattgaataa

SEQ ID NO: 17 - protein, 309 aa, Staphylococcus phage sp.
  source 1..309: note = MESA-01
MADRIVRSLRGIDSVEKLNDNLVEANDLITTKDDNIYIRRDEDYYKLTFKDELLEKINTNTKAIDKNKNDITTNKKNISQNATDIINIKEDNIQQDKKIKNLSDVQTEHENTLNNHDDAIRLLDDENTKNKLAIEKNKQDIIATKDTIGQNKQSIENLASTVSNNTIETSKKIESTKTELQEQIKSSKTSVNDTGWLNIQLESGITASDSSGGYSTPQYRIIDINNIRTIQLRGVLKGVKKNGDIKLGTINANLKVTHHYTQCAIDSKMINTRLYVNFNNELHFVTSGYQDSELSSGDKRFVIDTQIIE

SEQ ID NO: 18 - DNA, 1755 bp, Staphylococcus phage sp.
  source 1..1755: mol_type = genomic DNA; note = MESA-01
atgaggaaattaacaaattttgtatttttttataatacaccttttacagattatcaaaatacaatacattttaatagtaataaagaacgagatgattattttttaaaaggtcgtcattttaagtcattagattattcaaaacaaccgtataacttcattcgtgatagaatggaagttaatgtagatttaagttggcatgacgcacaaggtattaactatatgacgtttttatctgattttgaaaatagacgttattatgcgtttgtgaaccaaattgaatatattaacgatgttacgacaaaaatttattatgtcattgatacagttatgacgttcacacaaggtaatgtgttagagcaactctcaaacgtcaatattgaacgacaacatttatcaaaacgtacgtataactatatgttaccaatgttaagaaacaacgatgatgttttaaaagtaagtaataaaaactatgtttataaccaaatgcaacagtatttgcaaaatgttgtactatttcaatcaagtgctgatttatctaaaaagtttggtactaaaaaagagcctaatttagacacatcaaaaggaacaatatatgacaatatcacatcaccagttaatttgtatgtcatggaatataatgattttattaactttatggataaaatgagcgcatatccgtggattacgcaaaacttccaaaaagtacaaatgttacctaaagactttattaacgaaaaagatttagaggacgttaaaacaagtgaaaaaattactggtttaaaaacattaaaacaaggtggtaaatctaaagagtggagcttaaacgatttatcattaagtttttcaaaacttcaagaaatgatgttgtctaaaaaagatgaattaaaacacatgatacgcaatgaatacatgaccattgaattttatgattggaatggtaatacaatgttacttgacgctggtaaaatttcagaaaaaacaggagtgaaactaagaacaaaatctattattggttatcataatgaagttagagtttatccagtagactataacagcgctgaaaacgatagaccgatacttgctaaaaacaaagacatattaattgatacaggttcatttttaaatacaaatattacatttaatagctttgctcaagttccaattttaattaataacggtatattaggacaatcacaacaagcaaatagacaaaaaaatgcagaaagtcaattaatcacaaatcgaattgataacgtgttaaatggtagtgaccctaaatcaagattttacgacgctgtcagtgttgctagtaatttaagtccaacagctttatttggtaagtttaatgaagaatataatttttataagcaacaacaagctgaatataaagatttagcactacaaccaccgtctgtgacagaatcagatatgggtaatgcattccaaattgcaaataatattaacggtttaacaatgaagattagtgtaccgtctccaaaagaaattacattcttacaaaaatattacatgttatttggttttgaagtgaatgactataacaatttcattgaacctattaatagtatgactgtatgtaattacttaaaatgtaacggaacgtatacattacgtgatattgaccctatgttaatggaacaactcaaagcaatattagaaactggtgtgagattttggcataatgacgggtcaggtaatccaatgttacaaaatccattaaataataaatttagatag

SEQ ID NO: 19 - protein, 584 aa, Staphylococcus phage sp.
  source 1..584: note = MESA-01
MRKLTNFVFFYNTPFTDYQNTIHFNSNKERDDYFLKGRHFKSLDYSKQPYNFIRDRMEVNVDLSWHDAQGINYMTFLSDFENRRYYAFVNQIEYINDVTTKIYYVIDTVMTFTQGNVLEQLSNVNIERQHLSKRTYNYMLPMLRNNDDVLKVSNKNYVYNQMQQYLQNVVLFQSSADLSKKFGTKKEPNLDTSKGTIYDNITSPVNLYVMEYNDFINFMDKMSAYPWITQNFQKVQMLPKDFINEKDLEDVKTSEKITGLKTLKQGGKSKEWSLNDLSLSFSKLQEMMLSKKDELKHMIRNEYMTIEFYDWNGNTMLLDAGKISEKTGVKLRTKSIIGYHNEVRVYPVDYNSAENDRPILAKNKDILIDTGSFLNTNITFNSFAQVPILINNGILGQSQQANRQKNAESQLITNRIDNVLNGSDPKSRFYDAVSVASNLSPTALFGKFNEEYNFYKQQQAEYKDLALQPPSVTESDMGNAFQIANNINGLTMKISVPSPKEITFLQKYYMLFGFEVNDYNNFIEPINSMTVCNYLKCNGTYTLRDIDPMLMEQLKAILETGVRFWHNDGSGNPMLQNPLNNKFR

SEQ ID NO: 20 - DNA, 375 bp, Staphylococcus phage sp.
  source 1..375: mol_type = genomic DNA; note = MESA-01
atgtttatttatgcaggagatttaaaattattatatttcttatttgtattaatgtttatcgacattattacaggtatagcaaaagccattaagaataataatttatggtcaaagaaatctatgaaagggtttgctaaaaaattactcattttttgtattattatactagcaaatatcattgaccaaattttacaattaaaaggtgggttactcatgataacgatattctattatatcgcaaatgagggcttatctattgttgaaaattgtgcagaaatggatgtattagtgccagagcaaatcaaagaacgattaaaagtaataaaaaacgagtctgaaaagagtgataacaatgaacgaccaagagaagattga

SEQ ID NO: 21 - protein, 124 aa, Staphylococcus phage sp.
  source 1..124: note = MESA-01
MFIYAGDLKLLYFLFVLMFIDIITGIAKAIKNNNLWSKKSMKGFAKKLLIFCIIILANIIDQILQLKGGLLMITIFYYIANEGLSIVENCAEMDVLVPEQIKERLKVIKNESEKSDNNERPRED

SEQ ID NO: 22 - DNA, 1440 bp, Staphylococcus phage sp.
  source 1..1440: mol_type = genomic DNA; note = MESA-01
atgaacgaccaagagaagattgataaatttacccactcgtatattaatgatgacttcggtttaacgatagaccaacttgtaccaaaagttaaaccttacgggcgatttaatgtatggctaggtggtaatgaaagtaaaataagacaagtattaaatgcagtaaagtcgataggtgtttcaccaaccctttttgctgtttatgaaaagaatgaagggtatagtgcaggtttagggtggttaaaccatacatctgcacaaggtgattatttaactgatgctaaatttgtagctagaaaattagtatcacaatcaaaacaagcaggacatccgtcttggtatgatgcaggtaacattgtgcattttgtacctcaagacgtacaaagaaaaggtaatgaagattttgctaagaatatgaaagcaggtacagtcggacgtacctatattccattaacggcggctgctacttgggctgcttattacccactaggtctgaaagcttcatataatagagtacaaaactatggtaatccatttttagacggtgctaataccattttagaatggggcggtaaaattgacgggaaaggtggttcacctagtagtggctcatctgatagtggttctgatagtggaggaaattcattactagccctagcaaaacaagctatgcaagaattattaaaaaaagtgcaagacgcattacaatgggacgtacacagtatcggtcatgataaatattttagtaatgattattttacattagaaaaaacgtttaacaatacgtatcatattaaaatgactatagggttacttgattcactcaaaaagattatagatagtattcatattgacagcggtggtagctcatctaatcctactgatgatgacggagatcataaaccaattagtggtaaatctgttaaacctaacggaaaaagtggtcgtgttattggtggtaactggacatatgaccaattacctgaaaaatataaaaaggcaattggtgtacctttgttcaaaaaagaatatttatataaaccgggtaacatattccctcaaaccggtaatgcaggacaatgtacagaattaacatgggcgtatatgtcacaattacatggtaagcgtcaacctacagacgacggtcaaattactaacgggcaacgtgtatggtacgtttataagaagttaggtgcaaaaacaacccataacccaacagtaggttatggtttttcaagtaaaccaccatacttacaagcaagtatttatggtattggacacacaggtgttgtcgttgctgtatttgacgatggttcattcttagttgcaaactataacgtaccaccttacgttgcaccgtcacgagtgttattatatacactaattaatggtgtaccagaaaatgcaggtgataatattgtattctttagtggtattgcttaa

SEQ ID NO: 23 - protein, 479 aa, Staphylococcus phage sp.
  source 1..479: note = MESA-01
MNDQEKIDKFTHSYINDDFGLTIDQLVPKVKPYGRFNVWLGGNESKIRQVLNAVKSIGVSPTLFAVYEKNEGYSAGLGWLNHTSAQGDYLTDAKFVARKLVSQSKQAGHPSWYDAGNIVHFVPQDVQRKGNEDFAKNMKAGTVGRTYIPLTAAATWAAYYPLGLKASYNRVQNYGNPFLDGANTILEWGGKIDGKGGSPSSGSSDSGSDSGGNSLLALAKQAMQELLKKVQDALQWDVHSIGHDKYFSNDYFTLEKTFNNTYHIKMTIGLLDSLKKIIDSIHIDSGGSSSNPTDDDGDHKPISGKSVKPNGKSGRVIGGNWTYDQLPEKYKKAIGVPLFKKEYLYKPGNIFPQTGNAGQCTELTWAYMSQLHGKRQPTDDGQITNGQRVWYVYKKLGAKTTHNPTVGYGFSSKPPYLQASIYGIGHTGVVVAVFDDGSFLVANYNVPPYVAPSRVLLYTLINGVPENAGDNIVFFSGIA

SEQ ID NO: 24 - DNA, 2280 bp, Staphylococcus phage sp.
  source 1..2280: mol_type = genomic DNA; note = MESA-01
atgggattacttgagtgtatgcaatatcataaacatgaacgtaaaatgattttatattgggatattgaaacattagcatataataaaatcaatggacgtaaaaaaccaactaaatataaaaacgtcacgtattcagttgctattggttggtttaatggttatgaaattgatgtagaggtattccctagttttgaagcattttatgattcgttttataagtatgtgaaacgacgtgatacaatcacaaaatcaaaaacagacattattatgattgcacataactgtaataagtatgacaatcattttttacttaaagatacgatgcgttactttgataatattacacgtaaaaatgtgtttttaaaatcagctgaagataatgaacacaccctaaaaatgaaagaagctcaaatattatctaaaagtcaaaatgtcatacttgaaaaacgtgtaaagtcctctattaatttagatttaacgatgtttttaaacggatttaaattcaatatcattgataactttatgaaaaccaatacatcaatcgctacacttggcaaaaagttattagacggtggttatcttacagaaaaccaactcaaaactgattttaactatacaatctttgataaagaaacagatatgtcagacagtgaagcttatgactatgctactaaatgctttacaaaattaacatcagaacaacttacatatattcataacgatgtaattattttaggtatgtgtcatatacattatagtgatatttttccaaattttgattataataaattaacgttttcattaaatattatggaatcatatttaaataatgaaatgacacgttttcaattacttaataaatacgatgacatcgctatttcatatacgcattatcaattccatgatatgaatttttatgattatattaaatcattttatcgtggtgggttaaatatgtataacacaaagtatattaacaaaatcgttgatgaaccttgtttttctattgatatcaattcaagttatccgtatgtgatgtatcatgaaaaaattccaacatggttatacttttacgaacactattcagaaccaacgttaattcctacttttttagatgatgataattattttacgttatataagattgataagcatacatttaaccatgatatattactaaaaataaaatcacgtgtattacgtcaaatgattgtaaaatactataataatgataatgattacgttaatatcaacacaaatacattaagaatgattcaagacattacagatattgattgcacaaaaatacgtgtcaattcatttgtgatgtatgaatgtgaatactttcatgctcgtgatattatatttcaaaactattttattaaaacacaagggaaactcaaaacaaaaattaatatgacttcaccttatgactataaaatcactgatgatattaacgaacacccgtactcaaatgaagaggttatgctgtctaaagtcgttttaaacggtttatatggtattcctgctttacgttcacactttaatttattccgtttagatgaaaacaacgaattatttaatatgataaacggatacaaaaacacagaacgtaatttactattttctacttttgtaacatcacgctcattatataacttattagttccgtttcaatacttaactgaaagtgaaatagacgataatttcatttattgtgatactgatagtttgtatatgaaatccgttgtgaaacccttgttaaaccccgatttattcgacccgattgccttaggtaaatgggatattgaaaacgaacagatagataagatgtttgtactcaatcataagaaatatgcttatgtgacgaacggaaaaattaaaatcgcttctgctggtataccgaaaaacgcttttgatacaagcgttgattttgaaacatttgtcagtgaacaattttttgatggtgctgttgtttacaacaataaaagtatctataatgagcagggtacgatttcgatttacccgtctaaaactgagattgtaagcggagatgtatatgatgatgtcttttcagatgaattaaatgaaaaaagagaatttttgctaaaagacgctagagaaaattttgaacatagtgaatatgatgatattctatatattgaaagtgatattggcacattttcattaaatgatttattcccagtagaacgttctgcaaaatatcgttcaaagttaaatcaattaaaacaaatacatgatgacatacgaaaagggtaa

SEQ ID NO: 25 - protein, 759 aa, Staphylococcus phage sp.
  source 1..759: note = MESA-01
MGLLECMQYHKHERKMILYWDIETLAYNKINGRKKPTKYKNVTYSVAIGWFNGYEIDVEVFPSFEAFYDSFYKYVKRRDTITKSKTDIIMIAHNCNKYDNHFLLKDTMRYFDNITRKNVFLKSAEDNEHTLKMKEAQILSKSQNVILEKRVKSSINLDLTMFLNGFKFNIIDNFMKTNTSIATLGKKLLDGGYLTENQLKTDFNYTIFDKETDMSDSEAYDYATKCFTKLTSEQLTYIHNDVIILGMCHIHYSDIFPNFDYNKLTFSLNIMESYLNNEMTRFQLLNKYDDIAISYTHYQFHDMNFYDYIKSFYRGGLNMYNTKYINKIVDEPCFSIDINSSYPYVMYHEKIPTWLYFYEHYSEPTLIPTFLDDDNYFTLYKIDKHTFNHDILLKIKSRVLRQMIVKYYNNDNDYVNINTNTLRMIQDITDIDCTKIRVNSFVMYECEYFHARDIIFQNYFIKTQGKLKTKINMTSPYDYKITDDINEHPYSNEEVMLSKVVLNGLYGIPALRSHFNLFRLDENNELFNMINGYKNTERNLLFSTFVTSRSLYNLLVPFQYLTESEIDDNFIYCDTDSLYMKSVVKPLLNPDLFDPIALGKWDIENEQIDKMFVLNHKKYAYVTNGKIKIASAGIPKNAFDTSVDFETFVSEQFFDGAVVYNNKSIYNEQGTISIYPSKTEIVSGDVYDDVFSDELNEKREFLLKDARENFEHSEYDDILYIESDIGTFSLNDLFPVERSAKYRSKLNQLKQIHDDIRKG

SEQ ID NO: 26 - DNA, 1248 bp, Staphylococcus phage sp.
  source 1..1248: mol_type = genomic DNA; note = MESA-01
atggtaaaacaaaacaggctagacatggtaagggattatcaaaatgttgtcaatcatgtaagaaaaaagatacctgataattataatcaaattgaattagtcaatgaattaatgaatgatgatttagattactatatttctatttcaaaccgttcagacggtaaatcatttaactatgtatcgttttttatttatttagcaatgaaacttgatattaaatttacattactatcacgtcattacacgttacgtgatgcataccgtgattttattgaggaaattattgataaaaatccattatttaaatcaaaacgtgttgctttcagaagcgcaagagattatttagcgattgtataccaagataaagaaataggtgtgataacagatttaaatagtgcaactgatttgaaatatcattctaactttttaaaacactatcctatcattatatatgatgagtttttagcacttgaagatgattatttatatgatgagtgggataaactcaaaacaatttatgagtccattgatagaaatcatggtaatgtagattatatcggatttcctaaaatatttttacttggtaacgctgttaacttttcaagtcctattttatctaatttaaacatatataatctattacaaaaacataaaatgaacacgtcacgactttataaaaacatttttttagagatgagacgtaatgattatgtcaacgaaaaacgtaatacacgtgcgtttaattcaaatgaggacgctatgacaacaggggaatttgattttaatgaatataatttagctaacgatagtatacggtcacatattaatcaaaacggtgattttttctatatcaaaacagaagataaattcattaaaattatgtataatgtatctacatttatagcaaatattattgtaataccttatacaaaagaatatgaattttgtacgaaaatcaaagatattgatgaaaacgtcacatatcttagagaagatatgttttataaagaaaatatggagcgttattattataatccgtcaaatttacattttgataacgcatattcaaaaaattatgttgttgataatgataggtatttatatttagatgtgaataaaattataaaatttcatattaaaaatgaaatgaagaaaaacatgagtgagtttgaacgaaaagaaaagatatatgaagataactatatagaaaatacaaaacgctacttaatgcaacaatacgaacaataa

SEQ ID NO: 27 - protein, 415 aa, Staphylococcus phage sp.
  source 1..415: note = MESA-01
MVKQNRLDMVRDYQNVVNHVRKKIPDNYNQIELVNELMNDDLDYYISISNRSDGKSFNYVSFFIYLAMKLDIKFTLLSRHYTLRDAYRDFIEEIIDKNPLFKSKRVAFRSARDYLAIVYQDKEIGVITDLNSATDLKYHSNFLKHYPIIIYDEFLALEDDYLYDEWDKLKTIYESIDRNHGNVDYIGFPKIFLLGNAVNFSSPILSNLNIYNLLQKHKMNTSRLYKNIFLEMRRNDYVNEKRNTRAFNSNEDAMTTGEFDFNEYNLANDSIRSHINQNGDFFYIKTEDKFIKIMYNVSTFIANIIVIPYTKEYEFCTKIKDIDENVTYLREDMFYKENMERYYYNPSNLHFDNAYSKNYVVDNDRYLYLDVNKIIKFHIKNEMKKNMSEFERKEKIYEDNYIENTKRYLMQQYEQ

SEQ ID NO: 28 - DNA, 483 bp, Staphylococcus phage sp.
  source 1..483: mol_type = genomic DNA; note = MESA-01
atgcgtaaacgtatgtcagaagcagaccgttggaaaaaagaacgagaagcaagaaaagcagatgaagaaaaaagaaaaaagaatgactttagtggtgtcagttttaaatttgaagatgcatcactacaaaaagaatatattgacgcttgggaatatttttcaaagcttacatttttacctaaagaaaaacatgtgagttatgtaaatgctgtttctttagtacgtggtaaacgacgtgaacaattgttaaaaatattaaacatttatgaacgtaatgatgatagtaataacaaaaatgcaaaaacacataaatatactttatatgatgaaactgctaaagataataatacatcaatgtataaatatatcaaaggtattgaagaattatttgatgaaattggtgaatctaacagaccgaaaacaacaatcaaaaacaaaaaagtcagatataactttttatattatcatttaatagaaaaatga

SEQ ID NO: 29 - protein, 160 aa, Staphylococcus phage sp.
  source 1..160: note = MESA-01
MRKRMSEADRWKKEREARKADEEKRKKNDFSGVSFKFEDASLQKEYIDAWEYFSKLTFLPKEKHVSYVNAVSLVRGKRREQLLKILNIYERNDDSNNKNAKTHKYTLYDETAKDNNTSMYKYIKGIEELFDEIGESNRPKTTIKNKKVRYNFLYYHLIEK

SEQ ID NO: 30 - DNA, 168 bp, Staphylococcus phage sp.
  source 1..168: mol_type = genomic DNA; note = MESA-01
atgaataacctacttaatataattatcatttttcttttagcattattgattacacttgtcattcttatgaccatgcatatacgtgtgtcatttggtgtactatttacaacactaatcatattctatattatctttttaatggttatttatgctatatatggaggttga

SEQ ID NO: 31 - protein, 55 aa, Staphylococcus phage sp.
  source 1..55: note = MESA-01
MNNLLNIIIIFLLALLITLVILMTMHIRVSFGVLFTTLIIFYIIFLMVIYAIYGG

SEQ ID NO: 32 - DNA, 417 bp, Staphylococcus phage sp.
  source 1..417: mol_type = genomic DNA; note = MESA-01
atgaaaataaaacaatcatttaatttatatgacttactttgctacattaacgataacagaaaaaattttcaagataaaaaaatacaatttatttctgatagtggttataattttgtaggtattgatagcgatgatgatatttgtgttaatttttctgaaagtcataatacggacttaacaattaacaatgatttattcacgttagatattgatattgatattaaaaaacatgttttttatttacttgagtattattatatagattctgtaaataacacatttgtaagagaaacttcatatggtgttacaatagacgatgtattagtaaattttgattgtccactcgaaagcggattaatgattatttaccaaaacaaagttatatataaaaacggaaatgtgatagaacatgaataa

SEQ ID NO: 33 - protein, 138 aa, Staphylococcus phage sp.
  source 1..138: note = MESA-01
MKIKQSFNLYDLLCYINDNRKNFQDKKIQFISDSGYNFVGIDSDDDICVNFSESHNTDLTINNDLFTLDIDIDIKKHVFYLLEYYYIDSVNNTFVRETSYGVTIDDVLVNFDCPLESGLMIIYQNKVIYKNGNVIEHE

SEQ ID NO: 34 - DNA, 174 bp, Staphylococcus phage sp.
  source 1..174: mol_type = genomic DNA; note = MESA-01
atgaaaaaatataaaacagtcttattatatactgacacaatagaaaatgattttcatcatcaaatattaatgtttgaagatttgtataacgccaaagttttatactcttattatgaattcaatccatttcataataattatgcgtatgttatagaatatatagaggagacttaa

SEQ ID NO: 35 - protein, 57 aa, Staphylococcus phage sp.
  source 1..57: note = MESA-01
MKKYKTVLLYTDTIENDFHHQILMFEDLYNAKVLYSYYEFNPFHNNYAYVIEYIEET

SEQ ID NO: 36 - DNA, 369 bp, Staphylococcus phage sp.
  source 1..369: mol_type = genomic DNA; note = MESA-01
atgacaaacgtaaaagaaattttatctagacaccaaaacacattagcgagatttgaatttgaggaaaaagaaagagaatttatcaaattatcggaattagttgaaaaattcggaatcaaacaagaatacatcgttagagcattatttacaaacaaagaatcaaaattcggtgaacaaggtgttattgtaacagatgattataatgttaacttaccgaatcacttaactgaattaattaaagacatgcgtcaagatgaagatgttgtagacattattaataaaggtgaagttgtgttctcaatttatgaatacgaaaacaaaaaaggtcaaaaaggatattctattaactttagtcaagtatcattttaa

SEQ ID NO: 37 - protein, 122 aa, Staphylococcus phage sp.
  source 1..122: note = MESA-01
MTNVKEILSRHQNTLARFEFEEKEREFIKLSELVEKFGIKQEYIVRALFTNKESKFGEQGVIVTDDYNVNLPNHLTELIKDMRQDEDVVDIINKGEVVFSIYEYENKKGQKGYSINFSQVSF

SEQ ID NO: 38 - DNA, 225 bp, Staphylococcus phage sp.
  source 1..225: mol_type = genomic DNA; note = MESA-01
atgaaacctgatgagattgttactttacgtgttaaaggttatattcttaattatctcgatgataaagatgaatatattgaaaaatttttcccattacatgagtatcatttaactaaacaacaagccaaagaattattacctgccacatatacattactttcaacaacacgacacacacaaacaattaaagttaattataacgagttattaaaaataaaattataa

SEQ ID NO: 39 - protein, 74 aa, Staphylococcus phage sp.
  source 1..74: note = MESA-01
MKPDEIVTLRVKGYILNYLDDKDEYIEKFFPLHEYHLTKQQAKELLPATYTLLSTTRHTQTIKVNYNELLKIKL

SEQ ID NO: 40 - DNA, 300 bp, Staphylococcus phage sp.
  source 1..300: mol_type = genomic DNA; note = MESA-01
atgattcaagcagataatgcaccaaatgaaaaaggacaagcatatactgaaatgttgcaactgttcaataagttaattgaatggaatccagcatatacatttgataatgctatcaatttagtttcagcttgccaacaactgttattaaactataacacagctattgttaagttcttaaacgacaaacttaacaatgaaattgaaccaacatctgttctatcatacatcacagatgacgctttggaacaatgggatatgcataaaagtttttatgaaacatataacgtttacgttttttag

SEQ ID NO: 41 - protein, 99 aa, Staphylococcus phage sp.
  source 1..99: note = MESA-01
MIQADNAPNEKGQAYTEMLQLFNKLIEWNPAYTFDNAINLVSACQQLLLNYNTAIVKFLNDKLNNEIEPTSVLSYITDDALEQWDMHKSFYETYNVYVF

SEQ ID NO: 42 - DNA, 174 bp, Staphylococcus phage sp.
  source 1..174: mol_type = genomic DNA; note = MESA-01
gtgctactactaataacatctattcgaaacattcaaatattctttaaaccgaaaaaatattcgattttaattttaattaaattaaaacaatatgcattttctagtttcttcattatatacattgatttttcttattttaaaaaaactttaaaactttttcacaaaaacgtttga

SEQ ID NO: 43 - protein, 57 aa, Staphylococcus phage sp.
  source 1..57: note = MESA-01
VLLLITSIRNIQIFFKPKKYSILILIKLKQYAFSSFFIIYIDFSYFKKTLKLFHKNV

SEQ ID NO: 44 - DNA, 17639 bp, Staphylococcus phage sp.
  source 1..17639: mol_type = genomic DNA; note = MESA-05
gtatttaaaatctaatttcttctattaaatagtatttttaaattatttaaactttttaagaaaaaacattgacaaaacttttaaacgtttgctatactatgtatgtaatcaaatcaaggaggtaacaaaatggaaaatgtacaacctttacaaacttacattttaaaaaatggtaatatagttattgttatgggtaatacatcaagttatgaattaacattatttgtacttgaaaaggatgattttgaacaattagaccataattcacctaaaaaaatcgttgatgaaataaggaatgaaatcaatgatttaaggagtgataataatggttaatgttgataacgcacctgaagaaaaagggcaagcctatactgaaatgttgcaactattcaataagttgattcaatggaatccagcttatactttcgacaatgctattaatttagtatcagcttgccaacaactattattaaactataatagttctgttgttaaattcttaaatgatgaactaaacaacgaaactaaaccagaatctattttatcttacattgctggtgatgacccaatagaacaatggaatatgcacaaaggattttatgaaacgtataacgtttacgttttttagaaaggagtgatataataatgaaacctgatgatattgttacattacgaataaaaggttatatttttcattatttagatgataaaaatgaatatgttgaagaatttatcccacttcacgagtatcatttatcaaaatcacaagctaaagacttattacctaatacacacaaacttttatccactacacgacatactaaaactatgcaagtttattacaatgatttactacaaattgcaattgcagaaagcaaataatttaaataagaggagaaataaaaatgacaaacgtaaaagatattttatcaagacaccaaaacacattagcgagattcgaatttgaggaaaaagaaagagaatttatcaaactatcagaattagtagaaaaatacggtatgaaaaaagagtatatcgttagagcattattcacaaacaaagaatcaaaattcggtgagcaaggtgttatcgtcactgacgactataacgtaaacttaccgaatcacttaactgaattaattaaagaaatgcgtcaagacgaggacgttgttaacattatcaacgctggcgaagttcaattcacaatttatgagtatgaaaataaaaaaggtcaaaaaggttactcaatcaactttggtcaagtatcattttaataaaaatttcataggggatatttatcccctatttttatgaggtgcttaaatggaatgtaaatacaaaacggtattattatattgtgatgagattaaaggaaatttaccacatcaaatagcaatgtttgaagaattatatgacgctaaagttgtatattcatattatgaatataatccatttactgaaaaatatgcgtatatgatagaatacattaaggagatataaaatgaaaattacaacaacattaaatacaaaaaacaataatgatttatttactttagatattgacattgatattcatacacattgttttgattttcttattgttttttatagaatatattcaaatccaaattataaaagagaagcatttacagattgcaccattgatgacgtattagaatattttgagaatccattattagctgatattacaataatttataaaaataaggtaatttatgatttaggaaaagtgattgaccatgaataacctattagatattattattgttttccttttagcatttttaattacacttgtaatacttatgacaatgcatatacgtgtgtcatttggtgttttatttactacattgattatattctacattatctttttagtggttatatatgggttatatggaggtcgttagcaatggttagacatacgtccgaaatggataaatggaaaaaagaaagagacgcaagaaaagagcaggaaaaagaattgtttttaaatgattttagtactgttaattttaaatttgatgataaagatttacaagaggcgtacatagacgcatggaaacatttcgcacatttgccctattttccaaaagaaagaaacgtgtcatatgtaaatgctgtatcattggtaagaggtaaaagacatgaacaattaaactatatacttgaaatatataaccgtaaagatgaatctaataataaaaacgctaaaaaacataaatatgctttatatgaattacaagctaaaaataataattcttctatgtataaatatattaaagaaattgacactttatacaaagaaattggtaaatcagacagaccagtgactactattgatgatgaagatgtgaggtataactttttatattatgcaacatttgaagactaattttaatactgtaaacgacatcataaactattataaggagcaaaaacatggtgaaacaaaatcgtttagacatggtaagagattatcaaaatgctgtcaatcatgtcagaaaaaaaataccagataactataatcaaatagaattagttgatgaactcatgaatgatgatatagactattacatatctatttcaaaccgttctgacggaaaatcgttcaactatgtttcattctttatttatttagctataaaacttgatattaaatttactttattatcacgtcattatacattacgtgacgcttatcgtgattttattgaggaaatcatagacaaaaacccactattcaaatctaagcgtgtcacattcagaagcgctagggactatttagctatcatctatcaagataaggaaatcggtgtgattacagatttgaatagtgcaactgatttaaaatatcattctaactttttaaaacactaccctatcattatatatgatgaatttttagcacttgaagatgactatttaattgacgagtgggataagttaaaaacaatttatgaatcaatcgaccgtaaccatggtaatgttgattatatcggcttccctaaaatgtttttactaggtaatgctgtcaacttttcaagtcctatattatccaatttaaatatatataacttattacaaaaacataaaatgaacacatcacgactttacaaaaatatttttttagaaatgcgaagaaacgattacgtaaatgaaaagcgtaacacacgggcattcaattcaaacgatgacgctatgactactggcgagtttgaatttaacgaatataatttggcagatgataatttaagaaatcatatcaatcaaaacggtgattttttctatattaaaactgacgataaatatataaaaattatgtataatgttgatacatttaatgctaatattattgttataccttatacaaaacaatatgaattttgtactaaaatcaaagatatagatgacaatgttatttatttaagagaagatatgttttataaagaaaacatggagcgttattattacaatccaagcaatttacattttgataacgcttactcaaaaaattacgttgttgataatgatagatatttatatttagatatgaataaaattataaaatttcatataaaaaatgaaatgaagaaaaatatgagtgaatttgaaagaaaagaaaaaatatatgaagataactatattgaaaatacaaagaagtatttaatgaaacaatacggcttataaaaggtgtgtaagattatgggattactagaatgcatgcaatatcataaacatgaacgtcgaatgattttatactgggatatagaaacattagcgtacaataaagttaacggacgaaaaaaaccaaccaaatataaaaacgtaacgtattcagtagcaattggttggtttaatggttatgagattgatgtagaagtatttcctagttttgaatcgttttatgacgcattttatacgtatgtgaaacgacgtgatacaatcactaagtcaaaaacagatattatcatgattgcacataactgtaataagtatgacaaccattttttacttaaagataccatgcgttattttgataatatcacacgtgaaaatatatatttaaaatctgcagaagaaaacgaacacacactaaagatgcaagaggctactattttagctaaaaatcaaaatgtgattttagaaaaacgtgtgaaatcgtctattaatttagacttaacaatgtttttaaatggatttaaatttaatattattgataactttatgaaaacaaatacatcaattgcaacattaggtaaaaagttgcttgacggtggttatttaacagacgaccaacttaaaacagattttaattacacgatatttgataaagataacgatatgtcagatagtgaagcctatgactatgctgttaagtgttttgctaaactcacacctgaacaacttacatacattcataatgacgtgattatattaggtatgtgccatattcattatagtgatatatttccaaattttgactataacaaattaacattttcattgaatattatggaatcgtatttaaataatcaaatgacgcgttttcagttactcaatcaatatcaagatattaaaatatcatatacacattaccatttccatgatatgaatttttatgactatatcaaatcattttatcgtggtggtttaaatatgtataacactaaatacataaacaaacttattgatgagccttgtttttctattgatatcaattccagttatccttatgtgatgtatcatgagaaaattccgacatggttatacttttatgaacattattcagaacctacattaatccctacttttttagatgatgacaattatttttcattatataagattgataaagatgtatttaacaatgatttattaattaaaatcaaatcacgtgtattacgtcaaatgattgtaaaatactataataatgataatgattacgttaatattaatacaaatacattaagaatgattcaagacattacgggtattgattgtacgcatatacgtgttaattcgtttgtgatatatgaatgtgaatactttcatgcacgtgatattatatttcaaaactattttattaaaacacaaggtaagttaaaaaacaaaatcaacatgacatcaccttacgactatcatattactgatgacatcaacgaacacccttactcaaatgaggaggttatgttgtctaaagttgtacttaacggattatatggcatacctgcattacgttcacattttaacttattccgtttagatgataacaatgaactatacaatattattaacggttacaaaaacacagaacgtaatattttattttctacatttgtaacatcacgttcattgtataacttattagtaccattccaatacttaacggaaagtgaaattgacgacaattttatttattgtgatactgatagtttgtatatgaaatccgttgttaaacccttattgaaccccagtttattcgacccgatagccttaggtaaatgggatattgaaaacgaacagatagataagatgtttgtactgaatcataaaaaatatgcatatgaagtgaatggaaagattaaaattgcttctgctggtatacctaaaagcgcctttgatacaagcgtcgattttgaaacctttgtacgtgaacaattctttgacggtgcgattatagaaaacaataaaagtatctataatgaacagggtacaatatcgatttatccgtctaaaacagaaattgtatgtggtaatgtatatgatgaatatttttctgatgaacttaatatgaaacgtgaatttatattaaaagacgctagagaaaattttgaccatagtcaatttgatgatattctttatattgaaagtgacattggttcattttcacttaacgacttatttcctgttgaacgttcagttcataacaaatctgatttgcatatattaaaacgcgaacatgatgaattaaaaaaaggcaactgttaaataacagttgcctttttcttttgagataacatgaaaaatgtgtacgaaaattgattatgttttgtattttatttactagcattactagcatgtgttcattatagcatagttaattaagcaataccactgaagaatacaatattatcaccagcgttatgaggtacaccattaatcaatgtatataatacaacccgtgacggtgcgacgtatggtggtacgttatagtttgctactaagaatgaaccatcgtcaaatactgccactacaacacctgtgtggccaataccataagctgttgcttgtaagtatggtggtttacttgaaaaaccataaccaacagtagggttgtgtgttgttttagcacctaactttttatagacgtaccatacacgttgaccgtttgtcacttgaccgtcgtctgtcggttgtcttttaccatgtaattgtgacatatacgcccatgttaattctgtacactgacctgcattacccgtttgaggaaatatgttacctgttttgtataaatattcttttttgaataaaggtacaccaattgcttttttatatttttctggtaactgtgcgtatgtccagttaccaccaatgacgcgaccacttttaccattaggtttgactgatttaccactaattggtttatggtctccgtcatcatcagtagggtttgaactactacccccactatctacttgcacgctatcaatcagtttttttaatgaatcgagtagcccaatcgtcattttaatatgatatgtgttgttaaatgttttttgtaatgtaaaataatcattactaaaaaatttgtcgctacctatactgtgtacatcccattgtaatgcgtcttgtactttttttaataattcttgcatggcttgttttgctaaagcgagcagtgaactaccactgtcaccactactaccactgtcagacgaatcactaggtgaaccacctttaccgtctaatttaccaccccacgcaagaatagtattcgcaccgtctaaaaaaggattaccatagttttgtactttattatatgacgctttcaaacctaggggataatatgccgcccaagtagccgctgctgttaatggaatatacgcacgtccgattgtacctgctttcatatttttagcaaaatctgcattaccttttctttgtacgtcttgcggtacaaaatgcactggattaccgtaatcataccaagacggttgtcctgcttgttttgattgtgatactaattttcttgcaatgaatttagcgtctgttaaatagtcacctcgtgcagatgtatggttcaaccaacctaaacccgcgctatacccttcatttttttcatatacagcaaaaagagtaggtgacacacctatctcttttactgcttttaatacttgtctgattttactttcattaccacctagccatacattaaagcgtccatacccttttactttagggactaactggtctatcgttaaaccgaaatcatcattaatatacgaatgcgtaaatttatctatcttctcttggtcgttcattattatcactcttttcagaatcatttttaattactcttaatttatctttaatttgttctgggactaacacgtccatttctgcacaattttctacgatagataagccctcattagcgatataatagaaaatcgtaatcatgagtaagccaccttttaattgtaaaatctggtcaatgatgttagctaaaatgataatacagaatatcaataattttttagcaaaacctttcattgattttttagaccatagattattatttttaatagctttagcaaaacctgtgataacatcaataatcattaaaacaaataaaaagtatagtaattttaaatccccagcatatataaacatgtgaaacgcttctgagtctgtaaatctgaattttacttcgttcatattataccccctctctaaatttgttatttaatggattttgtaacattggattacctgaaccgtcattatgccaaaatctcacaccagattctaaaattgcttttaattgttccattaacatggggtcaatgtcacgtatggtatacgtacctgtacattttaaatagttgcaaacagtcatactgttaattggttcaataaatgaattatagtcattcacttcaaaaccaaacaacatataatatttttgtaaaaatgtaatttctttaggtgacggtacactaattttcatcgttaaaccgttaatactgtttgcaatttggaatgcgttgcccatttccgactctgtcactgacggtggttgtaatgctaaatctttatattctgcttgttgttgtttgtagaaattatattcttcattaaacttaccaaataaagcagtcggacttaaattacttgctacacttactgcgtcataaaaacgtgattttgggtcactaccatttaatacattatctatacgacttgtgattaattgactttctgcattacgctgtctattggcttgttgtgattgtcctaaaattccgttattaattaatataggcacttgcgcaaaactattaaatgttatatttgtatttaagaatgaacctgtatcaattaatatatctttattttttgcgagaataggtctatcgttttcagcactgttataatcaactggatatactcgcacttcattatgataaccaatgattgactttgtacgtaacttaacacctgttttttgtgaaattttaccagcgtcaagtaacattgtgttaccgttccagtcataaaattcaatcgtcatgtactcattacgtatcatatgtttaaactcgtcttttttagacaacatcatctcttgaagctttgtgaaacttaatgataaatcgtttaaactccattcttttgattttccaccctgttttaacgtctttaatccagtaattttttcacttgttttaacgtcctctaaatcttttgtattaataaaatctttaggtaacatttgaaccttttgaaagttttgtgtaatccatggatatgcactcattttatccataaagttaataaagtcaccatattccataacgtataagttgactggtgatgtgatattgtcatatatcgtacctttagacgtatctaagtttggctcttttttagtaccaaatttctttgataaatcagcgcttgactggaataacactaaattttccaaatactgttgcatttggttatacacatagtttttatttgatacttttaacacatcatcattgttacgtaacattggtaacatatagttatacgtgcgttttgataagtgttgacgttcaatattaacgtttgagagttgctctaatacattaccttgtgtatacgtcataatagtatcaatcacaaaatatatttttaccacaacatcattcacgtattcgatttgattcacaaacgcataatatcgtctgtcctcaaaatctgataaaaacgtcatgtagttaatcccttgcgcgtcatgccactgcatatcaacattgatttccattctatcacgtataaaattatacggttgtttggaatagtctaatgatttaaaatgacgtccatttaaaaaataatcatcacgctctttattactattaaaatgaatcgtattttgataatcagtaaacggtgtgttatagaaaaatttaaaatttgttaattttctcatttttacctccataaaaaatagtcgtataaataatttatacgactattataacatttttattcaatgatttgtgtatctattgcaaaacgtttatcaccatttgaaagctcattatcgctataatttgatgtggtaaaatgtaattcattattaaagtttaaatataatcttgtattcatcattttattatcaatcgcacattgtgtgtagtgatgtgttgattttaagtttgcgttaatcgtacctaatttaatatcaccgtttttcttaatcccttttaataccccttttaattgtatggttttaacaccattaattgttacaatacgatattgcggtgcgggataacccccgttgctatcacttgcaacaataccactttcaagttgcacatcttgccaacctgtatcattaaattgtaattttgcactgttgattttttgatcaaaattttgttcaatttctgatttagattgtgcaattttagtatctatatttaagttatccacttttgttgataaaacgcttatacttgattcgtttgttgatgttttttgttttaaatcactaatatcattatcatattttgataaatcaatatgaccgtttccgtcaacaactggtctatcttttaattcttgaataactttttcaatttttgatattttatcattatttgtttttaaagtagcgatgtcagatttaattgaattaatatcctgtgtataatctttttggttaatactgtttaattttgtttctaactcttttatcttatcaaggttggcatttgctttgatgtcattaatggttttttctaaactttcaagtttcccagcacttgaattttgttttaacttattaatatcctcttgtatttcttgaattgtgttatcattgttttgtttagattttaataattcaatttgactttctatgtcttttattttatcaagattaacactttttttaacttcagctaattctctttttaatgcgtcaatgttttctgcaccagtatttgatttaattatattaatttcatttgtgtttcgtcctacactatctttaatagcgtttaacgattgtgtaagtgtttcatatttatcatcatactttaacattgatttttcaaattcatttaattgtgtttgatgttgttctaaactttcatttaatatgacatctttttcttttaatgaatcaatgtcttttttattactatcaatcattggttggaatttttttattttctctagaaatgttgatattttttctttgttatcattaattgcaatatcatgatgttctatttgaccactgtatttattaatcaatgtttttaaatcattataaaatgttaatttataataaccagtatcagtacggacataaatatgtccgtctgatgtactgattaagtcattttcttctgttaaaaaatctgctaacctgtctattgtttcaacttgtcttaaacttcttacgattctatcagccattgttcacacctcttatttgtatcgtttccaactaaactcaaagaaaaaacctaaaatacccattatgagaacaccccccaaggaataccaacactataactattacccgttttaccgttccattttcttactggtaaataataacgtgtaccttgccagttgtaaccaatccatacataaccatctgataaacaaacttcgtcatatggtgtataaccatttggttggaaccaatatccattaggttcacttaatttaggactacctacacgtgcaaatattggtaaaaatccacatgtaaatgttgcattttcatttctgtaatatgtgccatattggttttgtttccattttccgaatgtgtttacttttgatgtttctaattcgttgctgttactagcagaaaatttaggtctaataaagtgtgttacaccgtcataataatgtgttcttattgttgctttttcccaaccgtcaaacccaccacctaaccagttttgctctaaacatgtataataatctaaattaccacttaacacacattgaatatgtccatattgtccatctgtatatacaccaacatcccctaattgtggtttaaagttcggtgtattttcatacaccattgctaaacctttaaagtcattattgattgcgtctttagcgttaccccacatacgaactttaccgtctgtaatgtaatatatataagcaacagctaagtcagcgcattgataaccaaatgctaaatcaaagtccacacctacaccctcatgcttatatatccattcttttgcttgttgttgtgatttcatgaaacattatcctttcttgacccaactatattgacgtttttttgtatctgcattaacgttactaaagaattctttatgttcggacacattagcctctaatgtttgtcttacttcaccgacagcgttaccacttgaaacacgtaaccatgcacccacttttttaatttcttctggtgcgtctctaaacaattccatttggttaccagtaatataatattcaccaggtgtcgtaacgtaagctatccagttattgtattgacttgcttctaaatactcttgatatggtgcgtctgttttgattgttgtccataaaccataatcccattttaatgtaaatacatcgagcgtcataccacgcatgattttaaccatttttcgaccagtagaaaaacgcgttaattcttgtactgtacctaatgtttgtgttgtagggtatacattgatgaaacaaccagcgtctataatttttttactaccattcattggcatatttttaagtttactagctgtaccaccgtcaatataatagaaccctgcttgtgttaagtcatttaagtcgtcaatatggtctggaattgataacgcacgtccatcgtcttttgttaatttatagttttgtgaacctctcgcacgtaatgcttcaaagtgttcatactcaccaagttgaaagaacccatataaattattgaatcgttttccaccaccgccattagtcatagcaagtaataacgatttacgttttgtttttggattggtataaatacaaataccctccggctctttaaagttatcacgtggaaaattaataccgtcttgatatgataatttaaacggataatcataaatcttttgaccagttgttaatgaatatttaccaatttgaacgtgtgaattaactgaactgttaccacttaaccagtataaatcatcaccatcaacggcaatcccttgcatccatcgattatcattgttttctgaattatcaatcgtcatttctttttctacattatcaatgtgattttttacgtcagctcttgaacgtacctgtattgtcccgtcaccgaatcttaaaacgattttgtcattttcttcatcaattaacggtgtaaacgtgtgtttgtttaaaagtgactgtggtgtataatctgttaaccctttagcctcttctaaatctaaaatataattatctttatatgcgacttgtaacagttttgctacaccgtcatgatgtaaccatattttcatttcaccgttagattgtctttctaaacctattgtagtaccgtgaccaccttgtacaatacgcatactagaaattaagtcaccgctaggcgttaatttattaatccaaaaaccttctgggttttgtgaatctgattgtgtagagtacatttgattcgtttctttatctatgtttatagattggttaacagcattacgaataccaccgaatcccataacaaactttggttcaagctcatttaattcaaaaccattaacaaaacggtcaatgtctttaattaagtctttaacttctgctttaaaatcattcatttgtttcatttctgcaactttaaataatgcaaatgcagatgtaagaccagtactatatttagtaaattcatcatgaataattttatctattgtaccgtcgtttaaccaacctctaaataagtctttcgcttggtctgggaatgctttcattaagtcgtcccaatttttgaaacgtttttttaattcattgtcatagtcccaaatacgacgtgctaatacttcaattagttttgataatcttgaaatataatcataatatgattttgaattggtattataatctgctctatcatcgtaaaacggtgtgtaacgttctctcgttttatatatttcgtctaaaaatggacgaatgtcgtcaaaatatttaaaatcgttttcattatatgccataattttccacctttaccaaatttgtaaaaaacattttttatcaaattcatttaaaattttctttcttaaatcgtatactttatcaatattatcaattaaatactgttttgaaaattgtgtacctttcgcattacctttttgattttgattacgttttgcgttttgattactttcgttacttgatttattcacagttttaccgttgtcaattgtattattatctgcaaatcgtaacgttgtgttatctacatctatgttaacctcgctttgtggtaatgacacataagcatttctgtttgctgtcatgccagttgaattgtctaaagatgttgcattttgatttgaagtttcatcagtgttgcttattgtatcttcgttgtgttctgtgaagccttgtgattgaagatatttttctacttcacttgatgaataaaccacattcaaataatcctcatgtgtgatacatacagtaatcacttgcatgccaaaagcttcaactgtttgtctgttgatttctctatctaaaaagtgtattgtaaatgattttttaaaaagtaagtcagataagtcatctttaagtttaaaacctttaaacactttttcattaacgatggctaaaacgtctttgtcaaacttcaacattttttgcatgaattgaaattcatcatcataaaacgttaatttatcattatttacaaattcattaaaaccttttttaattaattctgatttaataaaatcaaataaagtcattgtatatctagccattgtattcactactttcatcattagacaatgtatctatcattgtgatttctgatgtaacttcatcgtcgtaatacggtttaatatctaaaccatatcgttttgataagaatgtaataggttcacgaccttttaaataaatattactatttgatgtggtaaaacctcggttgctttttgcctcttcgtcagaaacaccgctttccttatccactgctagtgaattaatacctaaatagttacttaattcactaattttattttggtattcacgtttcatctcagttagtgctggaatcacactattactggttaaatctatgatatcatcatcggcgttaaacataggtgacattttaacaaatggtgcaccgttgtatatttccgatacaagttgattaactgactcatcattaatgtctgatttaaatattttgctaaattttgcttgcataattaatgaaaaacgagataaaacaacctcagataattcatcagtataatgttctataatttctatatcactattatattgaatgggtttattttgcataacaacaaaattaccactcatacagttatcatatattttatgaatctgtaagcattcatctggtattaaatagtctggaacaatgaaataaatatcatcttttgttaatcgtttttgaaattgaaaattgaaatttgatgaaaagtttggggcttgattaaaataagtgttgttaacatagcccaaaatcataatttgttcatttcttgctttaccaacaacaacattaatgttttgccttaatgcagattctaattgaataaaatctataccaaccgtatcacgattggtatagtttattaataggggtaaaaattccaaataacgattaaacataagacgtttaaatctgttgcgatgttctacaacacgtttattgatttctttagataattcaacctgtacgcctttattatgtttagtcatttatatcacctctattattctgttctcggtgttacatcttggtcagtaattaaaattttgttaaagaatgggctaatggctttaaatgaatagtaatgaatccagtgtgtaacttcatcaaattcaccattatagaatggttgttttaacatgccttttgtgtaacgtttatatttaattgaattaatatccaaaataaaagcgtataaatctgattttggtttaatttcttcaacactatctttaaattcagatagttttgatacatcgtaagtgaatactgaaccaactggaattgtatcacctttatgagtttgataatcaccgtaagcacgtaagaaattaactgattcatcacttgatacgacaatatctttagtgactttaaaaacaccacctaaatcgtcgaaactaataacatggtctgtaaagtcaatccctgctacttggaaagtgttagcaatctttgtatctaaaagataagattttaagctatcagtcgttaaaataacaatatcttttaattttgaaactgttgtatattgtccgattgcaccacctgaagcacgatgaacttcattatatttagtgctgttgttttgtaagttaagaatagcttcaaaaactttacttgctaaatcttcttttgatgttgctttacgtacatttgattcagataattgattcaatgagtaatcaactaacattgcacgcatttctttttcttctaatacgttaatatcagaaattttctttttatagacacctaatgcataattagttgcgtctgctaatgtttggaaattgaaacgtgtgtcattgttatttaatgtgaatttttgtttcttcacaataccactaccatataacttagtagccatacgtgggtaattacgtttcaacattaattcttcatttttagataagtccatgttaataggtactgtatccataattacatactcttcactgtattgacctataaagtcttgctctttagctaaccaattaaaacggttacctaatgcaatatcgattaataacgtttcattaatcttagggaataaaaatttatttacaaatgtttcaaacattgtattagaattatcccacttatcgccaaacgtccatgattttgaataagtatgattaaagtcttgtaatgcagatttagctgactttgcaactaatagtgctgtttcgttttttgtactcttttctgccatgatttattattcctcctctacatcgccagtaaatgactgttttgaaagtgaatgaatttgtacaccataactatcttcacttttgtttgtatcaattgacatattttcatttaattctgttcgtttatttaatcttgaatcttcatatgatgttcccatcatagaacgcatattgttaccctcatacatgtttaaattcctcctaatctaaatctaacttatccactaattcttcatctgaatagtctttgtcgttgtcatctgcttcagtaacatctggttttggttgtggttgtggttgtggttgttgcatttgtgatgtttcaaaattagtaaatcgttgttctaatgaagcgatacgttgttctaaaacaacagggtctaattttgcactatcttcatcagtaatagtaggttctaatttgttttcattttcttcttcgattgtttctacttttttatcttcagtagattcttcagttgattcttcagttgattcttcggttgattctgaagtttcttcagttgattctgaagtttcttcagttgattctgaagtttcttctttgtcgtctggttttacgatttcatcaaattctgtcattttgacacctccaaatattttataactaattatatcatagaatatttaaataagtaaattaaatttattatatgtta

SEQ ID NO: 45 - DNA, 18007 bp, Staphylococcus phage sp.
  source 1..18007: mol_type = genomic DNA; note = MESA-11
cacttatcatttctttactaaacttataaacactgttcaccaactttcccaacttatctaacctattacatattaattaattacatttattatacatctattgacttttatcaaaagttatgatttgtatttaaaatctaatttcttctattaaatagtatttttaaattatttaaactttttaagaaaaaacattgacaaaacttttaaacgtttgctatactatgtatgtaatcaaatcaaggaggtaacaaaatggaaaatgtacaacctttacaaacttacattttaaaaaatggtaatatagttattgttatgggtaatacatcaagttatgaattaacattatttgtacttgaaaaggatgattttgaacaattagaccataattcacctaaaaaaatcgttgatgaaataaggaatgaaatcaatgatttaaggagtgataataatggttaatgttgataacgcacctgaagaaaaagggcaagcctatactgaaatgttgcaactattcaataagttgattcaatggaatccagcttatactttcaacaatgctattaatttagtatcagcttgccaacaactattattaaactataatagttctgttgttaaattcttaaatgatgaactaaacaacgaaactaaaccagaatctattttatcttacattgctggtgatgacccaatagaacaatggaatatgcacaaaggattttatgaaacgtataacgtttacgttttttagaaaggagtgatataataatgaaacctgatgatattgttacattacgaataaaaggttatattcttcattatttagatgataaaaatgaatatgttgaagaatttatcccacttcacgagtatcatttatcaaaatcacaagctaaagacttattacctaatacacacaaacttttatccactacacgacatactaaaactatgcaagtttattacaatgatttactacaaattgcaattgcagaaagcaaataatttaaataagaggagaaataaaaatgacaaacgtaaaagatattttatcaagacaccaaaacacattagcgagattcgaatttgaggaaaaagaaagagaatttatcaaactatcagaattagtagaaaaatacggtatgaaaaaagagtatatcgttagagcattattcacaaacaaagaatcaaaattcggtgaacaaggtgttatcgtcactgacgactataacgtaaacttaccgaatcacttaactgaattaattaaagacatgcgtcaagacgaggacgttgttaacattatcaatgctggtgaagttcaattcacaatttatgagtatgaaaataaaaaaggtcaaaaaggttattcaatcaactttggtcaagtatcattttaatacaatttcataggggatatttatcccctatttttatgaggtgctaaacaatggaaaaaatatacactgccgtattattatacaatgtatcaattaatgaaacatatgaacatgaaattgaacaattcgaaaaaataaataaagctaaggtaatatatagttattttgatgcaaacttttataaaaaaggtgcttataatctttgtgtaaaatacattaaggagatataaaaatgaatattacaacaatattaaacacaaaaaaattaattaattatattttagataatagagattgttttatgaataaaataacaaaatttgaatcactaagtggaaaatgtgttgtttttgttagatacggtgaaatttctattgaatactatgatagtgatacaaaatacaatgatttatttactttagacattgacgttgatgttcgtacatatagttttagttgtcttgaggttttttatcgagaacatttaaacccattatataaaaaagaagtttttatggattgtactattgatgacgtattagaatatttcgagaaaccaatagaaagtaatataacaataatttttcaaaacaaagtgatatataataacgggaacgtgatagaacatgactaatatattaaacatagctattgttttccttttagcatttttaattacacttgtaatacttatgacaatgcatatacgtgtgtcatttggtgttttatttactacattgattatattctacattatctttttagtggttatatatgggttatatggaggtcgttagcaatggttagacatacgtccgaaatggataaatggaaaaaagaaagagacgcaagaaaagagcaggaaaaagaattgtttttaaatgattttagtactgttaattttaaatttgatgataaagatttacaagaggcgtacatagacgcatggaaacatttcgcacatttgccctattttccaaaagaaagaaacgtgtcatatgtaaatgctgtatcattggtaagaggtaaaagacatgaacaattaaactatatacttgaaatatataaccgtaaagatgaatctaataataaaaacgctaaaaaacataaatatgctttatatgaattacaagctaaaaataataattcttctatgtataaatatataaaggaaattgacactttatacaaagaaataggtaaatcagacagaccagtgactactattgatgatgaagatgtgagatataactttttatattatgcaacatttgaagactaattttaatactgtaaacgacataataaactattacaaggagcaaaaacatggtgaaacaaaatcgtttagacatggtaagagattatcaaaatgcggtcaatcatgtaagaaaaaaaataccagaaaactataatcaaatagaattagttgatgaactcatgaatgatgatatagactattatatatctatttcaaaccgttctgacggaaaatcgttcaactatgtttcattctttatttatttagctataaaacttgatattaaatttactttattatcacgtcattatacattacgtgacgcttatcgtgattttattgaggaaatcatagacaaaaacccactattcaaatctaagcgtgtcacattcagaagcgctagggactatttagctatcatctatcaagataaggaaatcggtgtgattacagatttgaatagtgcaactgatttaaaatatcattctaactttttaaaacactaccctatcattatatatgatgaatttttagcacttgaagatgactatttaattgacgagtgggataagttaaaaacaatttatgaatcaatcgaccgtaaccatggtaatgttgattatatcggcttccctaaaatgtttttattaggtaatgcggtcaacttttcaagtcctatattatccaatttaaatatatataacttattacaaaaacataaaatgaacacatcacgactttacaaaaacatttttttagaaatgcgaagaaacgattacgtaaatgaaaagcgtaacacacgagcattcaattcaaacgatgacgctatgacaactggcgagtttgaatttaacgaatataatttggcagatgataatttaagaaatcatatcaatcaaaacggtgattttttctatattaaaactgacgataaatatataaaaattatgtataatgttgatacatttaatgctaatattattgttataccttatacaaaacaatatgaattttgtactaaaatcaaagatatagatgacaatgttatttatttaagagaagatatgttttataaagaaaacatggagcgttattattacaatccaagcaatttacattttgataacgcttactcaaaaaattacgtggttgataatgatagatatttatatttagatatgaataaaattataaaatttcatataaaaaatgaaatgaagaaaaacatcaacgaatttgaaagaaaagaaaaaatatatgaagataactatattgaaaatacaaagaagtatttaatgaaacaatacggcttataaaaggtgtgtaagattatgggattactagaatgcatgcaatatcataaacatgaacgtcgaatgattttatactgggatatagaaacattagcgtacaataaagttaacggacgaaaaaaaccaaccaaatataaaaacgtaacgtattcagtagcaattggttggtttaatggttatgagattgatgtagaagtatttcctagttttgaatcgttttatgacgcattttatacgtatgtgaaacgacgtgatacaatcactaagtcaaaaacagatattatcatgattgcacataactgtaataagtatgacaaccattttttacttaaagataccatgcgttattttgataatatcacacgtgaaaatatatatttaaaatctgcagaagaaaacgaacacacactaaagatgcaagaggctactattttagctaaaaatcaaaatgtgattttagaaaaacgtgtgaaatcgtctattaatttagacttaacaatgtttttaaatggatttaaatttaatattattgataactttatgaaaacaaatacatcaattgcaacattaggtaaaaagttgcttgacggtggttatttaacagacgaccaacttaaaacagattttaattacacgatatttgataaagataacgatatgtcagatagtgaagcctatgactatgctgttaagtgttttgctaaactcacacctgaacaacttacatacattcataatgacgtgattatattaggtatgtgccatattcattatagtgatatatttccaaattttgactataacaaattaacattttcattgaatattatggaatcgtatttaaataatcaaatgacgcgttttcagttactcaatcaatatcaagatattaaaatatcatatacacattaccatttccatgatatgaatttttatgactatatcaaatcattttatcgtggtggtttaaatatgtataacactaaatacataaacaaacttattgatgagccttgtttttctattgatatcaattccagttatccttatgtgatgtatcatgagaaaattccgacatggttatacttttatgaacattattcagaacctacattaatccctacttttttagatgatgacaattatttttcattatataagattgataaagatgtatttaacaatgatttattaattaaaatcaaatcacgtgtattacgtcaaatgattgtaaaatactataataatgataatgattacgttaatattaatacaaatacattaagaatgattcaagacattacgggtattgattgtacgcatatacgtgttaattcgtttgtgatatatgaatgtgaatactttcatgcacgtgatattatatttcaaaactattttattaaaacacaaggtaagttaaaaaacaaaatcaacatgacatcaccttacgactatcatattactgatgacatcaacgaacacccttactcaaatgaggaggttatgttgtctaaagttgtacttaacggattatatggcatacctgcattacgttcacattttaacttattccgtttagatgataacaatgaactatacaatattattaacggttacaaaaacacagaacgtaatattttattttctacatttgtaacatcacgttcattgtataacttattagtaccattccaatacttaacggaaagtgaaattgacgacaattttatttattgtgatactgatagtttgtatatgaaatccgttgttaaacccttattgaaccccagtttattcgacccgatagccttaggtaaatgggatattgaaaacgaacagatagataagatgtttgtactgaatcataaaaaatatgcatatgaagtgaatggaaagattaaaattgcttctgctggtatacctaaaagcgcctttgatacaagcgtcgattttgaaacctttgtacgtgaacaattctttgacggtgcgattatagaaaacaataaaagtatctataatgaacagggtacaatatcgatttatccgtctaaaacagaaattgtatgtggtaatgtatatgatgaatatttttctgatgaacttaatatgaaacgtgaatttatattaaaagacgctagagaaaattttgaccatagtcaatttgatgatattctttatattgaaagtgacattggttcattttcacttaacgacttatttcctgttgaacgttcagttcataacaaatctgatttgcatatattaaaacgcgaacatgatgaattaaaaaaaggcaactgttaaataacagttgcctttttcttttgagataacatgaaaaatgtgtacgaaaattgattatgttttgtattttatttactagcattactagcatgtgttcattatagcatagttaattaagcaataccactgaagaatacaatattatcaccagcgttatgaggtacaccattaatcaatgtatataatacaacccgtgacggtgcgacgtatggtggtacgttatagtttgctactaagaatgaaccatcgtcaaatactgccactacaacacctgtgtggccaataccataagctgttgcttgtaagtatggtggtttacttgaaaaaccataaccaacagtagggttgtgtgttgttttagcacctaactttttatagacgtaccatacacgttgaccgtttgtcacttgaccgtcgtctgtcggttgtcttttaccatgtaattgtgacatatacgcccatgttaattctgtacactgacctgcattacccgtttgaggaaatatgttacctggtttgtataaatattcttttttgaataaaggtacaccaattgcttttttatatttttctggtaactgtgcgtatgtccagttaccaccaatgacgcgaccacttttaccattaggtttgactgatttaccactaattggtttatggtctccgtcatcatcagtagggtttgaactactacccccactatctacttgcacgctatcaatcagtttttttaatgaatcgagtagcccaatcgtcattttaatatgatatgtgttgttaaatgttttttgtaatgtaaaataatcattactaaaaaatttgtcgctacctatactgtgtacatcccattgtaatgcgtcttgtactttttttaataattcttgcatggcttgttttgctaaagcgagcagtgaactaccactgtcaccactactaccactgtcagacgaatcactaggtgaaccacctttaccgtctaatttaccaccccacgcaagaatagtattcgcaccgtctaaaaaaggattaccatagttttgtactttattatatgacgctttcaaacctaggggataatatgccgcccaagtagccgctgctgttaatggaatatacgcacgtccgattgtacctgctttcatatttttagcaaaatctgcattaccttttctttgtacgtcttgcggtacaaaatgcactggattaccgtaatcataccaagacggttgtcctgcttgttttgattgtgatactaattttcttgcaatgaatttagcgtctgttaaatagtcacctcgtgcagatgtatggttcaaccaacctaaaccagcgctatacccttcatttttttcatatacagcaaaaagagtaggtgacacacctatctcttttactgcttttaatacttgtctgattttactttcattaccacctagccatacattaaagcgtccatacccttttactttagggactaactggtctatcgttaaaccgaaatcatcattaatatacgaatgcgtaaatttatctatcttctcttggtcgttcattattatcactcttttcagaatcatttttaattactcttaatttatctttaatttgttctgggactaacacgtccatttctgcacaattttctacgatagataagccctcattagcgatataatagaaaatcgtaatcatgagtaagccaccttttaattgtaaaatctggtcaatgatgttagctaaaatgataatacagaatatcaataattttttagcaaaacctttcattgattttttagaccatagattattatttttaatagctttagcaaaaccagtaacaacatctataatcatcaaaacaaataaaaaatatagtaactttaaatcacctgcatatatgaacatatgaaatgcttctgtgtccgtaaatcttaattttacctcattcattttatacaccttctctaaatttattatttaacgggttctgtaacattggattacctgaaccgtcattatgccaaaatctcacaccagattctaaaattgcttttaattgttccattaacatagggtcaatgtcacgtatggtatacgtacctgtacattttaaatagttgcatacagtcatgctgttaataggttcaatgaatgtattatagtcatttacttcaaaaccaaacaacatataatatttttgtaaaaatgtaatttctttaggtgacggtacactaattttcatcgttaaaccgttaatgctatttgcaatttggaatgcgtttcccatttctgattctgtcacagatggtggttgtaaggctaaatctttatattctgcctgttgttgtttgtagaaattatattcttcattaaacttaccaaataaagcggttggacttaaattacttgctacacttacagcgtcataaaaacgtgattttgggtcactaccatttaatacattatctatacgacttgtgattaattgactttctgcattacgctgtctattggcttgttgtgattgtcctaaaattccgttattaattaatataggcacttgcgcaaaactattaaatgttatattggtatttaagaatgaacctgtatcaattaatatatctttatttttagcaagtatcggtctatcgttttcagcactgttataatcaactggatatactcgcacttcattatgataaccaatgatggattttgtacgtaacttaacacctgttttttgtgaaattttaccagcgtcaagtaacattgtgttaccgttccagtcataaaattcaatcgtcatgtactcattacgtatcatatgtttaaactcgtcttttttagacaacatcatctcttgaagctttgtgaaacttaatgataaatcgtttaaactccattcttttgattttccaccctgttttaatgtctttaatccagtaattttttcacttgtcttaacgtcctctaaatcttttgtattaataaaatctttaggtaacatttgaaccttttgaaagttttgtgtaatccatggatatgcactcattttatccataaagttaataaagtcaccatattccataacgtataagttgactggcgatgtgatattgtcatatatcgtacctttagacgtatctaagtttggctcttttttcgtaccaaatttctttgataaatcagcgcttgactggaataacactaaattttccaaatactgttgcatttggttatacacatagtttttatttgatacttttaacacatcatcattgttacgtaacatcggcaacatatagttatacgtacgctttgataagtgttgacgttcaatattaacgtttgaaagttgttctaatacattaccttgtgtatacgtcataatagtatcaatcacaaaatatatttttaccacaacatcattcacatattcgatttgattcacaaaagcgtaatagcgtctgtcctcaaaatctgataaaaacgtcatgtagttaatcccttgtgcgtcatgccactgcatatcaacattgatttccattctatcacgtataaaattatacggttgtttggaatagtctaatgatttaaaatgacgaccgtttaaaaaataatcatcacgctctttattactattaaaatgaattgtattttgatagtctgtaaacggtgtgttatagaaaaatttaaaatttgttaattttctcatttttacctccataaaaaatagtcgtataaataatttatacgactattataacatttttattcaatgatttgtgtatctattgcaaaacgtttatctccatttgttaagtcactatcgctataatttgatgtaacaaaatgtaattcattattaaagtttaaatataatcttgtattaatcattttcgaatcaatcgcacattgtgtgtagtgatgtgttgattttaagttagtgttaatcgtacctaatttaatatcaccgtttttcttaatcccttttaataccccttttaattgtatggttttaacaccattaattgttacaatacgatattgcggtgcgggataaccaccgttgctatcacttgcaactataccactttctagcaatatatcttgccaacctgtatcattaaattgtaattttgcactgttgattttttgatcaaaattttgttcaatttctgatttagattgtgcaattttagtatctatatttaagttgtccactcttgttgataaagtgttaatacttgattcatttgttgatgttttttgttttaaatcattaatatcattatcatattttgataaatcaatatgtccgtttccgtcaacaactggtctatcttttaattcttgaatatctttttcaatttttgatattttatcattatttgtttttaaagcagcgatgtcagatttaattgaattaatatcctgtgtataatctttttggttaatactgtttaattttgtttctaactcttttatcttatcaaggttggcatttgctttgatgtcattaatggtttgttctaaactttcaagtttcccagcacttgaattttgttttaacttattaatatcctcttgtatttcttgaattgtgttgtcattgttttgtttagattttaataattcaatctgttgttcaatatcttttattttatcaagattgatactttttttaacttcagctaattctctttttaactcgtcaatgttttctgcaccagtattagctttaattaaattaatttcatttgtgtttcgtcctacactatctttagtagcgtttaacgattgtgtaagtgtttcatatttatcatcatactgtaacattgatttttcaaattcatttaattgtgtttgatgttgttctaaactttcatttaatatgacatctttttcttttaatgaatcaatctcttttttattactatcaatcattggttggaatttttttattttctctagaaatgttgatattttctctttattatcattaattgcaatatcatgattttctatttgaccactgtatttattaatcaatgtttttaaatcattataaaatgttaatttataataaccagtatcagtacggacataaatatgtccgtctgatgtactgattaagtcattttcttctgttaaaaaatctgctaacctgtctattgtttcaacttgtcttaaacttcttacgattctatcagccattgttcacacctcttatttgtatcgtttccaactaaactcaaagaaaaaacctaaaatacccattatgagaacaccccccacggaataccaacactgtaactattacctgttttaccgttccattgtctcactggtaaataataacgtgtaccttgccaattgtaaccaatccatacatacccgtctgataaacaaacttcgtcatatggtgtatagccgtttggttggaaccaatagccattaagctcagataatttaggactaccgacacgagcaaatattggtaaaaaaccacatgtaaatgtagcattttcatttctgtaatatgtgccgtattggttttgtttccagttgttagtttgttgaatatttttttctaatactttactatcactatttgaaaattttggacgaataaagtgtgttacaccgtcataataatgtgttcttattgttgctttttcccaaccatcatatccaccatttaaccagttttgttctaagcatgtataataatcaagatttccacttgttacacattgaatatgcccatattgagaattagtgtatacagcaacgtcacctaattgaggtttaaagctaggtgtattttcatacaccgctgctaaacctttaaagtcgttgttaatggcgtctttggcattaccccacatgcgaactttaccgtcagtaatgtaataaacataagcaactgctaagtccatacattgaaaaccatatgctccatcaaagtcaacgccaacaccctcatgtttatatatccaatctttagcttgttgttgtgatttcattgcacattatcctttcttaacccaatcatactcacgatgtttcgtttccgcattaacgttactaaagaattctttatgctctttaacattagcctctaatgtttgtctgacttcaccaacggcgttaccacttgaaacacgtaaccatgcacccacttttttaatttcttctggagcgtctctaaataattccatttggttacctgtaatataatattcaccaggtattgtaacgtaagctatccagttattatattgacttgcttctaaatactcttgatatggtgcgtctgttttgattgttgtccataaaccataatcccattttaatgtaaatacatcgagcgtcataccacgcatgattttaaccatttttcgaccagtagaaaaacgcgttaattcttgtactgtacctaatgtttgtgttgtagggtatacattgatgaaacaaccagcgtctataatttttttactaccattcattggcatatttttaagtttactagctgtaccaccgtcaatataatagaaccctgcttgtgttaagtcatttaagtcgtcaatatggtctggaattgataacgcacgtccatcgtcttttgttaatttatagttttgtgaacctctcgcacgtaatgcttcaaagtgttcatattctccaagttggaagaaaccatataaattattgaatcgttttccaccaccgccatttgtcatagcaagtaataacgatttacgttttgtttttggattagtataaatacaaataccctcaggctctttaaagttatcacgtgggaaattaattccgtcttgatatgataatttaaacggataatcataaatcttttgacctgttgttaatgaatatttaccaatttgaacgtgtgaattaactgaactgttaccacttaaccagtataaatcatcaccgtcaacagcaatcccttgcatccatcgattatcattgttttctgaattgtcaatcgtcatttctttttctacattatcaatgtgattttttacgtcagctcttgaacgtacctgtattgtcccgtcgccgaatcttaaaacgattttgtcatttgcttcatcaattaacggtgtaaacgtgtgtttgtttaaaagtgaatgtggtgtataatctgttaagccttttgcttcttctaaatctaaaatataattatctttatatgcaacttgtaacagttttgctacaccgtcatgatgtaaccatattttcatttcaccattggattgacgttctaaacctattgtagtaccgtgaccaccttgtacaatacgcatactagatattaaatcaccactaggcgttaatttattaatccaaaaaccctctggtttttgtgaatctgattgtgtagagtacatatggtttgtttctttatcaatattaatagattggttaacagcattacgaataccaccaaaacccatcacaaactttggttcaagttcatttaattcaaaaccattaacgaaacggtcaatatctttgattaagtctttcacttctgctttaaagtcattcatttgtttcatttctgcaactttaaataatgcaaatgcggatgtaagaccagcactatatttagtaaattcatcatgaatgatgttatctattgtaccgtcattgagccaacctctaaataagtctttagcctgttctgggaatgctttcattaaatcgtcccagtttctaaaacgtttttttaactcaccgtcatagtcccaaatacgatgtgctaatacttcaatgagctttgataatcttgaaatataatcataatatgattttgaattggtattataatctgctctatcatcgtaaaacggtgtgtaacgttctctcgttttatatatttcgtctaaaaatggacgaatgtcgtcaaaatatttaaaatcgttttcattatatgccataattttccacctttaccaaatttgtaaaaaacattttttatcaaattcatttaaaattttctttcttaaatcgtatactttatcaatattatcaattaaatactgttttgaaaattgtgtgcctttcgcattacctttttgattttgattacgttttgcgttttgattactttcgttacttgatttattcacagttttaccgttatcaattgtattattatctgcaaatcgtaacgttgtattatctacatctatgttaacctcgctttgtggtaatgacacataagcatttctgtttgctgtcataccagttgaattgtctaatgatgttgcattttgatttgaggtttcatcagtgttgtttgttgtatcttcattatgttctgtaaaaccttgtgattgtagatatttttcaacttcgcttgatgaataaacaacattcaaataatcctcatgtgtgatacatacagtaatcacttgcataccaaaagcttcaactgtttgtctgttgatttctctatctaaaaagtgtattgtaaatgattttttaaaaagtaagtcagataagtcatctttaagtttaaaacctttaaacactttttcattaacgatagctaaaacgtctttgtcaaacttcaacattttttgcatgaattgaaattcatcatcataaaacgttaatttatcattatttacaaattcattaaaaccttttttaattaattctgatttaataaaatcaaataaagtcattgtatatctagccattgtattcactactttcatcattagacaatgattctatcattgtaatttctgaagtaacttcatcgtcgtaatacggtttaatatctaagccataacgttttgataagaatgtaatcggctcacgaccttttaaataaatattactgtttgatgttgtaaaacctcggttactttttgcctcttcgtcagaaacaccgctttccttatccactgctagtgaattaatacctaaatagttacttaattcactaattttattttggtattctcttttcatctcagttagtgctggaatcacactattactggttaaatctatgatatcatcatcggcgttaaacataggtgacattttaacaaatggtgcaccgttgtatatttccgatacaagttgattaactgactcatcatttatgtctgatttaaatattttgctaaatttcgcttgcataattaatgaaaaacgagataaaacaacctcagataattcatcagtataatgttctataatttctatatcactattatattgaatgggtttattttgcataacaacaaaattaccactcatacagttatcatatattttatgaatctgtaagcattcatctggtattaaatagtctggaacaatgaaataaatatcatcttttgttaatcgtttttgaaattgaaaattgaaatttgatgaaaagtttggggcttgattaaaataagtgttgttaacatagcccaaaatcataatttgttcatttcttgctttaccaacaacaacattaatgttttgccttaatgcagattctaattgaataaaatctataccaaccgtatcacgattggtatagtttattaataggggtaaaaattccaaataacgattaaacataagacgtttaaatctgttgcgatgttctacaacacgtttattgatttctttagataattcaacctgtacgcctttattatgtttagtcatttatatcacctctattattctgttctcggtgttacatcttggtcagtaattaaaattttgttaaagaatgggctaatggctttaaatgaatagtaatgaatccagtgtgtaacttcatcaaattcaccattatagaatggttgttttaacatgccttttgtgtaacgtttatatttaattgaattaatatccaaaataaaagcgtataaatctgattttggtttaatttcttcaacactatctttaaattcagatagttttgatacatcgtaagtgaatactgaaccaactggaattgtatcacctttatgagtttgataatcaccgtaagcacgtaagaaattaactgattcatcacttgatacgacaatatctttagtgactttaaaaacaccacctaaatcgtcgaaactaataacatggtctgtaaagtcaatccctgctacttggaaagtgttcgcaatctttgtatctaaaagataagattttaagctatcagttgttaaaataacaatatcttttaattttgaaactgttgtatattgtccgattgcaccacctgaagcacgatgaacttcattatatttagtgctgttgttttgtaagttaagaatagcttcaaaaactttacttgctaaatcttcttttgatgttgctttacgtacatttgattcagataattgattcaatgagtaatcaactaacattgcacgcatttctttttcttctaatacgttaatatcagaaattttctttttatagacacctaatgcataattagttgcgtctgctaatgtttggaaattgaaacgtgtgtcattgttatttaatgtgaatttttgtttcttcacaataccactaccatataacttagtagccatacgtgggtaattacgtttcaacattaattcttcatttttagataagtccatgttaataggtactgtatccataattacatactcttcactgtattgacctataaagtcttgctctttagctaaccaattaaaacggttacctaatgcaatatcgattaataacgtttcattaatcttagggaataaaaatttatttacaaatgtttcaaacattgtattagaattatcccacttatcgccaaacgtccatgattttgaataagtatgattaaagtcttgtaatgcagatttagctgactttgcaactaatagtgctgtttcgttttttgtactcttttctgccatgatttattattcctcctctacatcgccagtaaatgactgttttgaaagtgaatgaacttgtacaccataactatcttcacttttgtttgtatcaattgacatattttcatttaattctgttcgtttatttaatcttgaatcttcatatgatgttcccatcatagaacgcatattgttaccctcatacatgtttaaattcctcctaatctaaatctaacttatccactaattcttcatctgaatagtctttgtcgttgtcatctgcttcagtaacatctggttttggttgtggttgtggttgtggttgttgcatttgtgatgtttcaaaattagtaaatcgttgttctaatgaagcgatacgttgttctaaaacaacagggtctaattttgcactatcttcatcagtaatagtaggttctaatttgttttcattttcttcttcgattgtttctacttttttatcttcagtagattcttcagttgattcttcagttgattcttcggttgattctgaagtttcttcagttgattctgaagtttcttctttgtcgtctggttttacgatttcatcaaattctgtcattttgacacctccaaatattttataactaattatatcatagaatatttaaataagtaaattaaaattattttaagttcaaatcataacttttgataaaagtcaatagatacataaattttgtatttgatgaatatgttataggttagataagttggaaaagtagacgtacagtgtttataagtttagtaaagaaatgataagta

SEQ ID NO: 46 - DNA, 140909 bp, Staphylococcus phage sp.
  source 1..140909: mol_type = genomic DNA; note = SaGU1
atggatggaaaagaactaattaagatagcacaagaaacatttcaaactgaaaaaataacaagagaacagatagaccatataatcaatatgttaaacccttctacctatatgcttaagtatcacacactaagaggacaccctataactttcagcattcctaatagagatagaagtaaagcacaggctcatagaccatggcaaactaggattgtaaacgatactcatcctaataaggctgtaataaaatcacgtcagttaggtcttagtgagatgggtgtaatggaaatggttcattttgcagatatgcatagttatgccaatgcaaagtgtttatacacattccctagattataagttggggaattaaaactctgttaaacggtcataggaaaataaagaatatcctggtaagagagactaagtcctaatggatagagttaataccgtgctaaatcaacctaataagttgtaaatgccgaacgactaaatttctagatagcttattaaaagggataagtgaaaactagataagaaagtcttgacaaaaataccaaagtaagttaatatattaaataggaggtggaaaacaaatgcctaggaaaaaaacacatgaagaatttgtaaaacaagtaaaagatatagcaggaaatgaatacactgtaacaagacattatgaacacgcaaatcaaaatataaaaatgtttcataatgaatgtaaggaagaatttgaaataagaccaaataatttcttacaaggtaaaagatgttctgcttgtagaggtaaaagaataagtaagacacaagctaaaccacttagtaaagttaaaagacaattaaaagagaaacataatggtactatagagattataggtgagtatacaaatactcacactaaaacagattttaaatgtttaacatgcaatcatactttctcttcagaacctaattctgttttaaggctgagtggatgtcctaaatgtaaagtttctaaaggggaaaaattaatagctgaattccttaaagaaaataatatacgttttaaagaacagaaaagatttaaagattgtgttcataaaagacctctaatatttgatttctacctattagatttaaatactttaatagaatatgatggattacagcacagtaagccaatagattattttggtggtgttaaagcttttgaagaattgaagtatagagatagcttaaaagatgattacgcaaaaagaaataatattaggatggtaagagtgtcctataaacaaaaagagaaagagataatagaagaattagaaagtgttttgggtgtggtaacccgtcaagaaagtaaagcagagagctcaacaccaaaaagttgagttaagatatagtctagtccgtatgtaaatatcacgaaagtgacggtagtaacgactaatgaacaaatgaaaaaatttgttcagtctcgtttgaaccctgtattagaaaaagaatattttagagatattgttgattgggataaggactctttaggttttaaaaagataagaaactctagtttattctttagaacaagttctaaagcaagtactgtagagggtgtggatattgactacttatccttagatgagtatgatagggtaaacttattagcagagtcatctgcattagagtcaatgtcttcatcaccttttaagattgtgagaagatggagcacaccatctgtacccggtatgggtatacacaaattatatcaacaatctgaccaatggtattatggtcatagatgtcaacattgtgattatttaaatgaaatgagttataatgattacaaccctgataatcttgaagaaagtgggaatatgttatgtgttaaccctgaaggtgtagatgaacaggctaaaacagtacagaatggtagttaccaatttgtttgtcaaaaatgcggtaaaccactagatagatggtataatggtgagtggcattgtaagtatcctgagcgtacaaaaggtaataaaggggtacgaggatacctaataacacaaatgaacgctgtatggatttctgctgatgaattaaaagaaaaagaaatgaatacagaatctaagcaagcgttttacaattatattttgggttatccatttgaagatgttaaactgagagttaatgaagaagacgtttatggtaacaaatcacctattgcagaaacacaattaatgaaacgagatagatattctcatatagctattggtatagattggggaaatactcattggataactgttcatggtatgttacctaatggtaaggtagacttaatacgattattctctgttaaaaagatgacaagacctgatttagttgaagcagatttagaaaaaatcatttgggaaatatctaagtacgaccctgatattataattgcagataacggagattcaggaaacaatgttctaaaactcattaatcattttggaaaagataaagtatttggatgtacgtataaatcttctcctaagtctacaggacagttgagacctgaatttaatgagaacaataatagggttacagtagataaattaatgcagaataaaagatatatacaagcacttaagacaaaggatataagtgtttatagtacagtagatgatgatttaaaaactttcttaaaacattggcaaaatgttgttattatggatgaagaagatgaaaaaactggagaaatgtatcaagttattaaacgtaaaggtgacgaccattatgcacaagcaagtgtttatgcctatataggattaacaagaataaaagaacttcttaaagaaggaaatggtacaagctttggttctacatttgtttctactgattacaaccaagaaggaaataaacaattctactttgatgaatagaggtgaaatagacttgacagataaattattttatggtacaattagtaatgaagaaattaataaaagtgtattgaatttgttattaggtgaggaattatctttagattatgtttctaaaaatagtgataccttagatgttaaatatgaacatgtttataaatctctaggattcgataatttctttgattgttttttatatgctaatagagaacctgaaatagtccataaaggtggagataaaaatcttggtgggctaaataaggttaaacgtactgttattcgtaatggtaaagaaatggaaatgacagtttacgaagacggtaataaagaaaatgatagtaaagaaaaacaagaaggcaaagaagaagttagtagaagtgcagtaggagcaagagctatttctaatggtgaagaaggaaaggtaaaccctaaaaaggtagcaaattcattatctagtttaagtaaaaaaggtgtcgatgtatcacatgttagtacaaactcatcattgtacaaagagtttgttgatgataatggggatacattaggaattacatcttttaaaagaactgaaaatgatataatattagaatcttatgcaagttcacccgattcagatggtgtaggggctagagctattatggaactattacgtttaagtattaaggaaaataaaaatgcagttgtgtatgacatagaattgcctgaagctatagagtatttaaaaactttaggatttaaacctaataaagatggatacatcttaagaaaaaaagatgtaaaacaattcttaggtgattatagtgattttatttagcactgtagtcatctattctattgtatttattctctatattatattaaaaacaatttatgtaaagtctaatatgaatagaatagataacacaactgaattattaaaaatattacaggaagatattgaaggaaagataaaaaaggaaggaagaaataaatgactttagaagaaaataaattaacattagaagaatcaataactccacttagtaaagaggagaaagaggatagtattaaagaatttagtagtttattatgtgaaatggtaaatagattatacaagtcttacaatgtatttagacaagaccctatggatgaaacgcaacgtttagatggttctctaatggtctttcaaagtagactaaatgaccctttaacaggagatttacatgacaagatgtataaacttgctttttcaaaacgcattgatatttttgaagctaacaagcaattcagaaaagatgtagaatcaggtaaggcaattgaactaggtgatgtagctattatagatacagcattaagtaatattctttcaggtaacgagttccaaggaagtatttcgtttatgcttagaaaagactttgaagaaaaagaaagaattagaaaagaagaagaagagaaacttaataacttataaaagggaagaattatgagactatataaaatgaggtatcataattgaaaaagaaaccacaaggcaatgagataatcataaccataataacggttatgatagcaatatttgtagtcattatgaccatattttttaataaataccaagatgctaaagaagataaagatagatatcagagattagttgagatttataaaaaagcagatgataatgatggagagactaaaaagaaatacgtaaaaagattaaataaagctgaagaagaacttaaaaaagtaaagaaagaaacaaattataaagactataataagaagtcaaataaagaaagacaaaaggaagataaagaaactagagagaaaatatatgatgtaactggtgatgatgacttaatattagtaaaaaataatattgagtttagtgataaggtagataaacctgaaatacttattagtgaagatggaattggtacgataactgtccctacaaacagtggttatgaaaaacaaacagtaggttctattattactagtgtattaggttccccgttcttatcaactgattcacccggtatagatagtttaggtatcattaatagtaatgtttatccaaatacagtagatagtatagtagaagatacaaatacttctactgataatgtactaaaggataatcctattataacaaatccagttgaaccaaccacaccttcagatatattacctcctattgataatccgtcagttcctatattacctgaaaatcctgtagacaataattcaggaaatatagataatacggataatccaaaccctccacctccaggatatacagatgaagatggaggtagaggctcaggtggtggaggtaatgttgaacccccaccaacggaagaaccttcagataacggtaatacaggaggaggagattgggaagaaaaacctgacccaggagaagagccatcagataatggtaatacaggagacaatgaaggagaggtaactcctgaacctgaccctacaccttctgagcctgaacaacctaatgaaaaacctaatgagggtaatggtaatgaagaaaaaccatccgaaccatcagataatcctgatgaaaatggaggatgggaaactgagccttccgaacctgaaacaccttctgagccggacgataaggtggacgaagaggataaaaacgaagatacaacagaggataaacaacctacagaacaaccggacgataataatacagataatgaggataaaactgaagaggagtaattactcctcttttttgtttgctatattaaataagagataaatataaaaaaattgaacattacggtggtgaaaactttgataggaatgaatgttataacgtcactatcagtagtatttacctgtttaagtcttttaactttaatgatttttgttcacagtaagttctccagtaaaaacgtttttgttttgtatgtaatttatgctataataggaataggtacatacatagttttaactatgtttcaaacaacatctgtacttattaagaatgatgtaatagattccatagaaaatactgaacattatattggattcaatgaccctataattatatttactataagttttataggggcaatacttggaggaatttggtacaagatgatgaaaattattaaaaagagtaactttaaagataaaaaataaaaaagacggtgaataggttgatattctctaaagataagaaatgggatgaagcaaaagatttcatcaaaggtcaagggatgcaagataattggatagagattgtagattattatagacagataggtggaaaacacgtagctgtttttattgctttaaacaaagtaaaatacatgattctagaagcaacaaaagacaataaagtaatattagtagataaagataataatatactattagaagattatgatattgttatggaaagtaaaaaaatgttttattacattgaagaaccgttcgaggttaaaataaatatccctcgacatattagagatgtaacttataataatactgttgtattaactacagtaagagggagtagaggtgactagtaattggcagatttatttaagcaattcagattaggtaaggactatggtaataatagtaccattgctcaagttcctattgatgaaggattacaagctaacattaaaaaaatagaacaagacaataaagagtaccaagacttaactaagtctttatacggacaacaacaggcttatgcagagccatttatagaaatgatggatactaatcctgaatttagagataagagaagttacatgaagaatgaacataatttacacgatgttttgaaaaagtttggtaataatcctattcttaatgctatcatacttacacgttcaaatcaagtagctatgtattgtcaacctgcaagatactcagagaaaggtttaggttttgaggtaagattaagagacctagatgcagaaccaggtagaaaagaaaaagaagaaatgaaacgtatagaagattttattgttaatacaggtaaagataaagatgtagatagagattcgtttcaaactttctgtaagaaaattgttagagatacttatatatatgaccaagttaactttgaaaaagtatttaataagaacaataaaactagattagaaaaattcatagcagtagacccttctactattttttatgcaacagataaaaaaggtaaaattattaagggtggtaagaggtttgttcaagtagtagataaaagagtagtagctagttttacttctagagaattagctatgggtataagaaatcctagaactgaattatcttcttcagggtacggattatcagaagtagagatagctatgaaagaatttattgcttataataatactgaatcatttaacgatagatttttctcacatggtggtactacaagaggtattttacaaatacgttcagaccaacaacaatcacaacatgcattagagaacttcaagcgtgaatggaaatcaagtttatcgggtatcaatggttcatggcaaattccagtggtaatggcagacgatattaaatttgttaatatgacaccgactgctaatgatatgcaatttgagaaatggttaaattatcttattaatattatatctgctttgtatggtattgaccctgcagaaattggtttccctaatagaggaggagctacaggttctaaaggtggttctactttaaatgaggctgacccaggtaaaaaacaacaacaatctcaaaataaaggtttacaacctttacttagatttattgaagatttagttaatagacatattatatcagaatatggagataaatatacattccaattcgtaggtggagatactaagagtgctacggacaaacttaatattcttaaactagaaactcaaatatttaaaacagtcaatgaggctagagaagaacaaggtaagaaacctattgaaggtggagacattatcttagatgcttcattcttacaaggaacagcacaattacaacaagataaacaatataatgatggtaaacagaaagaacgtttacaaatgatgatgagtttactagaaggagataatgacgattcagaagaaagtcaatcaacagattctagtaatgctgataaagaaataggaacagatgcgcaaataaaaggtgacgataatgtttaccgaactcaaacatctaataaaggtcaagggagaaaaggagaaaaatcttctgactttaaacattaattaataagcctagaataaatctaggctttgtttatttttttgtaatttaattttgataaagggaataactatgatatactatatgtaattgatattaatacataaaaaatattaatatttcacttacaagttattattgttatattattaacgtaaaagtaaataaaataacaagtggaggtgtagacacctttggaagaaataaaatttaatgcttttgtacctatggatttgaagaaatctgtatcaacagcttctgatactaatgagtattctatagtttcaggatgggctagtactccaagtatggatttacagaatgatatagttaatcctaaaggaatagatatagagtattttaagtcacaagggtacattaattatgagcatcaaagtgataaagttgtaggtatacctacagagaattgctatgtggatatagaaaaaggtttatttattgaagcaaagctatggaagaatgatgaaaatgttgttaagatgcttgatttagctgagaaattagaaaaatcaggtagtggaagacgtttaggtttttctattgaaggtgcagttaaaaaacgtaacataaatgacaatagagttattgatgaagttatgataacaggagttgcattagttaaaaatcctgctaatcctgaagcaacatgggaaagctttatgaaatcatttttaactggtcacggtacatcacctgacactcaagttgatgcaggagctttaaggaaagaagaaatagcatctagcattacaaatttagcttacgtcactaagattaaagatttaaaagagtttaatgatgtatggaatggcgttgttgaagatttgagtaaatctaatagtatgggatatgaggaatcagtccttacgttacaactagctaaaggtttatctcgtaaagatgcagaactagcagtaatggatataaacaaacaaaaactagaataggtaaggagaatacattctatgagtaaagaaatgcaaaatattttagaagagtatgataagttaaatgctcaagaggcagtttcgaaatctgtagaagatgatgaaaagaatacagtagaatctaccgaagaacaagtagcagaaacaactgaagaaccggctaaagaacctgaaaaggttactgaggaagatgctaaagaagcacaagagcaaggtgaaaaagttgaatctgaagaggtaacagaggacactgaagatgaggaagttgaaaaatcagctaaagaatcaaaagaccctgtagaccaaaaagatactaaaacagaaaataaagacaacgagaaacgtaaaaataaaaaagataaaaaagaagattctgaatctgatgatgaagacaaagatactgacgatgataaagataagaaagaagataagaaggaaaaaacttctaaatcaatttctgatgaggatatcacaacagtatttaaatctatcctaacatcttttgaaaacttaaataaggagaaagaaaactttgctactaaagacgatttaagtgaagttagtaaatctattaatgagttatcagcaaaaatttctgaaatccaatctgaagatgtttctaaatcagtagacactgatgaagaagaagctgtagaaaaatcagtaacatctacaaatggggagcaagaaaaagtagaaggttatgtttctaaatcagtagacactgaagagcaagctgaaactggtgaagcaaaatcagaagatgctgaagaagtacaagaggataacacatttaaaggattaagtcaagaagaaagaactaagttcatggattcttataaagcacaagctaaagaccctagagcttctaaacatgacttacaatcagcttatcaatcttacttgaacattaacactgaccctactaatgcatcagagaaagatattaaaactgtaaaagactttgcacaaatttaattaatgcacaaagttgtgttatattatacggtgtaactaaagaatataaatagggtacattttactgtaccctacataaaataaaaagaacacaaatgaaaggtgataaatttatatgactatcgaaaagaacctgtcagacgttcaacaaaagtacgctgaccaattccaagaagacgtagtaaagtcattccaaactggttatggaatcactcctgatacacaaattgacgcaggagctttacgtagagaaattttagatgaccaaatcacaatgttaacatggactaatgaagatttaatcttctatcgtgatatctcacgccgtcctgctcaatctacagtagtaaaatacgaccaatatttacgtcacggtaacgtaggtcactctcgtttcgttaaagaaatcggagtagcaccagtatctgacccaaatatccgtcaaaaaaccgtatcaatgaaatacgtttctgatactaaaaacatgtcaattgcatcaggtttagtaaataacattgctgacccatcacaaatccttacagaagatgctatcgcagttgttgcaaaaacaattgagtgggcttcattctacggtgatgcttcattaacttctgaagttgaaggtgaaggtttagaatttgatggtttagctaaattgattgacaaaaataacgtaattaacgctaaaggtaaccaattaactgagaaacacttaaacgaggcggctgtacgtatcggtaaaggtttcggtacagctacagatgcttacatgcctattggtgtacacgcagacttcgttaactcaatcttaggtcgtcaaatgcaattaatgcaagacaacagcggtaacgttaacactggttacagtgtaaatggtttctactcatctcgtggattcattaaattacatggttctacagtaatggaaaatgaattaatcttagatgaatcattacaaccattaccaaacgctcctcaacctgctaaagttacagctacagttgaaactaagcaaaaaggtgcttttgaagatgaagaagaccgtgcaggattatcatataaagtagtagttaactcagatgacgctcaatcagctccttctgaagaagtaacagctacagtatctaacgttgacgatggtgttaaactttcaatcagtgttaacgctatgtaccaacaacaaccacaatttgtttctatctaccgtcaaggtaaagaaacaggtatgtacttcctaatcaaacgtgtaccagttaaagatgcacaagaagatggaacaatcgtattcgtagataagaacgaaacattgcctgaaacagcagacgtattcgttggtgaaatgtcaccacaagtagttcacttattcgaattacttccaatgatgaaattaccattagctcaaattaatgcttctattacatttgcagtattatggtatggtgcattagcattacgtgctcctaaaaaatgggctcgtattaaaaacgttcgttatatcgcagtttaatagaataagaaaaactgaatacaagagaatagggataaacttagggtttatcccttttttattaaaataaacttgaagggactcaataaatatgttatactataagaaattactaaatacaaaaatggctaccatttatggtacagtagatattgacaaagatggagtagttaaaggattaactaaggaacaagaaaaagagtttgcaaatgttccaggttttgaatttgaagaagaaaagaaaactactagaaaacaatcagcttctactagtaaagaagaagaacctaaagaagaggaaaagaaagcctctactagaaaaactacaagtactactagaaaatctacagcacgtaaaacaacagccaaaaaagatgaaaataagtaaagggtgaattaaatggttaactcaatgtttggaggggacttagacccttatgaaaaatcattaaactatgaatatccttatcatcctagtggtaatcctaaacatatagacgtaagtgagatagataacttaacattagctgattatggatggtcacctgatgcagttaaagcttatatgtttggtattgtagtacaaaaccctgatacaggacagcccatgggtgatgagttttataaccatatattagaaagagcagtaggtaaagctgagagagcgctagatatttctatactacctgatactcaacatgagatgagagattatcatgagacagagtttaatagttatatgtttgtacatgcttacagaaaacctatattacaggtagagaacttacagctacaatttaatggtagacctatatataaataccctgctaactggtggaaagtagagcatttagcaggtcatgttcaattgttccctacagcacttatgcaaacaggacaatcaatgtcatatgatgctgtattcaatggataccctcaattagcaggtgtatacccaccatcaggagcaacctttgcacctcaaatgatacgactagaatacgtatcaggtatgcttccacgtaaaaaagcaggtagaaataaaccttgggagatgcctcctgagttagaacagctagttataaaatatgcattgaaagaaatataccaagtatggggtaacttaatcattggtgccggtattgctaataaaacattagaagtagacggtatcacagaaacaataggaactactcaatcagctatgtatggtggagctagtgctcagatacttcaaataaatgaagatataaaagaactattggatggtttaagagcttactttggatataatatgataggattataaggagggttagaaaatggaaaaaccgtatatgataggagccaactctaaccctaatattattaataagtcaacaacatacactactacaacacaagcagatgaacaggataaacctaagtatactactaggctagagtttgatactattgatatgattaggtttattaatgataggggtataaaagtactatgggaagaagcatatttttgtccgtgtcttaatcctgacacaggtcatcctagagtagattgtcctagatgtcatggtaaaggtattgcttatttacctcctaaagagactataatggcaatacagtcccaagagaaaggaactaaccaattagatatagggatattagatacaggtactgcaataggtacaactcaattagaaaagagaatatcctatagagataggtttactgttcctgaagtattaatgcctcaacaaatgatttattttgtaaataaagatagaattaaaaaaggtataccattatactacgatgtaaaagaagtaacttatatagccactcaagatggtacagtctatgaagaagattatgaaattaagaataatagattgtatttaaatgaaaaatatgagaaccatacagtaactttaaagatacttatgactttaagatatgtagtatcagatatactaaaggaaagtcgttatcaatatactaaatttaatcaacctaaatcaaaatttgaaaacttacctcaaaaattacttcttaaaagggaagatgttattgtactacaagacccttataaagttaatgatggtatagaagaagacctagaaattcaagtagatgaccctaaagcttccgcatctaatcctagtaatttaggtggattcttcggaggtgcatttaaataatgccagttcacggaaagagacctaatttatttaaaaataaaaactataagcaggtaggtaagagaaccattgatggcatgcgttcagaagttcttgacaagctacaagcaacagcacagcaagtagagaatactagtattaaacgtatgcctacttatctacaagtaacagagaaaaagcttgaaaaagaaggagtagtagaccttaaaaaagcatttgctcactcatctaaaaagaaaactagtaaagacggaggatggtatttaactgtaccaatccgcattaaaactagtagaatgaacaacagtacttatcaagatatgaggactttaaaagtagataaaggcacaggttcagtttctaagataactgattatctagaaggacgtagaaagaatgtaagtcacccgtcaatgaaaccggaacctatgactcataacatgactaaagttaaaagaggaaagcaatcttcttactttatatttagaactgtttctagtaaatcacctgctagttcctggatacttaatagagataaagttaatgagaataatttctctaaaacaactctaaaaactgttaagcaactaatgaactggaagatgaaaaatttaaattaagaggagggttagtattaaatggcaataacatcagttgattcatatttattatcagaaataaagcctagacttaacactgtgctagagaattgttatattatagatgaagttttaaaagactttgattatcaaactagagagagctttaaagaagctttctgcggtaagaatgcacaacatgaagtaacggtaggatttaacttcccaaaatttaaaaataactatgaagctcattacttgatacaattaggtcaaggacaagagacaaaaaactctttagggagtattcagtcatcttactttgaggcaacaggagataccttagtcgaatcttctacagcaataagagaagatgataagttagtttttactgtttctaaacctataggagaattaataaaagtagaagatatagagtttgctaaatacgataatcttcaagttgaaggtaataaggtatcatttaagtaccaaacaaatgaagattatgagaattataatgctaatattatctttactgaaaagaaaaacgactctaaaggtttagtaaaaggattcacagttgaagaacaagtaacagttgtaggtctatcatttaatgtagatgttgcacgatgtttagatgctgtattaaaaatgattttaatatctatgagagatagtatagaagagcaacaaacattccaattacagaatttgtcctttggtgatattgcaccaataatagaagacggtgactcaataatttttggtagaccgacaattattaagtacacaagttctctagacttggattatactattacacaagatattaataaactaacttttaaagaaagaaaggattggaagtaggatggctagaaaaaagacacctgaaaataacactcctaaatttaatggttatgttcatatagatacattccttgatactgcaaaaaccctttttaacatgagggattcacaagtagcaggatttaaagcttatatggaaggtagtcattatttgtttagtgagcaagaattcttaccatcattagagaagtatttaggtaggaaattagatatataataacattcagataaggagaattaaatatggcagtagaaccattcccaagaagacctattacccgtcctcatgcatctattgaagtagatacttcaggtatcggtggctcagcaggttcaagtgaaaaagtattttgcttaatcggtcaggctgaaggcggagaaccaaatacagtttatgaattacgtaactatgcacaagctaaacgtttattccgttcaggagaattacttgatgcaattgaattagcatggggttctaaccctaactatacagcaggacgtattttagctatgcgtatagaagatgctaaacctgcaacagcagaagttggcggactaaaaattacatctaaaatcttcggtaatgttgctaacaacattcaagtaggattggaaaagaacacattaagtgattcattacgtttaagagtaatattccaagatgaccgtttcaatgaggtttatgataatatcggtaatatcttcacaatcaagtataaaggagaagaagctaacgcaactttttctgtagaacatgatgaagaaactcaaaaagcaagtcgtttagtattaaaagttggagaccaagaagttaagtcatatgatttaactggcggagcttatgactatactaatgctattattacagacattaatcaattacctgatttcgaagctaaattatcacctttcggagataagaacttagaatctagtaaattagataaaattgaaaatgcagatattaaagacaaagctgtatatgttaaagcagttttcggtgacttagaaaaacaaacagcatataacggtattgtatctttcgagcaacttaatgcagaaggagaagtaccaagtaatgtagaggttgaagcaggagaagaatcagctacagtaactgctacttcacctattaaaactattgagccgttcgaattaactaagttaaaaggcggtactaacggtgaaccacctgctacatgggcagataaattagataaatttgcacatgaaggtggatactacattgttccattatcatctaaacaatcagttcatgcagaggtagcttctttcgttaaagaacgttctgacgcaggagaaccaatgagagctattgttggtggaggattcaatgaatctaaagaacaattgttcggtagacaagcatcattatctaatccacgtgtatcattagtagctaactcaggtacttttgttatggatgatggacgtaaaaaccatgtacctgcttatatggtagctgtagctttaggtggtcttgcaagtggtttagaaatcggtgaatcaatcacattcaaaccattacgtgtaagttcattagaccaaatctatgaatcaatagacttagatgaattaaatgaaaatggtattattagtatagagtttgttcgtaaccgtactaatacattcttcagaatcgttgatgacgtaactacattcaatgataaatcagacccagttaaggctgaaatggctgttggggaagctaatgacttcttagtaagtgagcttaaagttcaacttgaagaccaatttattggtactcgtactatcaatacaagtgcttcaattattaaagactttatccaatcttacttgggtcgtaagaaacgtgataatgaaattcaagacttccctgctgaagacgtacaagttattgttgaaggtaacgaagcaagaatttcaatgacagtttacccaatcagaagcttcaagaaaatctctgttagcttggtttacaagcaacagacattacaagcctaatctaggtgatggagtacctggattaggtactcctattaatataatttgaatactttaggagagtgaatacagatggcatcagaagctaaacaaaccgtccatactggtaataccgtcctacttatgattaaaggtaaaccggtaggaagagcacaatcagcatcaggtcaacgtgaatacggtacaactggtgtatacgaaatcggttctatcatgcctcaagaacacgtatacttacgttatgaaggtacaattacagtagaacgtttacgtatgaaaaaagaaaactttgcagatttaggatatgcttcacttggtgaagaaattcttaagaaagatatcattgatattttagttgtagataacttaacgaaacaagttattatctcatatcatggttgctctgcaaataactacaatgaaacttggcagacaaatgaaattgtaacagaagaaatcgagtttagttacttaacagcaagtgacaaagcacgtacttaatatactagaccaactaaaaagttggtctttttttattgacattataaaatttatatgatattataataaacaggttaggagtaatgagtatgaataatagacaagctaaaataaaaggttataatcaatttcattattatgattttccaacaactaaaggtaagtttaaagatataatgaaaagaaagtctagaacagaacttaaaaaagatttacaaaaagagaggaaagattatcttgacaaataagagaaaaacaataggaaaaatgagtaatacgagagcaacatggaatatcaaccctgtaactaaggttaaaaaagataaaacaaaatattctagaaaaaataagcataaaggtcttgacaactataattaactaaggtatattattagtataacaaaaaaggagatgaattacatgagtacattttggtcaaaaagaagaacaacaggtaaggacagacaagtaaagaaacattatactcaaatgtctttacaagaaaagaagaagtgtgttaatctattacaagatacaattaatacgcataagtatttagaactatcaagtcattgtaaaactaaacttaaaaataaaattaattttagtaatctagtaagatttatttttaaggatagaaatgcaccatttaatattattgagtataatattacagattttcatggtaaagaacaaagaagaataatttttaaaagccctactattgtaactatagaaggagtaagtagttatcagtacttagttattaacttagaagatggtacaataataactacatactataatggtattacagatacacataaaacattagatttaaaatattataataaaaacttgacaattaaataaggaggaattataatgggattaatatttgtaaatagttattttataatatcaaacatattaattattgtgtttagtaaattaaatagtaaaaaaaaggtaacagaaaaaactctagctacaagtaatcttttattaacaatatcatatattcaactattagcatttttaatcattaatggtatttactactcattaaaatatatgtaaaaaaagtattgacagaataaaaataataaactataatacagatataacaaattaaaggagaagatataaaatgtcacaagataaattaagagcaatttacacagaaatgaaagtagaattacacaaatttcctaaagaggtagatgtaacaagtaaatcaactgcaattgcaatcaatcagattctagataaatttaaaacattaactgaacaagcagggaaaattacgagaaaatatctagaaggtcaagaaatattaactattgattatgagtattatgattcattacaagaatactatatctacctacttagaaacagtgaaaagattgaacaaagtttacaagaaatcactaagcgtacaggtgaatatgtaaagtaattttgatttaaaaacaaaatatgatatactatgtttaaagtagtaagcctacactagtccgtgttatattaatattgaatcggataagcgtaggctttattaatatttaaaaaaggaaggtatatcatattatggcagaagaaattaaaaaggaacaagatgtacaagaaacaactaaagaagataaaaaagatgttagtaaaatgacaccggaagaaatagataaattaaaatatcaagataagcaagaaaaagaacaagtaattaacaaagttattaaaggtgttaatgatacttgggaaaaagaatataactttgaagaattagatttaagatttaaagttaaaattaaattacctaatgcacgagaacaaggtaacatatttgcgttacgttctgcttacttaggtggtatggatatgtatcaaacagaccaagtaattagagcataccaaatgttagctacattacaagaagtaggtattgaagttcctaaggaattccaagaccctgatgatatctataacttatatcctttaactgttatgtatgaagattggttaggattcttaaactcctttcgttactaatagtatagaaacattagataaagatatagaacgattgggtggtatggaatcaattgttaaacaacctttatctagaaatctatgggctattatgaaagagtttaatgttttgcctactgagcaaagatttaaggatttagacgattatcaaatagagtttattattggtaatatgaatagagatgtttatgaacataataaacaacttaaacaagctcaaaaaggtggaaaattcgatagtcaatttgaagatgacgatagtagttggtggaatgaatctcatgaagactttgacccggtacctgatttcttagatgccgatgacttagcacaacagatggaagctaaattatctgatagagataaggaagaaagagctaagagaaatgatgcagagttaaatgatgaaacagaaggactcactacacaacatcttgctatgatggaatacattagaagaaaacaagaagaattagatgatgaagtaggaaatggtaaaactagtgaagatgatgctactataacacaagagagcgttaataaagcactagaagacctagatgatgactggtatatgtaaagggtggtaggtgatactaccatccttatttttttaaaatggatggtgaataatgatggcaatgaatgacgattatagattggtcttatccggtgatagttcggatttagagaatagtctgaaggcaatagaactttatatggattccctagaatctaaaaatattgatgcccctttagacaatttcttaaagaaattaaaagtaattgctaaagaagttaaaaatgtacagaactcaatggataaacaagaaggtaaatctgtcatatcttctaaagatatggatgaatctattaaatccactcaatctgctacaaagaatataaatgaattaaagaaagccttagatgaccttcaaaaagaaaatatatctaaaggtattgcacctgaccctgaagttgaaaaagcatatgctaagatgggtaaagttgtagatgaaactcaagaaaaacttgagaaaatgtcttcacaaaaaataggctcagacgctagtatacaaaatagaattaaggaaatgaaaaccttaaatcaagtaacagaagaatataataagataagtaaagattctagtgctactaaagactatactaaacgtttaagagctaatcgtaatatgactagagggtatatggagcgttcagaaggaacaggacgtttaacatatgaccaaggggcacgagttagaagtgaactaggtaagataggttcttatgaaagtcaaagaaaacaaaatcaacgcaacttaggtcaagcaagagaacaatatagtaattatagaaaccaacaacaagacctgactaaacgtagagctagtggtcaaataaataaagctcagtatgaacaagaattagcttctattaagcaggaaatgaaagctagagaagaacttatatctaactatgagaaattaggagcagagcttgataaaactgttcaatactataaaggttcagttcagaaagatttccaatctagagatgtagaccaacaacgaggaacatttggtagaatggttcaagagcgtttaccatctatcggttctcatgctatgatgggaactacagctatggctacgggtttatacatgaaaggtgcttcattaagtgaaactaatagaccaatggttacatcactaggtcaaaactcagataatatggatatagattctgtaagaaatgcatatggagacttgtcaattgataacaaattaggttataatagtactgacatgttgaaaatggctacttcatatgaagcatctgtaggacataaaagtgatgaggatacaatggcaggaactaaacaacttgctattggaggacgttctttaggtattaaagaccaagaagcttatcaagagtctatgggtcagataatgcatactggtggagtaaattccgataacatgaaggagatgcaagatgcattcctaggcgggattaaacaatcaggtatggttggtcgtcaagatgaacaacttaaagcattaggttctatagctgaacaatcaggagaaggaagaactttaactaaagaccaaatgagtaatcttactgctatgcaatctacttttgcagagtcaggaagtaaaggattacaaggtgaacaaggtgccaatgctattaatagtatagaccaaggacttaaaaatggtatgaatagttcttatgctcgtatagcaatgggatggggaacacagtaccaaggtcttgaaggtggatatgatttacaaaaacgtatggatgaaggtatatctaaccctgaaaacttgacagacatggctgatatggctactcaaatgggtggtagtgaaaaagaacaaaaatacctattcaatagaagtatgaaagaaataggtgctaacttaactatggagcaatctgatgagatatttaaagatgctcaatccggaaaattatctaaagaagagttagctaaaaaagctaagaaaatggaaaaagaaggtaaaaaagaaggagaagataacgccactgattataaagaatctaaatcaggaaaaaatgaccaaaataaatctaagactgatgataaggcagaagatacttatgatatggctcaaccattaagagatgctcatagtgctttagcagggctacctgctcctatatatttagcaataggagctattggagcatttacagcatcactaattgcatctgcaagtcaatttggggcaggtcatttaataggtaaaggagctaaaggacttagaaataaatttggcagaaataagggtggtagctccggaggtaaccctatggcaggaggaatgcctactggaggaggttcacctaaaggcggaggctctcctaaaggtggcggtactcgttctactggaggtaaaatacttgatagtgctaaaggattaggaggattcctagtcggtggagcaggatggaaaggtatgtttggtggagaatctaaaggtaaaggatttaaacaaacatctaaagaagcctggtcaggtactagaaaagtatttaacagagacaatggtagaaaagccatggataaatctaaagatatagctaaaggtactggtagcggtcttaaagatatttataatgatagtatatttggaaaagaaagaagacaaaatctaggagataaagctaaaggttttggtggaaaagctaaaggtctctatggtaaatttgctgataagtttggtgatggtggtaaaaatggtatcctttcacaatcaccaaaagcaggcggtagcgggctaggaaaactaggaaaacttgcaggtggacttggaaaaggagccggagttttaggtgttgctacatctgccttatcattgatacctgctttagcttccggtgatagtaaagctatcggcggaggtataggctctatgggcggaggaatggcaggtgcatcagcaggagcttctattggagctttatttggtggtgtaggtgcaatacctggagctttaataggcggagctataggttcttttggtggaggagctgttggtgaaaaagtcggagacatggctaagaaggctaacactaaagaaggatggaacctaggatggactaatggagataaagacggtaagaataaattccaagattctttattaggaaaacctatatctaaagcatggagcggtataacaggtctctttgataatgacgctgaagcatctgaagaagatagcaaagataagaaaaaaggcgttaaaggtgttaaaggagatactaagaagaaagaaaaaatgacagcagaacaacttagagaaaagaataaccaatctgaaactaagaatcttaaaatctatagtgatttacttgatagggctcagaaaattattgagagtgctaaaggtattaatatagatggaggaacttctgatagtggttctgatagcggaggctctgcatctgatgtaggtggagaaggcgcagagaaaatgtataagttccttaaaggaaaaggactatctgacaatcaggtaggagctgttatggggaacttacaacaagaatctaatcttgaccctaatgctaagaatgcctctagtggtgcatttggtattgctcagtggttaggggctagaaaaacaggattagaaaactttgctaaatctaaaggtaaaaaatctagtgacatggatgttcaattagattacctatggaaagagatgcagtctgattatgaaagtaataatcttaaaaatgccggatggagtaaaggcggaagcttagaacagaatacaaaagcatttgctaccggatttgaacgtatgggagcaaacgaggctatgatgggtactcgtgttaacaatgctaaagaattcaagaagaaatacggaggctccggtggcggaggaggtggaggagctctatcctctacttaccaagaagctatgagtaaccctgtattaactactggttctaactataaaggctctaatgatgcttctaatgcttctacaactaatagaataacagttaatgttaacgttcaaggtggaaataatcctgaagaaactggagacattatcggaggaagaattagagaagttctagacagcaacatggatatttttgcaaatgaacataagagaagttactagtaattttgtattgacacaagagtagtatcatagtatactactcttatacatataaaaataaaaggaagtatgtgtatatgaaaagattaagaagacctaaggtaagaatagagatagttacagatgataatacatttacattaagatttgaagatacacgtgactacaatggtgatgagttcggagctaaacttttaggtttccaaactaaaaactctatggaagatgatagttctgtattccaaatcaatatggcaggagatacttactgggataagttagttatggctaacgatataatcagaatatttattacacctaatgatgaccctaatgataaagaaggtcgtcaagaacgtttaatccaagtaggtatggtatcacaagtatcaaaagtaggtagttatggtaatgaccaaactcaatttaggataacaggtcaatcttttgtaaaaccttttatgaaatttggattaggagttattcaggaagttcaagctgtattacctgaagtaggttggcttattgatggtgacggagataatgaagtaaaatttactggtagctcagcccatgaagtaatgacaggaattatacgtagattcataccatatatgaagtataactatactgaaaaaacatataatacaattgataactatcttgattatgatgatttaagtagttgggatgaatttgaaaatttgacagaagtatctgcttttactaattttgatggctcattaaaacagttgatggatatggtaacagctagaccctttaatgaattgttcttcaaaaactctgaaaaaacacctggtaaagcacaacttgtattaagaaaaaccccttttaatcctactgagtggagagctttagatatgattaaagtacctactgaagactttattgaagaagatgtaggtaaaagtgacgtagagacatattctatatttacagctacacctgcaggtatgttaaaagaacttaatggtgatgtattttctaaaccacaatttcaccctgaattgactgatagatatgggtatactaaatttgaagtagaaaatatctatcttagtactaaatcaggttcagctactgaagactcagattcttcgggtgatgacaatggtactgaaagaggaacttactctaaaattatgaaagatttaagtaactatggaagagataatatatctaaaggtatagataagtatacaagtaaattatcttcaaaatataaaaacttaaaaaaagctcaagctaaaaaaattatagagaaatttgtcaaagaaggaaaagtaacagaaaaagaatacgagaagataacaggtaataaagtagatgatgaattaacatcagataacagaccgaagttgacaaaggataaattaaagagtatactaaaagagaagtttaaaacacaagatgattttaataactctaagaaaaagaaaaaagctaagacagatgcacttaaagaattgacaactaaatatcgtttcggtaacaaaacacacgctacaactttgttagatgaatatattaaatacaaaggagaaccaccgaatgatgaagcttttgataaatatcttaaagctattgaaggtgttagtaacgtagctacagacacaggttcagatgcaagtgatagtcctttagttatgttctctagaatgctatttaattggtatcatgggaaccctaacttctacgcaggagatattattgttttaggagaccctaagtatgacctaggtaaaaggttatttattgaggataaacaacggggagatacttgggaattctatattgagtctgtagaacataaatttgattataaacaaggttattatacaactgtaggagtaactagaggtttaaaagatgctatcctagaagatggtaagggtagtcctcataggtttgcaggtttatggaatcaatcatcagacttcatgggaggtcttatgggtgaagatacttctaaagaactcaaagaaaaaggtgtagcagagaagaaaagtagtggagataaagacggtggctctgatagtggtggagcacaagatggaggttctctagattcacttaaaaaatataatggtaaacttcctaaacatgacccaagtttcgttcagcccggtaaccgacattacaagtatcaatgtacttggtatgcttataatagaagaggtcaattaggcattcctgtgcctttatggggggatgccgccgactggattggtggtgctaaaagtgcaggatatggagtaggtaagacaccaaaacaaggtgcctgtgttatttggcaacgaggagacaaaggtggttctaatgcttatggtcacgttgcctttgtggaaaaagttttagatggtgggaagaaaatattcatttctgaacataactgggctacgcctaatggttatggtactagaacaatagacatgagctctcaaataggtaaagaagctcaattcatttacgataagaaataaaggaggatagtctatggcaacagataaagaagctaaagatgttattgataaatttatagataatgtgtttaattttgatgtattaactacggaaagagttaaagaaaaagatgaagaaatcaaaaaaataactacagatgatatgtatgaaaaagttgtgtatatacgaccttatgttggagtaatacaaagtcttaaccctcaacatgtacaatatgaatcattttctaataatggttacgatatagaagcagaattaagttttaggaaagtaagttatttagttgataaagggtctatacctacagattctttatccactttaacagttcacttagtagaaagaaatcaggagttattaatagattactttgatgagatacaagatgtgttgtatggggagtatatggaagaagaatatgtatttgatgaagatgtaccattaagtactatactagcattagacttaaatgataatcttaaatccttatcaaatataaagtatatgttcaaaggagcccctaaagataatccttttggaacagataaagatgtttatatagatacttataatttattatattggttgtatttaggtgaagatgaagaattagcatatcctatgaatattaactatttctttacagaaggtagattttttactgtttttggtaaaggtcataagtataaagtagatgttagtaaatttatagttggagatatattattctttggtagaagtgatactaatataggtatttatgtaggtgatggagagtttatatctatgataggtaaatttccaaaagatgagacacctataggaaaatacaaacttgatgattactggaatgaatttaacggaagagttatgagattcgatgaagaggtgtatatttaatggtagtaagattccaatcttccatggggagaagtctaaaaagagtagattcagatgacttaaatgtaaaagggttagttttagctacagttagtaaaattaattataagtatcaatcagtagaagttaaagttaataacttgactttaggaagccgtataggtgatgatggtagcttagctgtaccttatcctaaatctttcataggtagaacacctgagggaagcgtattcggtacaaaaccacttattactgaaggttctgtagtattaatagggttcctaaatgatgatataaatagtcctataatcttgagtgtttatggtgataatgaacaaaataaaatgattaatactaaccccttagatggaggtaagtttgatacagaaagtgtttacaaatacagtagttcactatatgaaattttaccatctttaaattataaatatgatgatggagaagggacaagtattagaacttataatggtaaatcattcttctctatgacatcaggtgaagaagagaaaccgcaggcaacagatttttatactggaactgagtatcaagatttatttacttcctattatggtaataaaacattgattgaacctagaatacaaaaggctcctaatatgttattcaaacatcaaggagttttttatgatgatggcacgccggataatcacataactactttatttatatctgaaagaggagatatcagagcttcagttttaaatacagaaacacagaaaagaactacacaagaaatgtcaagtgatggttcttatagggttattaaacaggatgacgatttaatgttggatgaggctcaagtttggattgaatatggtattagtgaagataataaattttatattaaaaatgataagcataaatttgaatttactgatgaaggaatttatatagacgataagcctatgctagaaaatttagatgagagtatagcagaggctatgaagaatttaaatgaaatacaaaaagaactagatgatataaactatcttcttgagggtgtaggtaaggataatttagaagaactaatagaatccacaaaagagtctatagaagcgtctaaaaaagcaacttcagatgttaatagacttacaactcagatatcagaggttagtggtagaactgagggtattataactcaattccaaaaatttagagatgagacttttaaagacttttatgaagatgcttctactgttattaatgaagtaaatcagaatttccctagtatgaaaacagatgttaataatctaaaaactaaagttaataacttagagaaaactgagataccaaatatcaaaaataggttaacagaactagagaataataataacaatgcagataaaataatttcagatagaggagagcatataggtgctatgatacagttagaggaaaatgttactgtaccaatgagaaaatatatgccaataccttggagtaaagttacttataataatgctgagttttgggattctaataaccctactagattagtagtacctaagggaataacaaaagtaagagttgcaggtaatgtcctatgggattctaacgctacaggacaacgtatgttgagaatattgaaaaatggtacttatagtttagggttaccttatacaagggatgtagctatatctaccgcacctcagaatggtactagtggagttattcctgttaaagaaggagactactttgagtttgaggcttttcaagattcagaaggtgacagacaattcagagcagacccttatacatggtttagtattgaagctatagaattagaaactgaaaccatggagaaagactttatgcttataggtcataggggagcaactggatatacagatgaacatacaataaaagggtatcaaatggctttagataaaggtgcagattatatagaattggatttacaattaacaaaagataataagttattgtgtatgcatgattctactatagatagaacaacaacaggaacaggtaaggtaggggatatgactttatcttatatacaaactaactttacatctcttaatggtgagcctataccatctcttgatgatgtattaaatcattttggaacaaaagttaaatactatatagaaactaaacgtccttttgatgctaatatggataaagaattattaactcagttaaaatcaaaaggattaataggaattggttcagaaagattccaagtaattattcaatcatttgctagggaatcattaattaatattcataatcaattctctaatatacctttagcttacttaacaagtacattctctgaaagtgaaatggatgattgtttaagttatggttcttatgctattgctcctaagtatacaactataactaaagaattagtagatttagcccatagtaaaggacttaaagtacacgcatggacggtaaatacaaaagaagaaatgcaaagcttgatacaaatgggtgtagatggattctttacaaactacttagatgaatataaaaagatttaatattaaagacctattaatttaggtctttttttagttgtaatttaaactagttcgtgatatattagtagtatgagatttatatacatactgaaaaggagaggataaaatgccacaatcagatggaataagtaatcttcatagaatagctttacgcttccctaaagaaggcggtggttatgatatgtatagatttaaagttaaccctgagaactacacaatagattcaccacaacgtacgacagcaattaaaacaaaatcagatattgtaatagaagattatggtaaagacatagaagttattaacttcacaggtacaactggttttagacctgttagagaagcagacggattaaaaacaggtaagcagaaaatggaagagttacaaagtagagttagtgaatatgctatgcaaggtggtagtggtaatgtaagtggttcttacttacaattttttaactttacagatgatagctactataaagttcatttagctcctcaagggttaaagataactaggtctaaagatgaaccattactttttagatatgaaataacattagtagttattggttcgttaacagaagcagatagaagtgctgtaacaacagaagagtttggtaatgttaaacctaatgcttctcaaagagtagatgagggtataaaagaattagataaaaatgctcgtaaaacgagagatagaaataatcaagaaatatctaaaagagaaaatacaatacctaaatctacaggagataatacgaatgagggtaatagacttaagcaaagcttccctagtagttctatatataatcctagacaatctactaacggattaaaagggaatattgacaatatggctctgataataggttacggtgatggaggtgtatctagctaatgaataattttataccacaacctcaaggtctactcagatttttaaatgccctagatacagatttaacttcttctcacatgaatttactggatgaagaggtatcatttgtatctaaattttacacaccacagctacaattaagtgaattagcaaaaaaagtattgacaaatataaagacagatgatatacctgtattagaaagagaatttaatgataatacaattatccataaagctaatgatacattactaaaagtacaggctccaagaatgtatatgattctacagtctattgtgcttgaagcatatgctattgttaattgctttgtagaaaatccaagctctttaaaatacttaactgaagaagatgttagtataacacgagaaaacttaaattatgtagctgactacttaggtaactatgatgactacaatagtgttgtcttagacttaagagatttagacttatgttttagtgcgatagaattacaattacctctaattaaaaaggaggctaatgtataatgagatttaagaaacacgtagttcaacatgaagaaacgatgcaagcaatagcacagagatactatggtgatgttagttattggatagacctagtagagcataataatttaaagtatccctatttagtagaaactaatgaagaaaaaatgaaagaccctgaacgattagcttctacaggtgatacactgattatacctatagaatctgatttaacagatgtatcagcaaaagaaattaattctagggataaagatgtactagttgaattagctttaggaagagatttaaatattactgcagatgaaaagtattttaatgaacatggtactagtgataatatactagcattcagcacaaatggtaacggagatttagatactgtaaaaggcatagataatatgaaacagcaattacaggcacgtttattaactcctaggggttccttaatgctacatcctaattatggttcagatttgcataatttatttggtcttaatatacctgaacaagctacattaatagaaatggaagtattgagaacattaacatcagataatagagtaaaatctgctaatctaattgattggaaaatacaaggtaatgtttattcaggtcaattttcagtggaaataaaatctgttgaagaatcaataaattttgtcttagggcaagatgaggaaggaatttttgctttatttgaataggaaaggattaaattatgaaaactagaaaattaactaacatactatcaaaattaatagataagacaatggcaggtacaagcaagataacagactttactcctggttcagcttctcgttcattattagaagctgtatcattagaaatagagcaattctatattctaacaaaagaaaatattgattggggtatacaagagggtatcattgaagcctttgattttcaaaaaagacaatctaaaagagcttatggtgatgttactattcaattctaccaacccttagatatgagaatgtatatacccgcaggaacaacttttacttcaacacgacaagaatatcctcagcaatttgaaacattagttgattattatgcagagcctgattctactgagattgttgttgaagtttattgtaaagaaacaggggttgcaggtaatgttcctgaaggaacaattaatactatagcatcaggttctagtttgattagaagtgttaataacgagtattcttttaatacaggaactaaagaagagagccaagaagactttaagcgcagattccactcttttgtagaatctagaggtagagcaactaataaatcagtaagatatggtgcattgcagatacctgatgtagaaggtgtttatgtttatgaagaaacaggacatattacagtatttgctcatgatagaaatggtaatttatcagataccttaaaagaagatataattgatgctttacaagactatagaccaagtggtataatgttagatgttacaggtgtagaaaaagaagaagttaatgtttctgctacagtaactatatctaataaatctagaattggtgatacattacaaaaacatatcgaaggtgttattagaagctatttaaataatctaaaaacttctgatgacttaataattacagaccttattcaagctataatgaatattgatgatgtactaatatatgatgtgtcatttgataacctagatgagaacattatagtaccaccacaaggaattattagagcaggagaaataaaagtagaactaaagtaaagagaggtgaaacttaagtcgtggctaattttttaaagaatcttcatccattattaagaagagatagaaataaaaaagataatcaagaccctaactttgctctgatagatgcactcaatgaagagatgaatcaagtagagaaagatgctatagaaagtaagttacaatcttctctaaagacatctacaagtgaatatttagataagtttggggattggtttggagtttatcgtaagacagatgagaatgatgatgtttatagagcaagaattataaaatatttactcttgaaaagaggaactaataatgctataatagatgctataaaagattatttaggtagggatgatattgatgtaagtgtatatgaaccttttacaaatattttctatactaacaaatcacatttaaatggtgaagaccacttaatgggatactattatagatttgctgttattaatgtctctataggtgattatttccctgtagagattatagatgtaattaatgaattcaaacctgcaggtgtaactctgtatgtcacttatgatggagcttctactattagaggtggagcaattattaagtggttagatgggttacctaaaatagaaacatatcaagagtttgataggtttacaggatacgatgatacattctatggtcatattaacatgaatcaaagtaaagatactgataatagtacatcagatatttttaaaacaaaccatagcttaattaatagtttagatgttttaacaggttcctctagcgtaggtagacagtatgttaactatggatatataacgtcatatgtttataatccaggtatgacatcttctgtaaatcaaataagcgctagtacagaaggtagagggcaagaagtacctactgactattatatgtatactagtactaagaataacaatacagtagaacttagtatgcaaactacttccggtgtgtcttatttatataataactttaattttagggattatatgagtaaatatagacctcaagtaaatttacaatctgatgaggatagaagaattgtatctgattatataaaagaattaagtattgattattatctcagtgctgtaatacctcctgatgaaagtatagaaattaaattacaagtttatgatttttctattaatagatggcttacagtatcaattaataatttatctttctatgaaaaaaatatcggtagcaatataggatatataaaagattatttaaacagtgaattaaatatgtttactagattagagataaacgcaggtaaaagagattcagtagatattaaagttaattacttagatttaatgttttattattatgaacgaggtatttatacaataaaaccttataaagccttagtagaaaattatttagatatatctagagagacttacgtagaggcatttaaaatagcatcactatctaatggagatattataactaaaacaggctatttacctataggttatctaagagtatcaggaggtattgataacctaagtaaccatatagaaattattactacagataataacactaatagtattccaagtactattttagaagataactctaatagtttgatattatcgtatggtaacgtcaaaaccaatatacacagttttgaattaaatagtgatgtttcaatttcaaatattaaatttgaatactcttattatggtgatgcttgggaagaactgacagcattaactgaaatatctgagggtgaaactatagtacctaatatattaatagatttatatggattacagacagtagattattctaatataaatccaatgtcaaaagtgtcattacgttctatttggaatgttaagctaggtgaattgaacaatcaagaaggttccttatctaatatgcctaatgattactttaatgctgtatggcaggatatagataaattatcagatattgagttaggttctatgagaatgattaaagacactgagggtggagtatttgatggagctacaggtgaaattattaaggctactctatttaatgtcggtgcttatactgatttagacatgttagcttatactttgactaactatactgaaccattaacgttaggctctagtcgattaataagtgagctaaaagaagaactattaacatcagaatcatttaatgtcgataatagaattaaagtaattgactcaatatctgagcagttacctaataacaatatattaagtaactcttaccaaacacaaactattacacagaatggatttgctaagtataatttgaaagaacctatagagcagagaaaacaatacaatctaagaatacatggagattttaaagaagggttagaaagattagctataggcaattctaatggttcatttaatgaagtatttgtttaccctgaaaatattaaagatggtatagtagatattacttacacttctagagatgataattacgcagaaggaaaacaaaggcttaataatgattatagagtttacgctcaaccatacgatagtaaagtagtaacaatttacagtttagagttaataaaagtttaataaataagttgacagaaagttaataatatggtatacttataaagtaatatttagtgggtataccatgttatattaataaagaaaacaacagatgaaaggaattaaaaaatatggcaattgcaacgtataattctcatgttgagttagcaaaatatctagttagtaaagctgattcagtttacttaacaattggaaagagcacaccgtggtctaatgaaacaaacccaccgcaacctgatgaaaatgcaacagtattacaggaggttataggatacaaaaaagctactaaagtaactttagttagaccttctaaatcacctgaagatgataataagaatttaatttcttatggtaataaatcatgggtagaagtaacacctgaaaatgctaaagatgaaggagctaaatgggtttacttagaaagcagtattgttggtgacgaactacctcttggaacatatagacaagtaggatttgttatggacttagtagcaaaaagtggtattagtaaatttaacttagtacctagtgaagtagaatcaattggaacattgttattctttgataataaacaattccaaaatagaagtgagcaaacaactgctaaagaaagatttattgtagaagtttaaagaaagggagataattctaaatggcaattaattttaaaggttcaccttatttagatagatttgacccgtctaaagatagaacaaaagtattatttaatcctgatagacctctacaacaggcagaattaaatgaaatgcagtctatagaccaatattatttaaaaaatctaggagacgctatttttaaagacggagataaacaatcaggtcttggattcacattatctgaagataatgtattgacagtaaatcctggttatgtatatatcaacggtaaaataagatattacgataatgacgattcagttaaaataactggcgtaggtaaagaaactatcggtattaagttaacagaacgtattgttacacctgatgaagatgctagcctactagaccaaactagcggagtaccaagttacttctctaaaggtgcagatagattagaagagaagatgtcattaactgttaatgaccctacatcagcaactatttatactttcatggatggagatttatatatccaatcaactaatgctgagatggataaaatcaataaagtattagctgaacgtacttatgatgaatcaggttcatataaagtaaatggtttcgagctattctcagaaggtaatgctgaagatgatgaccacgtttctgtagttgtagatgcaggtaaagcttacgtaaaaggttttaaagtagataaaccagtatctacaagaattagtgtacctaaatcgtatgacttaggaacagcagaaaatgaaagtactatctttaataagtctaataattctattagtttagctaatagccctgtaaaagaaattagacgtgttacaggtcaagtacttattgaaaaagaacgagttactagaggagcagaaggtgatggacaagacttcttatctaataatacagcgtttgagattgtaaaagtttggactgaaacaagccctggagttactacaaaagaatataaacaaggagaagacttcagattaacagatggtcaaacgattgactggtcacctcaaggtcaagaaccttcaggaggtacttcatactatgtttcttataaatataataaacgtatggaagttggtaaagattatgaagtaacaactcaaggtgaagggctaagtaagaaatggtatattaattttacacctgaaaatggggctaaacctattgaccaaacagtagtattagtagattatacttactacttggctcgtaaagattcagtgtttattaataagtatggtgacattgcaatattacctggtgaacctaatattatgagattagttacaccaccattaaacacagaccctgagaatttacaattaggtacagttacagtattacctgattcagatgaagcagtatgtatttcatttgcaatcactagattgtctatggaagacttacagaaagttaaaacaagagtagataacttagagtataaccaagcagtaaatgctctagatgatggtgctatggaaggacagaaccctctaacattacgttcagtatttagtgaaggtttcattagtcttgacaaagcagatattacacatcctgacttcggaattgtatttagttttgaagatgcagaagctactctagcttatacagaagcagttaaccaacctaagattattccaggagataccacagctcatatttggggtagattaatttcagcaccatttactgaggaacgtacaatctatcaaggtcaagcatcagaaacattaaatgttaacccttataatattcctaacaaacaaggtgtgttaaaattaacacctagtgaggataactggattgatactgaaaatgttacaatcactgaacaaaaaactaaaaaagtaactatgaaacgattttggagacataatgaaagttactatggtgagactgagcattacttgtattctaacttacagttagatgcaggacaaaagtggaaaggtgaaacttacgcttatgatagagagcatggtcgtactggtactttattagaatcaggaggacaacgtactctagaagaaatgattgaattcattagaatcagagatgtatccttcgaagttaaaggactaaaccctaatgataataacttatatttattatttgatggagtaagatgtgctataacacctgcaactggttatagaaaaggctctgaagatggtacaataatgacagatgctaaaggaacagctaaaggtaaatttactattcctgcaggtattcgttgtggtaaccgagaagttacccttaagaatgctaactctacaagtgctacaacttacacagctcaaggacgtaaaaaaatcgttcaagatattattatcagaactcgtgtaacagtaaacttagtagacccgttagcacaatcattccaatatgatgagaacagaactatatcatcattaggattatactttgcttctaaaggtgataaacaatctaatgttgttatccaaattagaggtatgggtgaccaaggttatcctaataaaacaatctatgcagaaacagttatgaacgctgatgatattaaagtatctaataatgctagtgctgaaactagagtatactttgatgaccctatgatggctgaaggcggtaaggaatacgctattgttattattactgagaacagtgattatacaatgtgggtaggtactagaactaagcctaaaattgataaacctaatgaggttatctcaggtaacccataccttcaaggtgtattattcagttcatcaaacgcatcaacatggactcctcatcaaaactctgaccttaaatttggtatttacacttctaaatttaatgagacagcaacaattgaattcgaaccaattaaagatgtatctgcagatagaatagttcttatgtctacgtacttaactcctgagagaacaggatgtacatgggaaatgaaaataattctagatgatatggcatcttctacaacattcgaccaattgaaatgggagcctattggtaactatcaagatttagatgttttagggctagcaagacaagttaagttaagagcaactttcgaatctaatagatatatctcaccattaatgagctccagtgatttaacattcactacattcttaacagaattaacaggttcatatgttggtagagctattgatatgacagaggctccttacaatacagtaagatttagttatgaagctttcttacctaaaggtactaaagtagttcctaagtactctgatgatgatggaaaaacttggaaaacatttactaaatcccctacaactactagagccaataatgagtttacacgctatgtcattgacgagaaagtaaaatcatcaggaacaaatactaaactacaagttagattagatttatcaactgaaaatagctttttacgtcctcgtgttcgtagacttatggttactactagggatgaataaactagaggggttgattgacccctctttatttaataaggagagatttatatgcctagagaagttagagacccttattctcaagctaaattatttatacctacagttgaggaaaaatcaattaaggaattagaaaaaacatacaaagaaaaaattgatgaagctactaaattaatcaatgaattaaagaaagagagaggagaaaaatagatggcatttaactacacgcctcttactgaaacacagaagctaaaagacatgtatcctaaagttaatgatataggtaattttttaaaaacagaagttaaccttagtgatgtaaaacaaatatcacagcccgactttaataatattttagcatctatacctgatagtggtaactattatgtaactaattcaaaaaatgcacccagtggagaatctacggcaggatttgtaatactggataaaagaaatgtaaattattataaaatttactattcaccatatagcagtaataaaatgtatatcaagacttatgctaatggtactgtatatgattggattagttttaaattagatgaaggtaacctctacgatgaaggtaatactttaaatgtaaaggaacttactgaatctacaacccaatatgcaactcttattaaccctccaaaagagagcttaaatacgggatgggttaattacaaagaaagtaaaaacggtgtttcttctttagtagaatttaacccggttaactctacctcaactttcaagatgataagaaagttaccagtacaagaacaaaaacctaacttattgagagatagtttatttgtttatcctgaaactagttcttcaaatatcaaaacagataattggagtacgcctcctttttggggatacacagctaatagcggtcgttcaggggttagatttagaggggaaaatactatacagcttgatgatggcagtagtacctaccctacagcaatgactaatagatttaaaatgggtaacgagctatctgtaggagatacaataactgtatctgtatatgctaaaattaatgacccaacgttacttaaagataataaagcatactttgagatagcggggtatgatacggtagatagagctgataatccttatacaggaggacgtagagaaataacagcaagtgagataacaactgagtggaaaaaatactctttcacattcacgatacctgaaaatacaataggtgaatcaggtgcaaaagttaattatatttcattactcttaagaatgaattgttcatctagtaaaggtaatggtgctgtagtatactatgctctacctaaattagaaaaatcatctaaagttacaccgtttatcacacatgcaactgatgttcgtaaatatgatgagatttggtctaactggcaagaagttattagtaaagatgaattaaaaggtcactctcctgtagatatagaatataatgattactttaagtaccagtggtggaaagctgaagttaatgaaaagaacttaaaagatttagctatgacagtacctcaaggttatcatacattttattgtcaaggttctattgaaggaacacctagaggacgttctattagaggaactattcaagtagactatgacaaaggtgacccatatagagctaataagtttgttaaactattatttactgacacagatggtataccttatacattatactatggagggtacaaccaaggttggaaacctttaaaacaacaaagaacatctacaatactttgggaaggtactttagactatggttcaaaagaaaatataactttaaatgacccttggactaactatgatatgttagaagttgtgtatctaacacaatcagcaggtcactataaaacaatgtttttagacttaagaacgcctacaacgccatatttatatataagagacttcaatctagctaatagttctacaggctcaggggtagacttttttgaagggtatctatctttcccaacaacaacttcagctacacctactatggttaaaaaagttactttaaatgggtctacaaatacgacatctgtatctgatttcaatgctcaaggaataataactatatatagaatagcaggaattaatacactttaggaggataaataggatgactagacaatttgatattggcagagatgagatagtattacatctaagagaaggtaaatatattacagggttcacaacagcaggggcttatgatacagattttgggcagataaaagttaatagagaaattttacctgcttacttctttgataattttgcctatgaaagatacttgtattatagtaaacctgaagaggttatagaaaataaagactatgtaccacctcaaatcaataatggtgatgaggaatctcaacaaaatactgtacctaaagaacaatatgatagtttaaaagaagaactagaacttatgagaaaacaacaagaagctatgatggaaatgcttcaaaaactcttaggtcaaaaggggtaataataaatggcattaaattttactacaataacggaaaacaatgttattaaagacctgactactcaggtcaataacattggagaagaattaacaaaagaaagaaatatatttgacattacagatgatttagtttataattttaataaatcacagaagattaaactaactgatgataaaggattaactaaatcgtatggaaacataacagctcttagagatataaaagaaccaggttactactatataggcgctagaacattagcaacattattagatagacctgatatggagtctcttgatgttgttttacatgtagtacctcttgatacttctagtaaggtagttcaacatttatatacgctatctactaacaataaccaaattaaaatgttatataggtttgtctcaggaaactctagttcagaatggcaatttattcaaggattaccgagtaataaaaatgctgttatatcaggaactaatattctagatatagcttcaccaggtgtttattttgttatgggaatgacaggagggatgcctagtggtgtagattcaggttttttagatttaagtgtagatgctaatgacaatagattagctagactaactgatgctgaaactggtaaagaatatactagtattaagaagcctacaggagtatacacagcttggaaaaaagaatttgagccaaaagatatggagaaatatttactaagtagtatcagagacgatggtagtgcatcattcccactcctagtttatactagtgataataaaacgtttcaacaagctattatagaccatatagatagaacaggtcaaacaacctttactttctacgttcaaggtggtgtatcaggttcccctatgtctaatagttgtcgagggttattcatgtcagatacacctaacacttctagtttacatggtgtctataatgctataggtacagatggtagaaatgtaacaggttcagtggtaggaggtaattggacttcaccaaagacatcaccttcccataaagaattatggacgggagcacaatcattcctatctgtaggtactactaagaatctagcagatgatattagtaattactcttatgtagaggtttatactaaacataagacagtagagaagactaaaggtaatgatgactcaggtacaatttgccacaagttctacttagatggtagcggtacttacgtttgctcaggaacttttgtttcaggagatagaacagatacaaaaccacctgttacagagttctatagagtaggtgtatctttcaaaggttcaacatggacgcttgtagatagtgcagtacaaaatagtaaaactcaatacgttacaagaattataggtattaatatgccatagactaggataagtttcctagtctttttttcttgacttgaaaaggattctatggtatactataactcgtgtaaggatataaggagattaaaatgagattaagaattaaaaacttatatacctatgtagaatttgaggaggatgataaatacttaaaagatatatttttaaagagagtccatacaactataggagcaaggcaagaaggttttcagtatagccctgcttacaaaagaggcagttgggacgggtatgtagacttttatgtttatgaggaagataaattccctactggacttttatttaaaattgagttattattaggtgagctacaatcaaggtataacttccagtttgaaacaattgatgagcgtgatgaaagtttcttatctgaagaagatattgatgatgagataacattgcttgataataatgtaggtcaaattaccttacgagattatcaatatgaggcagtgtacaacagcttaacattttacaatggtattgctcatttagctactaatggaggtaaaactgaggttgctagtggtattatagaccaactattacctcaattagaaaaaggtgaaagagtagcattcttcacaggctctacagagatattccatcagtctgcagatagactacaagaacgtttaaatatccctatcggtaaagtgggtgcaggtaagtttgatgttaagcaggttacagttgtaatgatacctactttaaatgcaaaccttaaagacccaacacaaggggtaaaggttacacctaaacaaaatattagtaaaaagattgctcaagagatattacctaaatttgaaggtggaacaaatcaaaagaaattactaaaagtattacttgataacacaacacctaaaacaaaagtagaacaaaacgtattaagtgccttagagataatttaccaaaacagcaagacggatgcagaagttttattaaacttaagaaatcataatgcacattttcaaaaaattgttagagaaaagaacgaaaagaaatatgataaatatcaagatatgagagattttttagactcagttacagttatgatagttgatgaggcacaccattctaaatctgattcctggtacaataatctaatgacatgtgaaaaagctttatatagaattgcattaacagggtctatagataaaaaagatgaattactttggatgagattgcaggcgctattcggtaatgttattgcaagaactactaataagtttttaattgacgaaggtcattctgctagaccaacaataaatattatacctgtagctaatcctaatgacatagatagaattgatgattatagggaagcttacgataaaggtataacaaataatgattttagaaataaacttattgcaaaactaacagaaaagtggtataatcaagataaaggtacattgattattgtaaacttcatcgaacatggagatacaatatcagaaatgttaaatgatttagatgtagagcactacttcttacatggagaaatagactctgaaactcgtagagaaaaattaaatgatatgagaagtggtaagcttaaagtaatgatagctacatcacttattgatgagggtgtagatatatccggtattaatgcgctaatattaggtgcaggaggtaaatcgttaagacaaacattacaacgtattggtcgtgcgttacgtaagaagaaagacgataatacaacacaaatatttgattttaatgatatgacaaatagatttttatatactcatgctaatgagcgtaggaaaatttatgaagaggaagattttgaaataaaagacttaggaaaataggagggtaagagatggcaacaagaacacaacgaaagttataccaatatctagaagaaaatgctacagaaaataaatttcatatttctactaagaaagagttagcagattctctaggcgtttccatctctgctttatccaataaccttaaaaagttagaagaagaaaataaagttgttactgtttctaaaagaggaaaaaatggtggagtaataataactttagttagggaatatgacacagaagaattgaaagaatttaataattctacagataatatcattacttctgatttacagtatgctaaagcattaagagaaaagcactttccttcttatagatacgagaggaaagaacagcgtagacgtactaagttagaaatggcacagtataatgctattaaagatgaaaagagaagaattatagcagatatgaactttcattcagaaggtcttccttatccttctaaagatatatttaatatgtcttatgacccggaaggattttataaagcatacattctatgtaagttatatgaccaatatgctatttctcatatggatgctaaacatacaagccatcttaaagcaatgagtaaggcaacaactaaagatgaatatgattaccatcaacatatgtctgaatactatagaaataaaatgattcaaaatttacctagaaatagtgttagtgataatttcttcggtagtaaaatgtttaatactttctataatttttatttaaaaataaaagataaaaatattaatgtatttaaatatatgcaaaacgtatttaagaatgtaacattttattatgagaatggtatgcaacctaatccaataccttctcctaacttctttagttcagataagtattttaaaaactataataattatattaaaggaataaaaaaaggtgttaatagtacaaatagacacttaggggatacagatagtattattaattcatcagattatgttaagaaccctgctgtattacatttacatcaattatatactacaggattaaattctactttacatgatattgatactatgtttgagcaagccttagaccttgagaatgcttcttatggactatttggagatatgaaacatattattttactacaatataattctatgattgaagaagaaattaagaacttacctacagaagagaaaagtattattaataaatatgtaaaacaatgcataattaatgattactccccaacaagcatatcaccatctgcaagattatcaatgtttactatgcagaaagagcatatagtttataataaacagttaaacagaggaatcaagagagaagatttattaccattaagtctaggaggtatagtgaataaagattcactaagtggtatggatatacaaaacttagagcagaatggtaatgagtacctatatatgagacaacatacttcaacttattatatactaagaatgtttggtgattatctaggatatgaagtaaacttaagagaagtaaaatatattgtagagaaatataatttaattgataaaataccattgacaaaagagggtatgttggattataataaacttatacatttagtagaggaagaggttaataactatgagtaagaagataaaggagcttatccttcataaatcaatgaaggatatacattttgcaagagaagtactagataacttacctaagaacctattttcagcagaatctgaagatatgggttacttatttacagctataaaaagaacagcacatatttccgataagatgtctaatgaagcattagcaattaaagtagaacagcttatgggaaataataaggaagatgaagaaaaagtaaccaagacattgacttacttagaagatttatataaagtagatgttaatgaaaaagatgaatctgttaattatgaaatagagaaatatattaaaacagaaatgtctaaggaagtattagttaagtttattgcagaaaataaacaagaagactcagacaatttacatgaacttgtagataaattaaaacaaatagaagtaagtgatatttcaggaggtaacggagaatttattgatttcttcgaagacactgaaaagaaacaagaattattaagtaatttagctacaaataaattctctacaggatttaactctattgataatcatattgaaggtggtatagcaagaggagaggttggattaattatagctccaactggtagaggtaaatcattaatggcttctaacttagctaaaaattatgtgaagagtggattaagtgttttatatattgcattagaggaaaaaatggatagaatggtattacgtgcagagcaacaaatggcaggagcagaaaaaagccaaattgtaaaccaagacatgtctttaaacagtaaagtttatgatgcaatacaaaaccattatcagaagaatagaaagttactaggtgacttttatatttctaaacatatgccaggagaagttacaccaaaccaactagagcaaattattgttaatacaacaatcaagaaagataagcatattgatgttgttattattgactaccctcacttgatgagaaatccatatgctaaatatcactcagaatcagatgcaggaggaaaactgtttgaagatattcgtagattatcacagcaatatgggtttgtatgttggactttagctcagaccaaccgtggagcttatggttcagatgttattacaagtgagcatgtagaaggttctcgtaagattgtcaatgctgttgaggtttctctagcagtaaaccaaaaagatgaagaattcaagagtggtttcctaagattatacttagataaaattcgtaacagttctaatacaggggaacgatttgttaatcttaaggtagaaccaactaagatgattgtaagagatgaaacaccggaagaaaaacaagaacatatacagctattatctgacaatggaaaagaagatacaagtaaattccaaaataaagataataaaatagaagctataaataacacattcggaggattaccgggagtttaaatttttactatataccacttgacattttatatgttaggtggtataattattttataaagaataaaaggagagattaatatgaaaattaaacaaatattacctagtaataatccaaacaaatatcaccttttgattgagagcaagctaatacctagttactactcaacaagagatgatgtttataatgattattatgatttaaatctatatggtatgtttgatgatagtgaaaatttattatgttcatgtgcaatagaaaagtataatgaattagtatttatgaaaaggtttgttatggttggggaacatggtaatggatattttaaacagatggtttctcttataataaaagaagaacctcatatatgtttaactataaataaagataatattaaagtagctgatatcttaaatgaactaggatttattaatgcaggagaagtaattgatagaacaggaaagtatacttatgaattataccaaggaggaagagcttaatgaagtttgtattttttacagatagtcattttcatttatttacaaactatgcaaaacctgatgatgaatttgtaaatgatagatttaaagagcaaatagaagcattacagaaagtttttgatattgctaggaaagaaaaggctaatgttattttcggaggagatttattccacaaacgtaattcagtagatactagagtatataacaaagtatttagtacatttgctaaaaataaagatgtccctgtattattacttcgaggtaatcatgatgctacaactaattcattatataccgattcaagtatagatacatttgagtatttatctaatgtaagtgtaataaagtcattaaatacaattttaaaagataatgttaatattgtatttacagcttatggagatgaaacagaagaaataaaaacctacattaacagtaactatgataaagatatggttaatatactagtaggtcatctaggtgtagaagggtcattaactggtaaaggttcccatagattagaaggagcctttggatatcaggacttattacctgataaatatgattttattttactaggtcattatcatcgtagacaatatttccaaaacccaaaccatttttatgggggttcattaatgcaacaatcattttctgatgaacaagaagctaacggtgttcacctaatagatacagaaaagatgaccacagaatttattccaattcatacacgtagatttattactatccagggagaagatatacctgaaaactttgaacaattaatagaggaagataattttattagagttattggtacctcaaaccatgctaaagttttagagatggatgatagtatgaaggataaaaacgttgaagttcaaattaaaaaagaatatactgtagagaaacgtatagatagtgatgtatctgatgaccctctaacaattgctagcacttatgctaagcaatactcacctgaatcagaacaagaaatacttgaatgtttgaaggaggttttatagtgagaaaatataaagaatatctaaacaagtcagatgaagaaaatttagcagaagattgggaacggataaaagaagatttatggaaagtgtttaaagatatgaaacctaaaattaatacattagacatcagtaatgtagtaagtaaagacttagataaaagtaagcctattttacaatttcaagattcagacagagtaatagagaacatttgtaatgttgaaggtttagaagatggtttatctaaaatgaaaaagatttttgacgatagtaattttgaaaaacattactataatagaattgtagaacatgatgaatattattggattgattatggttctcatcattgtttctttagggttacgaaaggagataagtaatggtcgtatttaaacaagtagaagttaataattttttagcaattaaagaggctacactagagttagataatagaggtttaatacttattgaaggtgaaaataaatccaatgagtcattccattcaaatggttccggtaagtcaactttaatatctgccattacttatgctttatatggtaaaactgaaaaaggattaaaagcagatgatgtagtaaataatattgagaagaaaaatacatctgttaaacttaagtttgatattggagaagatagctatttaattgaacgttatcgtaaagataaagagaataagaacaaagtaaaattatttgttaatgataaagaaattacaggctcaacaaatgatgttactgataaacagatacaggatttatttggtattgagtttaatacgtatgttaatgctatcatgtatggtcaaggagatattcctatgttctctcaagcaacagataaagggaaaaaagaaattcttgaatctattaccaagacagacgtatataaacaagcacaagatgtagcaaaagagaaagttaaagaagtagaagaacaacagaataatctaagacacgaaatagataaactagggtatcagttatctacaaaagatgaatactttcaaaaggaaattgaacagtataatcaatataaagaacaattggctcaaatagaaaatagtaataaggaaaaagataaattaagggagcaagaggagaagcaaatagaagctcaaatagagcaattaacttcacagataccaacaatacctgaagatgaatttaagcactcagaggagtataataaagcttctcaaagcctagatttactttctaataaattaactgaactaaatcaagtatatgcagagtataatactaaagagcaggtactaaaatctgaaatagctacattaagtaatagcttaaatcaattagatacaaatgaccattgtccggtttgtggttctcctatagataattctcataaattaaaagagcaggaaaatattaataatcagattgagaataagaaacaagagattactagtgtactagaaattaaagatacgtataaagaagctattgataaagtaaataataaatcacaagaaattaaagacaaaatgtctcaagaagaccaacaagaacgagagcacaacactaggattaataatatcattcaagaggcttccaagattaaatcagatattagttcattagagaataataaaacttatttaaaagtgaaatatcaacatcaatctgttcaaggattagaaagagaagaaccaagtaaaaagaaacatgaagaagataaaaaagaattacaagaatctattgacaaacatgaagagaatgtagtacaattagaaactaagaaaggtaagtatcaacaagctgtagacgcttttagtaataaaggtatacgttcagtagtgttagactttattacacccttcttaaatgagaaagccaatgaatatcttcaaactttatcaggttcagatattgaaatagagtttcaaactcaagtgaagaatgctaaaggagaactaaaagataagtttgatgttattgttaaaaatagtaaaggtggaggctcatataaatcaaactcagcaggagaacaaaaacgtattgacttagcaattagttttgcaatccaggatttaattatgagtaaggatgagatatctacaaatattgcactttatgatgaatgttttgatgggttagacactatagggtgtgaaaatgtaattaagttattaaaggatagacttaatacagtaggaactatatttgtaattacccataatacagaacttaaaccactgtttgagcaaacaattaaaatagtaaaagaaaatggagtatcaaaactggaggaaaaataatgaaattaaagattttagatagagataatgcaacacttaatgtgtttcatcgtaataaggagcacaaaacgatagataatgtacctactgctaacttagttgattggtatcctttaagtaatgcttatgaatataagttaagtagaaatggagagtatttagaattaaaaaggctacgttctaccttaccttcttcttatggcttagaggataataaccaagatattattagagataataaccatagatgtaaaataggatattggtataatcctgcagtacgtaaagataatttaaagattattgagaaagctaaacaatatggtttaccggttataacagaagaatatgatgctaatattgtagaacaagggtttagagacattggagttatattccaaagtcttaaaactattgttgttactagatacctagaaggtaaaacagaagaagaattgagaatttttaacatgaaatcggaagagtcacaattaaatgaagcacttaaagagagtgatttttcggtagatttaacttatagtgacctagggcagatttataatatgttactattgatgaaaaaaattagtaaatagtaaggaaggatattatgaggtttgaagactttttaacccaagaattaggagaaccaaaagaaaatactataggtgaactaagatactgttgtccgttttgcggagaaaaaagttataagttctatgttaagcaagctttagactctagtaatggtcagtatcactgtaaaaaatgtgatgaatcaggtaatcctattacatttatgaagacttattataacattacaggtaaacaagcttttgatttattagagtctaagaatatagatatagagagagcccctttacttacaactaataataaggatttaacagaatcagagaaacttatattaatgcttagaggcgtacaccaagataaaggaaacactaatcttaaacctcctagattacctgagggatataagttattaaaagataacttaaataataaagagattataccttttttaaaatacttaaaaggcagaggtataactttagaacaaataattaataataatataggttatgttattaatgggtgcttctataaagttgatggagaatcaaaagtctcattaaggaatagcattatattttttacgtatgataatgatggaaactaccagtactggaatacacgaagtatagagaagaacccttatattaaatctatcaatgcccctgctaaacaagatgaagtaggaagaaaagatgtcatatttaatttaaatatagcaagaaagaaaaaattcttagttataaccgagggtgtctttgatgctttaacctttcatgaatatggagtagcaacactaggtaaacaagtaacagaaaatcaaataaaaaaaataattgattatgttagcatagatacatcaatatatattatgttagatactgatgcgttagataataatatagatttagcttataagctaaaaacgcactttaacaaagtttattttgtaccgcatggtgatgaagatgcaaatgatatgggaacaaggaaagcttttgagctattaaaacagaaccgagtactagtaacacctgaaagtatacagagttacaaaatacaacaaaaacttaaactttaggcttgaccttagagaagttttatgttatactaataattaagtaattaataaaggagaaaaaataatgtcaaataataaaaaagatattttagaatttgtagatgaatacattacagctttaagagttggtaatgagcaacgacaacatcaattagaagaaatgggtaaagaagaaacagcaacattaacagatgtagctaaagctattactaaccttatgttaggtgttaatgagcaaatgacagacttggaatacaacaatgagttaaatttaaatattttaattgacgctttatataaagcagaacttattaatgaagatgtactagattacatccaagaatcaattgataaatcacaagaagaacctaaaaatgaagaagaaaaaggagaacaagaataatggaaaaaaatattagcacacacacaaaaggtattagtcaagcagacatggagaaatggattgaagctgtagtccaaggaactgttgatggtaaacaagttgatgaaaaaacagctaaacaactagatagaattggttcacgcagtgtttctttagaagaagcaactcgtattgctaaagtcattaatgctgttacagttcaagagatgtcaggagattttaatgacgcatttaacgcaattgacttaatgatgattgttatggaagatgagttaggagtaactcaagaacaagttggaaaagctaaagataagttaaatgagaaacgagaagcttatttaaaagagaagcaagaagaattacgtcaaaaacaacaagaagagacacaaaaagaaactgaatctgacagcaatgaaaaagtaattcagttgaagaaaaatgacgaacagtaagaaaaaaggagatacattcgaacgtaaaatagctaaagaattaactgcttggtggggataccaatttaataggtctcctcaatcaggtggtgcctcttggggtaaagataataatgctgtcggagacatagtagtacctcaggaagctaattttcctttagtagtagaatgtaaacatagagaagaatggactatagataacgttcttttaaacaatagagaaccacatacatggtgggaacaagtaattaatgatagtagcaaggtgaataagacaccttgcttaatatttactagaaacagagcccagagttacgttgctttaccttatgatgagaaagtatatgaggacttaagaaataatgaatatcctgttatgagaacagattttattattgataatattagaaaagataaatttttttatgatgtccttataactaccatgaatgggttgacctcatttacaccttcttatattatatcttgctacgacaaaaaagatataaaaccatacaagaaggttgagtctaatttatctgaggtaagtaagcatgaagatgaattgattaatgaccttcttaatgatatataaggaagggtaagataggtatgacaagtaaagaaagaccattaattgtatatttttcaggtacagggcagacagaaagactagtaaacaaaattaatattaataactcatttgaaacgtttagggttaagagtggaaaagagaaaataaataaaccttttatactaataacaccgacttatatgaagggtgcaatacctaaacaaatagaaagattcctagaaattaatgggagccctaaggaagtcattggtacaggaaataaacaatggggctctaatttctgtggagcaagtaaaaagatttcagagatgtttaagattcctttaattgctaaagtagagcaatcaggacactttaacgagatacaaccaatattagaacactttagtaataaatataaagtagcgtaaaggatgagagatatatggcaacatatggaaaatggattgagttaaataatgaaataactcaattagatgacaatggaaaaaataaactctataaagaccaagaagctttagatgagtatttaaaatatattgaagataatacaagaaagtttaataatgaagtagaaagaattagagtattgacaaaagaaggaacatatgataaaatatttgataaagttcctgatactattattgatgaaatgactaagctagcttacagttttaattttaaatttcctagttttatggcagggcaaaagttttatgagtcttacgcatcaaaacagtatgacgagaataaaaaacctatttttgttgaagactatgagcaacataacgttcgagtagctttatatttatttcaaaatgattatgtaaaggctagagagttattagtacaacttatggagcaaacatttcaaccatctacacctacgtataataactcagggcaagctaatagaggtgaactaagttcatgttatctatttgtagtagatgattcaattgagtccttaaattttgttgaggatagtgtagctaatgctagttctaatggtggcggagttgcaattgatttaactagaattagaccgaaaggtgctccagtacgtaatagacctaattcaagtaaaggtgttattgcttttgctaaagctattgaacataaagtaagtatatatgaccaaggcggggttcgacaaggtagtggtgcagtctaccttaatatattccataatgatattttggatttattaagttctaagaaaataaatgctagtgaatctgttagacttgataaactatctataggtgttacaatacctaataagtttatggaattagttaaagaaggtaaacctttctacacgtttgacacttacgatattaacaaagtatatggtaaatatttagatgaattaaacattgatgagtggtacgaagaattattaaataatgataaaattggtaaagtaaaacatgatgctagagaagtcatgacagatattgctaaaacacaattagaatcaggttacccttatgtattttatattgataatgctaatgacaatcaccctcttaaaaatctaggtaaagttaaaatgagtaacttatgtacagaaatttcacaattacaagaggtatcagaaatttatccatattcctatagtaatcagaatgttattaatagagatgttgtttgtacattaggttcacttaacttagttaatgtggttgaaaaaggtttattaaatgagtccgtagatattggtacaagagcactaacaaaagttactgatattatggatttaccatatctacctagtgttcaaaaagcaaatgatgacattagagctatcggtttaggttcaatgaatttacatggacttttagctaagaatatgattagttatggttctagggaagctctagacctagtaaacagtttatatagtgctattaacttccagtctattaagacatctatgttaatggctaaagaaacaggaaaaccatttaaagggtttgaaaagtctgattatgctacaggtgaatactttgtaagatatattagagagtctaaccaacctaaaacagataaagctaagaaagtcctaagcaaggtctatattccaacacaagatgattgggatgaattagctaaagcagttaaagtatatggactgtataatgggtatagaaaagcagaagcacctactcaatctatatcatatgtacagaatgctacaagctcaattatgcctgtacctagtgctatagagaacagacaatatggagacatggaaacatactatccaatgccttatttaagtcctataactcagtttttctatgaaggagaaacagcttataagattgataataaacgtattattaatacaagtgcagtagtgcaaaaacatacagaccaagcagtatctactatactttatgtagaatcagaaataccgactaataagttagtatcattatattactatgcttgggaacaaggattaaaatcattatattatacacgttcacgtaaactttctgttattgaatgtgaaacatgttcggtttagaaaggaaatagatatggatattacacaaaaagtaaaacaacataataaaaatgcagtattaaaggcaacaaattggaatattgaagatgacgggatgtctgatatttattgggagcaaggaatctcccaattttggactcctgaagagtttgatgtatcaagagatttaagttcttggaacagtttaactgagagtgaaaagaacacttataagaaagtccttgcagggctcacagggctcgatacgaagcaaggaggggaaggtatgaacttagtatcttatcatgaaccaagacccaaataccaagctgtatttgcgtttatgggtggtatggaagagatacatgctaaatcttatagtcatatctttacaacactattaagtaataaagaaacaagttatttactagatacttgggttgaagaaaatgactttttaaaagtaaaagctcagtttattggatattattatgaccaattattaaaacctaatcctactgtatttgatagatatatggctaaagtagctagtgccttcttagaaagtgcattattctactcaggattttattacccactacttcttgcaggaagagggcagatgacacaatcaggagctattatttataaaattacccaagatgaagcttatcacggttcagcagtaggattaacagctcaatatgattacaatcttttaacagaagaagaaaaaaaattagcagatagagaaacttatgaattattaaatattctttatactaatgaagtagcgtatacacatagtctatatgacccgctagagttaagtgaagatgtaattaactacgttcagtataactttaatagagctcttcaaaaccttggaagagaagactattttaaccctgaaccttataaccctattgtagaaaatcaaactaatgtagacagattaagaaatgttgatttctttagcggtaaagcagactacgaaaaatctacaaacattaaagatattaaagatgaagatttctcattcttagatagtaaagagtatagtactgctaaggaattcctataaaaaggagaaaagatattatggatagaaaagaagcaatggatttattaagtaaaacagaaatattatttaaaaaacatgatgagttttcatgtgtgagtgatatcaatgaccctatgaagttatttagtaactctaaagatgccaaagctgatgatacgtctaagtcttttcagttagagtttatgcatgatatgactatgtatactttatcttatggctcaggacagttaaaactcattgatttatcagaaggttatgaagcacaaaaagctacagcagttaactcatttcccgaaattattaaaacattagaaaaggatgactcagaagatggaaaaaatgaataatttagtagatttaaatacagcaattagacaaaagaaagatgttattgtcatgattacacaagataattgtggtaagtgtgagattttaaaaagtgtaatccctatgtttcaagagtcaggtgacattaaaaaacctatcttaacattaaatctagatgctgaagatgtagatagagaaaaagctgttaagttattcgatatcatgagtacaccagtattaattgggtataaagatggtcagttagttaaaaagtatgaagaccaagttacacctatgcaattacaagaattagagtcactttaatttggaatttcctactatctgtgctatactataatagtacaaggtagtaggattttttaatggaaggaagatgacatatcgcaaagaataaaacattaacgatatataatagtgatagatattttaatatacacacaaaagataaagataaaattaatgaggctattaaagtcacacatggtaatgaagaagaaattgaaaagaatatggatgaattaatatctaagtctagacgatatatcatgagagatgaaaatcattatatgttatttaatgagaagtacaataatgatagacttatagaaaaagtatgtaaacacggtggcaaagttacatactatactgattcagtattaccctattatgttttaaaagacttatctagtcaccctgactcagaagttgtttatcgtatgcgcaatggttttactgcaaaagaagtagataatatagctttatcattcatgggtacaaaagttattattgatatttctgtagtatttccttatgtaaacccttatgatattattagaagtttacatgatattaaaacaaatgtagatgaagttcatttatcatttccacgaatattagaggtagatgaaaaacaagaaaagttttatttctttgatggtgaagcttatgatttaaaacctgaatataaagtcgattttgcagataaaattagagtatctttatcagtatggaaaatgtatatctatatcttaacaagtagtcgtgattttgaggatgtagacaatgtaattacgaaactaaaacaacaacgaaagattaagatataaggtgattatatgagtacagcaaatagaagagacatagcaaggaagatatcagagaacacaggttattatatacaagatgtagaagagatactaagtgcagagacagatgctatttctgacttactagaagaagggtatactaaagtaaagaatcataaatttatgcaaatagaagttattgaaagaaaaggtaaaaaagcttgggatgggctgaataaagaatatttccatttacctaatagaaaagctataaaatttaaaccacttaaagaattagaagaggttattgatagacttaatgaagaagagaaataattctcttctttttttattgacaagatttaaaatatatggtatagtattattaagttaaaaaaggagaggaattaaatgaaagtattaatcttatttgaccacattagagaagaacatttttctgtaagtaaagatgggagtgtgaaatctaatgtattaaatacacctaatggaaaaacacttaagaaattacttgagaagtgttctaacttaaagagagataaaacaaacagagactatgatattgattttctctataacgcagtacctacacctattagaaatgattatggtaaaattattaaataccaagatgttaaacaagcagaagtaaaaccatattatgaaagaatgaataacattattattgataactcctatgatatgataattcctgttggtaaactaggtgttaaatacttattaaatgttacagctataggtaaagtaagaggagtaccaagtaaagtaacaattgaaaatgaaatatcttctcatgatgtatgggtactacctacgtatagtattgaatatacgaatgtaaataaaaatagtgaacgtcatgttgtatcagatttacaaacagtaggaaaatttgtagaacaaggtgaagaagcatttaaacctaaagaagtatcttacgaattagtagataacattgaaagagtaagggaaatatttaataaggaagtaaagaatgataattatgatggtgtagatatcaccgcatgggacttagagactaactcattaaagcctgataaggaaggaagtaaacctttagtattatcactatcatggagaaatggtcaaggtgtgactatacctttatacaaatcggactttaattgggaaaatggtcaagaagatattgatgaagtactagaattacttaagaattggttagctagtaaagaagatattaaggtggcacataatggtaaatatgatattaaatttttaatgagtactgagaactttaaagattttgagaacattcaagatactaaagtaggttggtatttagctgtcacacaagaagttaaagaatctttgagattatctgacttagcttacgaggtaacagatgtcggaggttatgataaaccactagaagattttaaattatggtttgttactaagttattaagatttttctctgataaaattaaagaaatacagaaagaaaataaaaaaattgctaagaaagagtatgatgttaaagctcctgaatacaaagagtggttagagactaaattaaatgaaacaatagtagaattagatgatactgagaagaaatttagagttagtgaattagagaaaaaatacattcagttaggtctttcacctgaaattgtaaacatgaatttagttatgaacaacgatgaatttattagtattgcagagcaatcacctgaatatatgggattatctgattacgctaaatcttacacactgaatactgcaattgatttaattaacaagtatagagatgtaaaagatgtaattaatgatattgatggaggtaactttaactatgattggttccctattgagttaatgcacccgtacgcatcaggagacactgatgtatgtagaagaattcattgtgatgttgttaagaaacttaaagagcaagatagacctaagtcaatgcatttattagaagttaattacccaagacttactaaatcattagctagaattgaatcaaatggtttatactgtgatttagattacatgaaagaaaatgatgagtcatacgaatctgagatggctaagaaccacgctactatgagagaacattgggctgttaaagaatttgaagagtatcaatataacctttaccaaatggcgttagaagagcatgagaaaaagccaaaagatagagacaaagagattcatcagtatagagataagtttaaagatggtaaatggatgttttctcctagctcaggagaccataaaggtagagtaatttatgatattttaggaattcagttaccttatgataaagagtatgttaaagaaaaaccatttaacgctaatgttaaagaagcagaccttacttggcaggattataaaacagataagaaagctattggttatgcattagataacttagaattaaaagatgatgttaaagaacttcttgaattacttaaatatcatgctagtatgcagacaaaacgtaactcatttactaagaaattacctaatatgattaataaacaaaaacgaacattacatggttctttttctgagacaggtacagagacatcaagattaagtagtagtaatcctaacttacaaaacttacctgctcatacatcagatgtaaacaagtttgattataaacatccaattaaacgttcatttgtttctagatttgaaaatggagtactactaggagccgactatagtgccctagagatgcgtattattggattatttactaaagaccctgatatgctacaatcattcttaaatggtgaggatattcataaggctacggcaagtattgtttataataaaccagtagaagaagtaactaaagaagagagacaagcaactaaagcagttaactttgggctagcctttggagaatcaccattctcatttgcaggtaaaaataatatggaagtaagtgaagcagaagaaatatttgaaaagtacttccaaacaaaacctagtgtaaaaacttctattgacaatgtacatgaatttgtgcaacaatatggttatgttgatacaatgcacggtcatagaagatttatacgttcagcacaatcaacagataaaaagataaaaaatgaaggtctaagacaatcatttaatactattatccagggttcaggtagtttcctaacaaatatgtctttaacttacttagatgattttattcagtctcgtaacttgaaatcaaaagttattgctacagtacacgatagtatcttaattgattgccctcctgaagaggctaagattatggctaaagtgacaattcatattatggagaatttaccgtttgatttcttaaaagcagaaattgacgggaaagaagtacaatacccaatagaagctgatatggaaattggtttaaattataatgatatggttgaatacgatgaagaagaaattgatacatttaattcttaccaaggttatattaagtacatgatgaatttacagactttagaagattataaagagtcaggtaaattaactgatgaacaatttgaaaaagctactaatgttgttaaaagtgaaaaacatatctatcaagaaatttaataaaagtattgacaatatgtttcacttatgttatactatataggtaataagtataaggaggaaaaaagagtgaatacaggagagattagatttaatcgttctatggatgaatggattataacaagtatgtaccaggatgaattaggtgaaatgaatattgttgttacattctataatagagaagaaaataaacatggctctaccgttttaccaacagagtcatctactggagaagtaacagaagaattagcaagtcttgaagaagaatatcctttagctttaccgttaagtagtatttcagttaatatttaaaaggaggaactgataaatggagatacacattgattccctagattttacaaattttactattaaagatagaaatgggaactcacaagagtttgatattacagatgaattaaagattacagaatatacaatacaagaagattttatgcaacaatcagctaagtacgctttttgggcttctatattagagaaggtcagagcatattctgaaatggagcaaagaaatttagaaacaattggtagtaagcttaatctcacaattagacaagagtatgaacaacaaggtaaaaagcctactaaagatatgattgagtctagtgtgtatattcatgattcctatcaacaacaacttaaagttgttgaggcttggaattataaagttaaacaacttcaatatgttgtaaaagcttttgaaacaagaagagatatgatgattcaattaggtgcagagttacgacaaacaaataaaaatggtggaattactaacccattttcacattaaaaaagaaagtaaagaatataattgacaaatataaaaaactatgttataataaataagtaaattaattaaaaggagaaaagataattatggatttcaatcaatttattaacaatgaggcaagcaaattagaaagtaataacagttcttttaacaacaatgtagagagctataaacctaaaaaccctgtattacgattaggtaatattaaagatgcaaatggaaataaagttactaaagaaaatgcttttgtacgagtattacctcctgcacaaggaacaaatgttttctttaaagaatttagaacaacaggtattaactactctaagaaagatggttctcaaggattcacaggactaacattacctgcagaaccaggttcatctgtccttgacccttacattcaggactggataacaaatggcgttcaatttagtagattccctaataaacctggagtacgttattacattcatgttattgaatactttaataataacggacaaattcaacctaaaacagatgagcaaggaaatgtaatgattcaacctatggagttatcaaacacaggatataaagaattggtagcaaaccttaaggatactatgttaaaaccgtcacctaatgcacctcatagttttatttcagcaaatgaagcattcttagttaatattgctaaagctaaaaaaggtgaaatgtcatggaaagtaagtgtttatcctaatgctcctttaggtgcattacctcaaggatgggaacaacaattatcagacttagaacaattagcaaaaccaacagaagaacaaaaccctaactttgttaacttcttaatcaataacgttaataacacagagttaagtcatgataactttaaatttaaccgtgagtctaatgtattaggtgaagaaccttcagaacctaaacaagctcctactcaacaagatattgatagtcagatgccaagtaatattggaggtcaacctaaccaaccacaacaaggtcaagtaggtcagtatactcaacaaggtcaaaataatggtcaaggacagcagttacaaggtaaccaacagccaattagtaatactcaatttggtcaaggaacaccttcaggacaacaacctaataatacaggttcagttgattgggataacttagctcaacaacaatcacaaccggattcaaatccttttaatgactttgatgtaaacagtgttgatgattcacaagtaccttttgagacacaacctcaaaacacacaacaagcacctgaaccacaacaaactactcaggagcctccaaaacaaaaacaaacacaaagtattgacgatgtattaggtggtctagacttagataacctataagatatagagtgccttagagcactcttttatttgagatataattactaggaggatattaaatggcaagagcaaaaaaaggtaaagaagtagatttaacagatttaaatacaattgatttaggtaaagaattaggattaacattattatcagatacaaatagagcagatattaagaatgttatacctacaatggtacctcagtatgactatattttaggtggaggtataccgttaggtagattaacagaggtttatggtttaactggtagtggtaaatcaacatttgcagttcatttgtctaggattgcaacacaattaggtgttattaccatttggattgatattgaaggaacagcagacaataatcgtatggaacaacttggagtagatgtttcaaaattattctctattcaatcaggagaaggtagacttaaaaatacagtagaattatctgtagaggctgtaggtaaagaattagagtactggattgacacatttaatgaaaagatacctggagtacctattgtgtttatttgggactcactaggagctacacgaactcagaaagagattgacggcggtattgatgagaaacaaatgggtcttaaagcatcagctactcaaaaagtaattaatgcagtaacacctaaattaaatgatacaaatacagggttaattgttattaatcaagcacgtgacgatatgaatgcaggtatgtatggtgaccctattaagtctacaggtggtagagcatttgaacatagtgctagtttacgtattaaggttcataaagcatcacaattaaaacaaaaaagtgaattaactggtaaagatgaatatcatggtcatatcatgcgtattgaaactaagaaatctaaattatcgagaccgggacaaaaagcagaagcagacttactatctgattatatggtaggtaaagaagatgaccctatattattaaatggtattgacttagaacatactgtatataaagaagcagttgaaagaggtttaattactaaaggtgcatggagaaactatgttacattgaatggtgaagagattaaacttagagatgctgaatgggttcctgtattaaaagataatagagagttatatttagaattattcagtagagtatatggagagcatttccctaacggttactcaccattgcttaataataaagtaattgtaactcaactagaagagtatcaagctcttgaaaattactataaagaatgggctacagataataaacaaaaggaacaagaggaagaactaaaaggagaatctcaagaaaaggattctgaataataaatggataatttaatagataaaaacatgaatcaggtaaaagaatctttggggaatgcaaattcctcagatgttcttcctttaccttataaagatatagcaaagaaatttgaagaagtaaaagaaaaaggtgaatcaattatcattgaagaaggtggattcccttacacagattctacagtgatgtatatagaacatgtaacagatagatgggcaggaggatactccttaattagacatgaaggtgaagaggttaaagtgcctaaaactatccatttctctgatatatatgttaaggataagtcacataaagtaagaataatcttcgagggggctaatccttatgaagaaagctaacaatggtaatagatatgtaatagatatagatggtattcctgttgattttgaaagagacttggatagtttacttaacaggtataaaaaccttagatggtcgttgtatcatagatatgcaggtatattatctaatgactttgaaagacaagaattaagagaatatattgatgagcaatttattaaactagttaaagaatataatattagaagtaaagtagattttccagggtatattaaagctaaattaactttgagagttcaaaatagttatgttaaaaagaatgaaaagtataaacgtactgaaattatcggtaaaaaagattatacggtagagtccttaacagaagatttaaatgaggactttgaggataatcaaattatgagttatgtatttgatgatatagaatttacagaagttcaaagtgagctacttaaagaattacttattaatcctgaaagagaagatgatgcctttatagtttctcaagtagcggagaagtttgatatgaaaagaaaagaagtagcaagtgaattgacagaattaagagattatgttagatttaaaataaatgcatatcatgagtactacgcaaagaaagaattaaataatcatagagttaatactgaaaatcatatttgggaaaactagttacagtgccttccttgtgttatattattatcgagaattcaataataaagcatagggaaggcttttttctatgtcttatagaatgctttaaaatagattactaaaataaagattggagattaagcttatggctaaaaagaatgttaatgatgtattacaacaagaatctgttacagtagcagataagtatttacaagttaaagttaaccgtgacggttatactcgtacacacgaaggacaatatgcgtacaaagtagtttcagagggagaagagttattcttatacccagtacaaacagatggtaaaggtacattaaatgtaatgaagaaatcacctattgcttacactgatggagacaatatccattttgtagtaaatacagtagtagacccttataatcactcatttatccgtactgaagatattaaaggattagataaaggtaaacaacttattcaagctttcttagctttcgttgaagaccgtttcaaatttggtgtttataacgtatttgttgcaaataacaaagaagatgtattatctatcgtagaccctgcagataatgatgcagatgaagttaaagatagtttagagcatgcacatgaagatgtaattgcggatttccctgctagccctgctcgtaaggacgttaaaggcgtagattcaggagaaggtcaaggagacacttcagaaccatcagcacctaagaacgttcaagttactcctaaggaagacggagcagacgtatcagcagaataatatagataaaggatggtaaatttggctaagttaaatttatacaaaggtaatgagttactaaacagcgtagagaaaacagaaggaaaatcaacaatcacgattgagaatttagatgctaacacagattaccctaaaggtacttttaaagtatcattctcaaatgattcaggagaatcagagaaggtcgatgtccctcagtttaagacaaaagcaattaaagttatttcagttacccttgacgttgatagtttagaccttacagttggagatactcaccaactatcaacaactatcacgcctagtgaagcatctaacaaaaatgtgtcatttgaatcagacaaatcaggtgttgctagtgtaacatcagaaggattaattgaagcagttagtgcaggaacagctaatattactgtaactactgaggatggtagtcatactgatattgttgcggtaacagttaaggaacctattcctgaagcacctacagatgtaacagttgaacctggtgaaaatagcgcagatattactgcataggaggacaataaagaatggaaaagacattaaaagtttatagtaatggtgaagttgtaggctctcaagtagctaataacgatggagctactacagtatctattacaggcttagaagccggaaaaacttatgctaagggagcttttaaagtagcatttgctaatgattcaggtgaatcagaaaaagtagatgttcctgaatttacaactaaaactcctgctgaagaaccttcagaagaagcataataattaagaccaactaaaaagttggtctttttttattgacaatttataatatctatgatacactatataagaattaagaaaaggagggaaagtaatggatattccaacaatattatttagaaatccatatgattacacaaaagtaaaaaaattaatggaaaacaaagaacagtacattgtagtaaaatttgattcagtttctgttcataatttaaatgttcaaggtatgatgaatgttattcaagattacctgcacatctatggttacagagtcaaagaatacggacaagaaaatgcttctaaagatgatgaaagagacgttaaaggttacttatacgaaagagtaggtgagtaaggtatgggtgttataataaattctaaccatattcagtcagatactttatatgagtacgatagtttttttgatattgagaaagtagatacgtttgaagaaggattactatcaatacaggatgaaccaactgttttagcaggatttatctatgatgatattacatttaataaggttattaactctaattcaaatattgataacaatattaagaataatgatatttattatgtttctgatatagggttacttccggatacttttatcactgttgattctaataaaaaatactattcattattacagcagatagttgagttaagtaaagaccctttccctaaatgggtagaaacggatgcaaaagatttaactaggtattataactttcaagattttgaagatgtattcgatttaaatagtttttataaaaaagaagttgacatggtaagagaaaagtgctataataatggtaatgtatatttattatatgaagtcttgcctgattataaattacctctagcttatagtttactttcaaataaagagcatggtattgttattatcggttcacagacacgttctaataatgatatactgactttttacgttaaaggtatggatgctaaggcaatagctagcatgtttaatgtagaacatgattatgattctaatatttttcatacattcgtaaatagtcacattaatattttaggaaatcaaataactaagtttataagagagaaaggaagcagttatgagtaactataaaacaatagaagaagtacaagcagttattatcggagtattatttaaagatgaaggtaaaattgtaacatctaagtttaataaaattactaaagagtttggtttagatagaattggtaaagacgaccttaaagaaattgttgaagatattagacaagatgcctatctaaatgagcttaagaataaagcaattaaaggtaaggtaactttaggagaccttaaagatgttgcagataatcaagtattcgaaggtaataactatcatgaagaagtctctacttatgtagtagctaaagaaaaagaactatctcacttgagagaacaacgtaagcacaacagacataccgcataccctcaaattatgtttgatgaacttaaagaacatatggttaaagaattacaaggggaaacattagtagagcatcatggaagtaaagttaacattaatgatacagagttaattgtattactatcagatttccatattggaagtattgtatccgatatgactaatggtaaatatgattttgaagtattgaagtcaagactaaatcactttattgatacgacagttaaagagattgaggatagagagatttctaatgtaactgtttactttgttggagacttagtagaacatattaatatgagagacgttaaccaggcatttgaaacagagtttactttagcagaacaaatctctaaaggtactcgattacttattgatatcttaaatgtactatctaatgtagttacaggagaattaagatttggtattattggtggtaatcatgaccgtatgcaaggtaacaaaaatcagaagatttataatgataatattgcttatgtagtactagactctttattgctattccaagagcagggattattaaacggtatagatattattgataatcgtgaagatatttatactattagggataatttcggaggtaaatctattatcgttaaccacggtgatggattaaaaggtaaaggcaatcatattaataaatttatcttagatagccatattgatttactaattacaggtcacgtacaccatttctcagtaaaacaagaagattttaatagaatgcatattgtagcttcatctccgatgggatataataattatgctaaagagctacatttatcaaaaactaaaccttcacagcaattattatttgtaaataaagaaaataaagatattgatattaaaacagtatttttagattaaggatggttaataaatggatacaatttttattataggtgtagcgtttataacttttgcaacatttaatatagtctttagattatttgatttatggactacagagaagaaaatggtaagtcaaggacaacctccactaagtaattttgagtactatcatgtgatagtaccctacttagttggtgttattgttattatactgagtattatttttagagattccttgtattctgcgcaatcaggattcggtgttattattacaagctttatttatatgctagtttatgttataatcggtcttgtagggtcgtttgtacttacaatattccaagctagaaaggctagacagtatcaaacacaggaggataataatgaagttcaatgatatttatgagcaattaattaaaaatgatacaatacaaaacattcatgaatctcaagatgatgaaggaaatatttatacaatacaatttgataaaggtaatgataagtatttatttaatgttatcaatgatggattcttgaaagaaatgacaaatggtatggtagaccatcctaaaggtcaaccatattcagtaagtctaatcaataaagaaactcctagtatgtcagtgaaacaatatttaacagacgtagaagatattgtacctactattagaaaaatggaaaaggatttcttatagagtcaagtctttacttgactctttttactatatatggtatattaatatagaggtgacttaaaaatggattttaattttagtgcttttgataatagctcattagcaatgagaattagtgagggtgtatactatttcaatgataccccttattactttattgaacatatagaagaagaaatgtctgagtatgttattgtgtatgacatacatgatagagaggaaaaagaaaatcctcagaagaaatatagagtagaaccttaccaacggacaatacctggaggtacacctcttagtaatttgattaagagtatgatgcctcaacgtaaatatcctaagaaggttgtagaagaccctatatttgtagctaatgttattcctttaggaacagatacagttacaggtaaaactggaaaaggtttttttgaaagagataaggatagaactatttactcacagaaagaaccaactaaggtagtccatggtcaatacacaggtgttttcataggtctaacaagtgttaaatggaatagaacatatactcctttagaaagtgttgttgagtattgcaagagagtaaaaggagataggttaaatgtctaatgatgtagttaagttttatgaaaaagatattaaagaccttataagaactaaaaaacacatgtttaaagacgatgagataactagtgatataaatgatatacgtatatttaatgagaaagtaatttgtcaaggtaagtgtagaacagattgcttagtactagaccgtaatggtactgtcatgggtatagaaataaaaacggaaagagattctacacagagattaaataaccaattaaaatattatagtctagtatgtaagtatgtatatgtaatgtgccatgataaacatgtacctaaagtagagcagatacttaaaagatataaacataatcatgtaggtataatgagttacattaattttaaaggtaaacctgttgtaggtaaatataaagatgctacaccatcaccacatagaagcccttatcatacaatgaatatactatggaagacaaatttaatgacaatacttaggttgattagagaccctcatacttatagaacagggtatagttataatgctagcggtaggtatagtggtggggaaggcaacttctctcaaacaactcaaagtaaaagaatgaaaaaacctgctattattaaccaaataattcattatgtaggggtagataatacttataaactctttacaagaggtgttatttatggttataataatagatgggaagttatagaagaagatttctttaatactatgaaaaatggggtaagagtaatcaatgagcaaagacaaaccaaatagacgtaaagagatacagcatcaacctgttaactttgccccaacaaatactttaacaggagctaataatagtttctttgctaaaaagccctcagaacctaaggatacaacatctgttattgaatatagaatactttttattaaaagatttgataatgtaacaagtacagatgttaagttacaaaaaaaatatgcattaaatcttattagtgaggcacttgatgttaaagaaacttatctatctcttaaacaaaaaggaaagaaaacagaatctattttacacacagatagagtttattatgtgcatagaggtaaaaaacttattggaaaatgtagtattagggagcaaagaacatttaaaggtaaacatttgatatttatatttaagacaagacatagagttaaagcagaaaggaaagataaataatgttaaaaggattttcagaacatgtagataaacctacaactattaaaaccttatataagaccttaacaagtggtaaagtagaattactaggtgtatcttatggtagtgattacttcccttcaggtgttacagtacaagcttatattgaagatataggtaatgaagatgaaggtctgcagtttgttaataaagtaaacgtagtggaatcaatgaaacaggctgtagtaggtatgaacaatcaattaggttcctcaggtcttggatatgttagaactgaacaacttaaaaaagagctagaagaaactggattaatgacagatttacttgctagaggtactaacttaacttctactaagaaagtggatattgtaagtacttttattgagcctgaagtaacatatcaagacattactatagctaaagatattaagttacgtctatacaaactagaagaagagtcaccattaaacggttatacacatattgtatatcttcttactacagagaaattatatgatggtcaaacactatttggtatgctatctaagaaagataagttatctaaaggagatactgataaattattagcattctttagaaataatagtttaataagtaaaagtgtattttgtgttaagttattaagtaaagactactacttcaatttatataatacacatgaaacaggtatattctttttagaagatacggatattattactattgcttgtggtcagtcatacgttaaagttaatactaaagatattaaatctagtcatgttaaaattgaagatgagactcataaattaactgagctagtaattaacttaaaaggtgacgacacattaactattttattctaatagaatgttataaatatgtgataattaagtataaatatacgttatatagaaagttttcataatgtttttaatacagaaactagttaagttttttacacttgtcctagtttctgtgaaattatatttatgaaaagttaaaatatcttttaggtaaaggctttgtaaatagttaaaaaatatattaaaattttatacaaagtagttaataaaattatattacatttatatattatgaaataataacagaaattgtgatatattatatagtgtaaccttgaaacagttgatgttgtagggtttgtttatgttcgttaaactggtttcagaacatcagttaccataaataaatgacagttaaggagagctatataatggctagaaaaaagaatttacgaaataaaaacagtgatataaaagttgttcctgataaagaaaaagaaagtatattatctaagctataccataataaattactacgctcaaaggtagataatgctttagatgaagatatgagttatgatgatattatagaactatgtaaagaatatgatttagaattgtctaaatcagctattacaagatacaaaagtaaaagaaaagaagctattgaaaatggttgggatttaggagaattaattgataaacgtaaaaaaacaagcgtaaaagatattaaggaaaaagaaactcctatattagaagaggagcaactttctccatttgaacaatcaaaacatcacacacaaacaatctatgatgatattcaagtactagatatgattatttctaaaggtgcaaaaggattagaatttgtggaaactttagaccctgctttaatgatacgtgcaatggaaactaaagataagattaccggaaaccaattaaaaggtatgtcatttattggacttagagaattacaattaaaacaaacagctcaagatacggctatgagtgaagtattattagagtttatacctgaagaaaaacatgaagaggtattacaacgattagaagaactacaaaatgaattctacaaaaatctagatttagatgaagaaagtagaaaattaaaagaagctcttgatagagtaggctatacaatttagatagtgaggttagagtaatggcagatgagattagtttaaatccaatacaagatgctaagccaattgatgatatagtagatatcatgacatacttaaaaaacggaaaagtactgagagttaaacaagacaaccaaggggatatccttgttagaatgagtccagggaaacataaatttactgaagtatctagagacttggataaagaatcattctactataaaagacattgggttctctataatgtatctgttaactctcttataacatttgatgtttacctagatgaagaatactcagaaacaactaaggttaagtatcctaaagatactattgtagaatatacaagagaagaccaagaaaaagatgttgctatgattaaagaaatacttacagataataatggtaattatttctatgcacttacaggagaaacaatgctctttgatgaaaataaattaaataaagttaaagattagggttgacagttctgaaagtatatgatatagtatatgtatactaaaaataaaggagctaacaattatgtttatttcattaaatcaagaagagaaagaattattaactaaagaggaaaacaagtatacaccattagaaacatcaagagagtttaacacacctaaagaagaattcattgtaacaagttataatgaaggtaaacctttagattacattgcaaaagaagctaaagtaagtatgggattaatttacacagtcttaaactactataaagtaggtaagcgtaacaagaaatcacctgtagaagaaagaattgcacatatcttgaaagacaaaaacttagttaaagagattattaaggattaccagtatatgaatttacaggacatttatagtaaatataatcttcataagaatggtttatattacatcttggatttataccatgtggaaagaaaatctgaacttaaggacaaagcattggaagaggataatattgtcgttgagtaagtaaagaggttataatatgagaaataaaaaatcatttcaagagcaattaaatgacatgcgtaataaagagaaatgggtatctgaagaggagttcactgaagaagtgactccttctgaagaacctgaagtagaagaagaaaagttatatactttaaatgagttaaaagaaaatttattggatgctcagggattaaaagatgttgtcgataattttcctgcatctaagccagtatatgaatctaataagctttatatttgtacaataccaaaaggatatcgttctacagaagtacaaccaggtcaatatattggtatcagtacagggttattatcagaatcagaagattttagtcatttaagaggtcaaatgcctagaaatctttatgaaacttctcatgttttaaaacctttagtacgtattaataatacaaatctcgaatatcaacagcatgagttacttgaagatattaaagatgacaagaagatatatgatgttgaattagaagacctgaggttagtaacaggagaagaaatatcccatttagaaattgtcgatagtaagttttttgaaagtcgtattaatgaaattctcgaccgctatactgaattaacggattccgatgatttgcttatatactatagtaaattacgagaattagtaggcagtgacaaaatgatttattgctcacttctagataaatgtgttaaaattatagattaatagttagtctcctcttatattataactgtaagaggagacattttttatagaggtgttaattatgtcaagaaaagcaagtatattctatatactagtagttattgttttggctttttctatttcatcttattatatatcttctttcatgtatcacgacaaagcaaagaatgaagtctctactgagttatcaaacacgggaaagattaaagaagaaaagaacgtagaatttgtcggtgactatacacttaaaaaagtggaaaataataaagcttactttatggaaacattacctacttacctacccggtagaacaggagataacagcatagatatgaggtactacaaaacaagtagatttaaagaaggggtaaatttcaagcttattagggtatatactgaagatggggaagataatccaattcataagtataggtttgaagcagtaccaaccaaaaagtaataaggaggtgacttaaatgacaacattaattgtcgtcatctttattgctatcatttattacttatggaacagtgattgagtcaagttaattcttgactctctttttgttttatggtatattaatatatagaaaggagagattaattatggaaatggcagatttagaaagatttgatacgtttgtaagattagtttcagacgatgagctttcggaggaaagaatattagaattgagtgtagacttattaaatccgatactagaaggaggtacagcttaccaagctaaaaaacgcattagaagtaagttcggtaaaatagaagcaaaaaactttaaaagaaattataaattcttactcaagtcgatagctcaaatagaccaagggagataggacaatgacagaaagggaaaagttagttaaagagattgaagatgctaatagggacatacaattaaggttaaaagaagtagatgattataaggatagtatacgttctaaaggaacaagaaactatgtatctactaaggtattagattcagttatggtagggctaattataagtttctttattcttgtaatgttacgtgtacttgaatattttgtaacaggtaatgctgtttattcacctttagcgcctgcagttattattatgtttgttttagccttaggtacatggaaagtaagtaaaatgaataaaatagtatcctacaggggaactattaaaatgtattgggaattaagtaatgctgagcagaaccaagctaagatatttaagtatcctaatgatgaagtagatatcgtatcaaaacataacttaagacaaataacttttagtgagattaatatccttcatcttaaatatatgagatataataaggcagtagaacagcatactaagttatctaaagaactttttaaaaaagataaagaaactattgacaagaacaaataagtgtagtatagtattactaaaggaggagagatattatggttatacctagtattaaagcacaaaataaattcaagaatgaattagagtattataagcaaggtcacattagtgaaagtaaaatgttagaattagcttttgattacattcaagaattagaacaaaataatgaatatgttactaatttgctagaagaggagagatatggtgagtaaatttatcggagtgtacttatttaatttattagtagtagcgctaatttatacagtaggatttttattcttttatggtgtagctagtttagttattattataactcatgccactattgacccatttgtattagctactttcttaggaataggattcttggttattagaactgcacacagaatcatggcacgagtaatcaatgatgcagtagcccaagccattaaggataaagaaaatgaataaaggggaatttattatggataaaacattaccaaagtttagtgtatatgaagttattgtaaagactgtaattatgacaccaacagaaggaagttctgaccgagaatcattttacttttcaactcaagagttagcagaaagatttgttaaagaaaaccaagtagaaacaaaagatggaaagcgtgtatctttttctattaaagagcgtaaagtaaatcaaccaggctaacattaatttgttagctttttttattgacaaatcattttatatagtgtatagtaatattatactgaaaaggaggaattattatgaaagtttcagaagaagtaaaacagagctacttagagaataaagctaatactaaaatggataagataagttggtctgagttaaggtctagtcctgcaggtagaacattaggagacttaatatactttagtgtagttataataaataatatcatagctatagcattaacattaatgcttggagatattatcaaagatttaacggatagtaccctagtaaaaataataagtatattacttattatcatcgttgtatacggtatactatcggctttaatacctatactaatatttaaatctgtatttcctggatgggaatatactgaatggaataattcttactatttgagattacctggagaaaaagattataaatactatagctactggtatttaaatttattaggtgttactgagttttatcggacaaatgatgagggtaaagaaattaaagaaactagtttaagttgggttttttatactaaggttgatagacctgaggatattgaccattggaaaaatcaattaataactaatagacctttaacaatttctgagtacaaaaaactaaagaagctagataaagaaagtgaaattagaaaacaagaagatttagaagaatacaagcaatacaatagtaattaaagaggtgaaaaaccaatgataagttcatttgatagtgtattacttgtaatatacattattatagcttttgcagtagctatggcaatactttatttagtatttaaaggtatgacccttttattagataaactaatgatgttattactaagtaaaactaccttagatgtagaagcttgctccatgataatggcagtagtcagtacaattgtgtttggaattattgtacttttaatatggttagcagtaaataatattttattgtaaggagttttactatggattttaatgactttataaacagtgaatcagatagagtgggtaaacctaaacaaaagaagaaggtagaaaataaactaccttcttctactcctattgaagagaaggaaaaaatgttgaaaagaataagagaaaaatcattatacattgatttaaggagaaaaagaaatgactaaagaaacaaatgtactttacaaagataagtatagagattatactatagttgtaagactagtaggtaatattattgttactgaagtagataagaagcataaaacaaaattttcacctattatatttgataatggtcgagaaggtgtagagcttgtaatgcgtatcggctcagaagaacttgatatgaaagaacttagagaatttactaaagaggtatcaacagctcagaaagctttagaatattttaataaaaaactttacattaaaggcttgacagatgaagccttttaatatatactaagattataactaaaacaaagaaaagaggaatgattattatgttattaggaattttatggtttatatggggatttgtatcttattttgtagtgatgtttggaattgaattttggaaagacagatggttaccaggagttattggtgcaggagttttattaatattcttattttggattatgaagtctattcataatgctatgacagtagtttacttgtattaggaggtaatatagatgaatatattagttatccattatgaagaaacaaataaacgagttctaaaagaaacaataaaaacaatacaaaatcatttaaacgatgaacatggtttggttaaaatgacagcaacaagacttagtagaaaagccatagaagaagagtttaatgattacaatatagttattgcagaaaatgaccctgatagtttttacagctactcagatgcagtagacgatgtagattttgttatagatataccaatttcatatttagatatacatgcaggtgtagagtgggatgttgataatcctgtagatatgctagataggaaccctgattttatagaagctatgggtcaattaaacgaggacttaatgttataaggaggaaataaaatgttaaatgaaaaactaaaaaatttagaagatacgaaagtgtatatgattaatagtattgcaagtttactaagtgcaagtacaggtaaatcaagtaaagtattttttgatgaaggaactattaaaattgtaagtggtgatacaaaagcggtagaagttattgataatttagttcaccctcattcaggacgtttacctattaaaacaacagaacgtattgctcttggtagactaacagattctttacagtttgttatttcagaaatagagtcggttaaagaccaaattatagatgaagaaaatgaagcttacattgattttgtaatggaagactggaattgggattaatgcctatggacttaatgacaatagcttctgttgcctttatagctgtcgttattattgatttaattaatgatgatatgagctatatgttaactggtactgcaatattaataaacatttgggcaggtttttatggatggttctttttactacaagcaggtatgctacttttcttactcttagctagaaaagttaaagatgataaggaatcaatactatactcaggtgcttcgttaatatgtgcattagggatgataataaatcttcttatattttcttaaaaataagtgttgacacttctaaacttttgtattatactataagtataacaagtacaggagatgattaatatgagtaaagaaacaattagaagacaattttcaaacgcaattgagattatggcaacaactaaggaatggtggaatttccctaaaagctttaatacaaataaagagtttaaaattaaaacttttaaaaatgacacacttgtatttgaagttcgagaaggtaatagaaacttaggaagctttgtagttttcacatgtattgactttgattatgataaacttgaaggaacttcaacacagtatatgattaattactttgctaagaaattaactaaagatatgtttaactatcataagttacaactatagtaggaggtggaaagatgagagaggaattaaaaccttttaatagaaaacaagttaatgttaaagggtacttagatgatgttaagtactcaaagcgtagaagacataaaggtaatcaacatggatgtgtaaaaataacagttactgacgtaaagattaatggtatatctattgaccatgttaatattgaagttggaatttctttttatgaaaaactaaaagaattacaaggaaaaagaattcaatttgtaggtactgtctacaaatacgttaaacatgctagaggtcataaaggtagaattaaaggattttataaagaggattatagtgtaactttggataagaagttacaaaaggaggaaaaataatgattaaaagaagaaaacatttagaccatacagtacaacctgagaaaggatggagaacagtaccttttaatggttactatgaagcccacccttcaggtttaattagaaataaagtaacaagaaaactaattaaaggaacacagacaaggaaaaatcatcctaagtggactgctcatgagattgtatatttaattaaccctaagaaaacaagttattcacgaggtgttgttattgcacatacctttcctgaaatgattagccaatcacgaggagaccttaagaacggtcacgtatgttttaaagatggtgacagaagtaattgtcatgtaaataatatgtttattggtaaaggtaatgttaataagaatatctacaaattaaatgattcctatttgactagaaaagatattgaagaggatatcaacaacttagttaatgagagactattctctcagttagagttacttattaagaaaaatgaacctgaaagaattacacctagcaatcactttattaaaagagataataatgtattcagtatcacggatttatctaaaaactctttagtagaatttgaattagaaatcaaaaatattaaataaggtggctatataaatgaatgagtggtatgctttatgttactatgataaagtaggtaaaaagaaaatacctaggcaagttagagcacacagagatatttcagtattagaagagctaaaagaaagattagaagaaagaaatcctagtacagaatactctataaaaacaacaaaagaatttgatgaggagagatagggatgttaacaccacagcaaaaagattcattaaaaaagcaacaaaagaaattaagtaaaaagaagaaataactgttgacaaatgagtgtacataggttatacttaagttaacaaataaagaggaggtatgacctatgttattcataatttttttcttagcagtattctttgtattaggatttattaacggatggaactcagaagactaaaaaaggagtggttatagtgaagttagaagataaagtattagaaagaattgattctcttggaggtaagttaggtgatattagccaacatgcttgggaagctttagtaaagtatcaaattatatatggtattatagaccttatagtaggtattatagttatagcattaactgtatttttatggaagttatttattaaccaacataaaaaggtaaatgatatagatagagatgatgattatagtttactatttgaagattgtgaagatttatcaggtataggtttgttttatgtaatagctacatcaatcatatcactatttgcatttatatacttagtatatggaatacctatggatattataaaaatactgaaccctgaggtatttgcagtaaaagatttaatcgaacaagctaaaggaggaaactaaaatgaaacaaagagattttgaatttgaggaagattttgtattaacttacgagtgtgaggattgcaaacattttgaagattggggtcatgatgaagagcctgaagaatgtagtgaatgtggtagtagtgacttaattaacaatacaagtcatgaagacactgagtgtgacatgtgtaaagggtacattgatatgtggcaagatggatatagatacatgggagataataaagcataccttgaaaaagaagattcaggtttaatttgtgaagattgttatgagaaattagatatttaataaggaggaaattaaaatgaacaaagcagtagagcaagcaagtagtgcattagggcaaggtttttcagctatggtatggcaccaggtgttagtaggtttaggatttattttactaggggtgttattatctttattagtttgggtactagtaaaaaaattacaggtaccatttaaccatccaacagcttttgtagtatactcaattatgttagttagtattgttgctagttttatttggggtggtttacacgtaattaaccctgagtattatgctattttagaacttaaagggtttataaagtaggaggaattctatgactaaagaagagttagagcaaagagtaaaagaacttgaagcagagaataaagaacttaaaaaacaaatagaacgttttgaagacgagggaggaaaaacaaaagatgaatagtagacaaaagaaaattctaacattaacaataaataattttttaatgttagccttagatactgtagcactaattagatataaaaaaggtaaaattaaacaagagaactataatacaggacagattacaagaacaatagttacaacagctaactcattaggtattctttacttagaagaacaagagcgtaaagaagttaaagatattaaaataggtacttttgaaactggagaccttaaaagatttatgaatacaaataaataaaaagtttaagaaacctattgacattaggtttcttttattatatactaatattataagaaataaggaggttaacttatgaaaggtattattatattttacaaagaagagaccaaagaggatttaggatattttcttgggtttataaactttaagctagaaggattatcttacacaactgaaggtactttagtagataatgatgtagtagttttaaaggataaccaaattaatgaggataatttagaacagtttagtatgtcaaacaataatttagttattggaatactaggtcattcatctctttcagtacgtatctatgaaaaaggtattagacaagaaattgaaagagtagaagaatatttagaggagttgagacaataatgttagcaatatttgcattaatttttggtttactatttattttatctttattaggtatttttatttattctatatttttacgaaagaaaaaacaattaatagaagaaagagaatcatttggtatttataatagaacgaaagaaaaactgggtgatgtaacacgtttagggtatgaggaagatgtatataagttaatccataaccaatctaataagacaatcatagaggataaaaagagtaaagttgtagatacaattaaaaagatgtatgaattagaattaacatctgtagatgtttctaaggtagaaggattatctccactcgatacagaacctatgacaaatatgaaattactttcatataagctagatagagaaggattatatagtttgagtaaatttatttaggagtgatacaatggaatttatagataaaaataatgtgattaaagcttacgatataccaaatgtttatttaaaaggttatgtattacaggcatgtgataaaaatggagatacaacagcttatgatggttatgaccaaatacactataaaaaaggtagagtattaacattcccttttgataaaccattaagaaagataaatgtactatcaggatattacaaactatttaaaaaggaggacataatatgatttattttgttagtgatttacattttggtcatgataatattagagaatttgaagcacctacaagaagtcactggaactcagtagaagagatggatgaaggtttaattgagttgtggaataatacaattacaaataacgatattgtttataatattggagacttctttttcaatatgaaaccgtctaaagtagaagatatacttaatagactaaattacaaagagatgatactgattgcaggtaaccatgaccataagaaacttataaaactatatgaacgtaatggtattacagtaaagtacgcagatatgattaaaaaggatggtaaaagattttatctaagccattaccctacgctaataggtagaaaaaacatgtttaatattcatggtcatatacattcacagttaatggatacagagtatcacattaatgtaggttatgatgtagagggtaagattgcctatagttttgatgatattataagtagagcaggtgaatataatggagaaattcaaaggtaaagatttatataaaactagaattagaaagcaaacaattaaaaatttagttataaaaacagagaagctacataataaacatggaaagtatagacctatcggtcatgtctattattacccgaaaacaaaagagtttactttatccaaacctgagcagaaaatatttatagagtacatgaaggcattaggttttagtgttaaacacaagagacgtaagaaaataattatagtatacaagaatgtgttagatgaatatcttagtatgtatcaggaagcaattgaaagtacgtgttgacaattaaggtatactatgttatagtatagaaaaggaggttaactgatgaagcattttattttgattttaggaattgtaattctagttattgcattaggtattgttttacctgcatggattttacaattagtgttatctgcatttggtgttaaagtaagtatttgggtatgtatcgggatatttatcttaatcagtgcagtaggaagtatgtttagtagaaattaaaggaggaactataaatggcaaaatatgaatcaaatatcaacggagaaaattatattgcaacaccatcacaagctttaagagaggcactagcagaattaattaaagaagaaaagaactttgcagattaccaaactaagggtgaggaacagtatgaatcacagttacaactaagacacttcgatgcaatgatttctcagtatgaagaagctattcgagtattagaggatagatatttatctcagatttttattccaaaagataataaggaggaaaagtaattatgaaagcagagtcaatagcacgattttttcaggataaggtattacaaatagaagggtataaggtaagattcactcaagctagttcatcatatattttagatatagatactatggatgaatcagtattgtttttagatactgtagttttcactctatcaggcaagtacttattagatacacatattacgattaataaacctgaaacactaagctctaaggaattatacacagagattggtaataaactacaagagattgtaggagaccaaacgaaaacagatatcgaattaactcgatactttaaggaggtaaaataaatgaaatcagatgctattataaattcattattaaatatgaaacatataaatatacaaaaacacaatataaaagtaacacttgagaaaggtagatataagggtgtagggtatctaatagagtttgaaacctcaatacaatacagaaataattataaattacaatctccaataattatatcttacgaaaatgaagagcctaaagaaatcattactcaaattaaagataaagtattgaaaattgtagaagagcagataaagacagatgatttatttattgaagcaattgatgatattaataaggagacagctcttagaaaactaaagccttatattaatgaagaatactactcagtgtttaaatcatctattgataagggagtacctgtagttttaccttctgaacttttagatagatgcacaggaaaaacatcaacaatggcttatttagctatagaatatgatttacctcttatagtatcaaacacagtcatggttagagtgctattaaatgattatccgactattaaagttacatcgaaaaaaagagaaggttacaaagactcaatagtgttaattgacgagcctgactctgaaatactaaatctttacttaggtatgcctgagtgtgatattattggtattacgaaaaaataaataaatttataaatacctgttgacaacaggtattttttatagtatactttagatataaagaaaaaggagaggttaacatgaataacaaacaaattgaaagattacacaagaaagcaaacgaggtagtaagtaaatcagaagatattgaagatttaggttatgtgcaggcacataaaattattaaaggatatcttgatttaccagtgtctgattttgaaaagagagatgtaatgactgaattactattagcgttagataaagctaataaatctatagattaataagggggtagtaatatggtagatattataattttaattgtcggtttaatattatttttagctagcggatataaattagttttaggtaaatattatgataacacagatttaaaaatgttatttacaatctttggtatcggtactatactattacttgtaggatttatattataaaggaggaaattataaatgaactataaagaagtactagaagttattaaaaagaataagccatgtaaggttagatttactggaagtattttagcaattgttaataaggaatttaacgcagatactgataaaggtgtgttacaaattgatgtatcaaatattaataaaaatgactacattaagttacaacagtattgtttagaaagagatgattatactgtagcaggagctattttattttaagggggagtaattatgaattatagagattttattacggattgtattagcgggggttataacgtccacattagtgttactgagaaaagagttcatattatttcagaaatgacatcagcatcttatccgaagaaagaaattaatttggatgaactgcaagcttatgtttattatatgaataattttggaagtcagattacaacggagggattataaatggaattagttattaatattatagcagtattaattggtatgtacggtatttacttttatgttacaaaatttagtactggtctatcaggtattttaattgtactaggtatggctgtaggtctttacttttacttagattacttaaatgttagagagaatgttattcgattagtatctgtaatgtttggtgctttcttatttagtatcgagatgatttataataagattatgttcgaaattaaaaaatctaagtatgataagactgttagaacgtacagaggagaccaataagaattttactataaagagtacttaaaataggttaagtgctctatatggtaccttaaaatggcttagaattgaaattaaggagatgaaaagttatgtatcctgatataggtgagttagcttataaattaaaaagtactagagaaagactagaaggtattatgatggaggaagtttgggaaatacatcatagagagacaggaaaattagtttttaaaggtggatacttagaagtaaaagaaatattaagaaaaatgtataaggagaatttaacattagtagatgtagacacaatgttaaatatcggtcaagggttttttgatgttgctaaaagtatatcagcagaaaatgtatttcaaataaactataaaaaggagttaccacgatgattaaagtattttcagaaatggatacaaaatataaaacgattgtaccggagaaattccctaatggagaaattaattttaaatatgatgacctaaaatatttagtagaaggagaactatggtttgatgttttctttaaatgggaaactgatgcagacttaatgcatttgtatatgtttactaagtatctagaacaattaggtattaaaaataatgtttccttcctggaaattgcgtatttaccttatagtagaatggatagagtagaagaaggtcatagcaatatgtttagtcttaagtatattgcagaatttattaacaatcttaattataaatctgtgttgatagcagaagctcatagccctgtcacagaaaatctaattaaaaattctgtttctataggtgtcacacttaaattattagaccaatatattgaaatgtctgaagaacctgtaacaattgtactaccggataaaggagcttatgacaggtacctatctgatgtggaagacatcttaagggattccaatattgaaaattattcaattgtatatggtgaaaagaaacgtgattttgaaacaggtaaaattaaaggtattaaaattgttaaggataaaaatactttatatgataactgtattatactagatgatttaacaagttatggtggtacatttgtaggttgtaaaaaagcccttgacaatcttaaggtaaatagtgtatcattaatattgacccatgcagagaaagtatttaaaaacggtaaattactcgattcaggatttaaagatattattgtaacagactcaatgttacctgatagccagtgggaaaaagctattgctaaacatagagcacaagaaaacggaacagaattacaaataaagagtatcgaaagatatctataaaaggagaagaataaactatgctaaatccaacattaatgtgtgacttttataaattaagtcacagagaacaataccctgaaggtacagaaattgtatacagtacacttgtacctagaagtaataagtactatgaacatagtgataatattgtagtatttggtattcaatcacttgttaaaaaatattttattgatatgtttaataaagaattctttaatagacctaaagaggaagttattaatgaatataaacgtacagttaaattcacattaggacaagaaaaccctgacgctaaacatttagagcagttacatgacctaggttacttacctattgatgtaagagctttaaaggaaggtacagtagttcaccctaatacacctgttatgacaattgaaaatactcactcagatttcttttggctaactaactatttagaaactattattagtactcaaacatggcaagcaatgactagtgctacactagcatatgatatgcgtaaaatgttagataagtatgcattagaaacagtaggtaatattgaagcagtagatttccaaggacatgactttagtatgcgtggaatgagttcattagaaacagctcaattaagctcagcaggacatgcaattagttttaaaggtagtgacactgtacctgtagtagactttttagaatcttactacaatgcagatgtagaaaaagaaatggttgttgcttccatccctgctactgaacattcggttatgtgtgcaaatggtgattacgagacaatggatgaatatgaaacatacaaacgtatgttaacagaaatttatccaacaggagtattctctattgtatctgatacttgggacttttggggtaatatgactaaaactttacctaggttaaaagatattattatggaacgtgacggtaaagtagtaatcagacctgatagtggagaccctgttaagattatttgtggtgaccctgatgcagatactgagtatgaacgtaaaggtgctgtagaagtactttgggatacttttggaggtactgaaactgaaaaaggatataaagtactagatgaacatgtaggattaatctatggtgattctattaactatgagcgtgctcagcaaatttgtgagggattaaaagcaaaaggttttgcaagtattaacgttgtattaggtgtaggtagtttctcttatcaatttaatactcgtgatactcatgggtttgcaatcaaagcaacatatgctaagattaaaaatgaagagaaacttatctataagaatcctaaaacagacagtggtaaacgttcacataaaggtagagtagctgtttacaaagatggttcatgggaagataacctaaatcttcaccagtggttaaataaacagaatttaaatcaattagaacgagtatttgaagacggtaaactatacagagaccagtcactaagtgaaattagagaaagaattaaaaataattaataaatatttaaactccctattgacaaagggagttttttattatatagtataactatagtaaataaaggagtgaaagagatgatttataaaatatcaaaacataattattatagtaggtttgagtattctacttaccctcctgatgaagggtttgcgtatgtagattatgtagatgtgattcttatcggtgtagataatcctaagaaaaaaaaggttattaccttaaaagtaaatgagttcaattcggatgactataaagtaggtcataagtacaatattataaaaatactatggtttgaaaaatgggaatggttaaagccataagtaaaaggagagaaataaaatgattatagataaattaaatggagttaaactagaaataagcggtcatgtcgtatcatttagtgtaagaaagtttgatacaattaatggtgagagacaattaatagactaccatcatattaaaagaaatagacaaaagtactttagaactactgaagaattttataatgaatataaagaaattaagcctgacaaaaatgaaatagatgaaatgtttgaatctctaggttacgtagatactgagttagatgatgtagtaagacacctggaaaaggttactgaaatattaggagttagtgaacaatatttaaatcagttatcttataaagctatagaagagtatgtagataaagtagttacacttgaaattaaagagttgaaaggagagaaatagcatgaataataactgggaaaaagaaagggttaactattgggaaaacgaagactgtccgagggaatacttagaaaaagcattcattgacctagtagaatatgtcgaaggagttacagtatcatctaaagatgttaagcagttaagagaagataaacttagagaagatattgggttttatgagtacgtagctgataaataaattagtatctacctattgacttaggtagatatctattatataatagtatacaaggagatgaaaatatgaaaaagttaatagtattacttacaattactatttctctattactaggaggttgctctcctgataaccatgagggtaaagtagtaggggtaggtgaatacagagaaccaactacttatataaaatcaggtagcgttactgtcccagtcattggtgaaatgaaatattatgtagatttagagacagataaaggagaagaccgtgtatatcttaataaagaggtctatcataagtttgataaaggtgatggtttctctaatgtaggtaagaaagtgtacaagaatgatgaattaatatataaaggagactaagtagtatgacacaatttatacatgataagaaagatagttataatagtacaaatcgtaattttgatactcaatattataaaaatatacctttacaacaaattgataggggctatggtcaagcaaaagctaggaggtttacaataaataatacgaaccaaaatatatggatacctatgacatatttaagacctaacggtactcttaaaaataatattgatatagattggatatttgttaaagaaaaaaatagtttaaagaaagcaggattagaaataaaaatagaaattacagaaggtggattataatgtatatactagagagaacaattaaaggttttgtaggtcaaaaagatgatatcttaccttattacttcagaagtaaaagggaaattgttcatttcttaaaattaatggaattccgtaagaaagaaacaaattattgggttaaaaagaatggtgattttactattataataagagctaaaaaattattatatattgaagaatatatacaaaaactaaaggagtgggaaaatgacttatgatgtgtttgtattatacaaaagaagagaacctattgcacaaggtagtatggaacattgtttagatgtctattattgggaaagattacatggatatagtaataaagggtatgagctattacctatagaatatgaccaggaggaacaattatgaatagtacaaaattagtagattattttacaagtaaacaaggtaaatatctaatattacctgatgaaactaaagttaggttatctaatgtagatttaatgtcttacattatgagactcacttttacttataacacagaagttgtgtctatagatattgataagttatattcagattctatagaaatgcatataccacaaggtctttatatgacaactgttgttaaaattcctaatacacagagtgttagttcagttcttcataaggtattagaggaatgggtaagacaagtacaaaatgatggtatattcggattcgtatgggaggaattttaatgtatagatttttattgtttagctattctgattattacccttgcggtgggatggatgatgtagagcttaagtttaatggtttttctgagttaataagtaacaaagatgatttgtatttaagtgaaaatattgaaatatacgatactaagtatgacaatatactatatattagtaacaaagaaacgatattaagtgaatttagagactatttaggataatggtaaaaataattaaaaaataaaggagaaatgtaaaatggcaagagaagctaaacaaacagtaaatacattattacaaagatacagaacagcattaagagcaaagaaaattatttttaacttaacagatgaccaaattgaagaaatggacttagaagtaacacttactacaaagtatagtaatagtgtgacaaatcaggtaattaaaggtactgacaaggatgtttcaatgcttatcctgattttaagtaacgaagaatttaatgataaggttattaatattgaaattgaagaaatcaatattattttataaaaactttaactctattaagagaagggcaactagtatttaattttaaatgggaggaattaatatgataaatagatataaaaaggtatgggatgaaataacccaacaaattgttaatgtagaaattattaactttaaaaatgaaacagtaacaatagaatctacagatgattcaggattatcagagataagaggttttgaagaagtagagtttatagattactatggataagatgtttaaagtatataatttataaggaggaaacatatggacttgtttgcaaaaataattattatgtctataggagttgtccctttaattacatttatggtatgtttagctataacagattattataatagacattaggactaataggagtatgttgactaataaggaagatatataggagaggagttaatcaagtaaaaccaataaaatcaattaaaaccaagtaaagtaactaaacaatacctagagcctgggaaaacttttacctctcccactcagcctattacttactaccgacttccctaactatgtattctatagttataatattcatttattatacaatacttaaactatagtattgtaaccttaatctatactgaagcggtattaatctattgttattatataataatcttatataatggtggtataatctaggttattacattagaatcattctaatctagaattttaatctttagaccctaggaaaagtggtactaaaatatagaaccctataggtatgggattcttatttttaaaattactaaaaagtattaggttttccctagggtaaagttttaatgtacctaaaatcgtaagtagctccttatcatttaggtctgtttaattgagaatattagaagatatccgcttcaattacaattaaaggttgacaactatgaagcggtatgttatacttagtattgtaggggtggttccctacagacctagactatggtcgtgattttggtttgcatattagtgtcattcataacctctattccagtaggggttatttttatttaagtgttgacatactctaagtagtatggtatgattagtatataaggagatgaataatatgattgatatatacttaggggaaaactataatactgattacttatctactattatgaatattataaagacttccgcttcaagagagttaagttatgggtttaataataaagaagcggatgtaaatatagaggatatgaagtatgaaggtgatgttctgcatgtatatggtacgatgtctatggattatccaggctgtattattatatgtattactagagatgagactaaggttatacatggtggtaactttctaacaggtacaggtcttagtcaagaagttagagaaaaattctagggggagttattatgattataccattaattatattcatgatgaccttcggtacatttgcattcagttatgttgcacatgatgcatacaaagtagatgaaaaaggtatcatgtatgctatggtagttggtattgtagttataaatgggattggtttagaaatgataattgtagaatgtttataaaggaggtagtaaaatgataggtcaaattatactattagctattgttagtatagttacaataggttgtgtaggattatttttctatgagacgttcaagagtattaagtatgatgctaatgataaaatctttatgattactgggagtatattaatgttagttattatcgttagtggtgcattagctatacttggcatttagagctcatttaagaagcggttaagtagttcatgataaattggtctagaaatatatgaccgcttctctatggaaggctgagagggtttagaattgaaaggagagatataatggctatattttttagtattttattattgattaacatatacttaatatactatgaatgtacttcagttacgtttgattggtgtgcactgcttatttacgtatgctcattatgctcttgtatttatttaatagtatattttgtataaaaggaggagatataatgcaagcaactatatactgtgctaataggtatagtaaaaatacattaaagagtcttttagaaaaattagaagcggagaatagaaacttaacatatagtactgacatatcagatatggaggaagtggatgttattgtacaacacactgacttatcttttgcagagttaatggattcttgtgatagagtaagtaaagggtctgaccgcttccaagtatttgtaggtaatcacgcagggtattatataaacggtgatttatatattaacgaagtaggtaagtttattgtacctaggaaaacgaatatgatgatgtaaggaggaaatataatgattcatatatttgtaaaagaggattataataaagaaacattaaggagtttacttgaatatattaatgatactgtaggtagggaattaacttatggtattaatacagactatgataaggatgtagtgattgaaaccgatgaccctatagatgaggagaatacgattgatttatcaaatcttcctcactttaaagatgacttatgtattcttatagaagaattatactgtaaggcatttgttaatggggaacctattattatacgtaagtatgtagaggggatgttataatgattataatatttttaactgaaaaatatgatgccaaggctttaaagaaagtattagaacatattgataattgtagtagcagaggtcttagctatttaatgggaaaaggagaagcggatgtatgtatagagaagaatgtattcagagaaagagatgatgtaaggattaactcaaacattattgatgaaggtaaactttgtatactcataaataaaaatggtttagaatgtagttactatagaggtgtatcatgtaatattggtgcctttgtaaaggagagattataatgatagaaatatatctcagtgaaaactatgatagggatttactcaaagcagaattaaaatggattaaagagaccgcttcaagagaactaacttatgatgttaatagaagcccaggattagatgtacatattagtccttttagatataccaaggatgaggttgaagaaatgagcttacatcctcagtttgaagatgatgtatgtgtatttatagctgaaacatggatacatgagtatcgtaatggtaaatcaataggtgtagatagtatggaagaatatgtaaaggagatgtaattaatggtagaaacttattatacagtatatcataagaatagtatgaaaactattaaggattacttaagtagaagtgatttaattgacttcttatataagacttggcataaggatataagtaatatatacccaacagattatactacgtatgaaacaaaattctgtatagacatagatgtagttagattaattgaagcggttaatgaagaaggtgtattacttattaatgaaggtaatattgtagagtggtaggaggagataaatatgagtcagtttttcacgtatacagtgtatcatacagaaacagctaaagtagtagctaagaatttgtttgatgcatcattggtgcaccttattaacaagatgatagagaaagatatagttaaggatattaatttagaatattacatgaatacacgtggtgtagttatagatatgagtagactactggatgatatagaatcacagggtgtatttctattcaaacgtaagctacatggataataggttagaccctaaaaagtttgagatacaataggatgcttaggataataggataaaaataacttaggatgcttaggatgaaatacgatacgatagggaggttaagttaagatgcaagagttaataacttatataatttaccataaagaatcaggtaaagtagtagcacaggatttaagtgaattagaaactattgaacttatatacaaaatgatagaagagggattagtaaaagatatagaattatctaactaccgtctttttagtgggaatatagatatacttaacttattagtagacatacgtaaacaaggtatatttgattttgaacatacttggcacacaagataaggagggataggatgatagttatatattcagatgagtctagggattacttaagagatgagttcttaccttggcttaatgatagagatagatatttaaaatattataagaatgagttacctgaagatatagactcttcttatgttgtatcagttgtatactgtagggatatggaaggtcttatgaataggaaagatattcttattggtaatagttatagagagcctgtagctgtattaggtgttcctgaattttttggtaactattctaattattactattataaaggagagcatataagtaagcatgacctaggagagataattaggttaaaggcttggcaacgtaattttgactaagtagctctctctaatttcactaagtagctccctaggaattgcctaagtagctcggtgtgattttaccctaagtagctctctctgttttctattagtttattttaaccgcttcacgtgtctatatatagacaatcggaataatagcagaccgcaaaaagaaatacactaggatattattcccagtgtattgtataatttttttatagaatatttataacattgtattcaaattcatttacttcatgttgtgatttaattaaatttttaattaatccgttttgtgttttatagtcttttactagtttttcattttctataattaaattattaaattcttcttttgttgtatcctcatctacataaaatttactttcatatatttcataatattttttatctgttccgttatcaatatcatctgatatttgataatttttgaatacaatttcttttgtttctaattcatttactaataattgtgattttgcatattgtaatacgtcttcattgtcccacattggaatatagtttattttcatttaaatcaaacccttttctataatttttttatataatatttgtagaagcggttggggtttgtcccttgccttactacacttaatatattacagtatagttattcagaagtcaatacttttgagtaacttttttttaaattcttttttcttctatataatagtagtttttagccctaaaaaaagttttttaaaagaatttacattttcttattgacttattaatcatatgatagtaatataaaggtacagcaagagaacagcaacaaggtattagaattatataaaaaaattatttaattggagatgatttaaatggatgtaaaagaaattgcagacactataatggagttgtggcaaatggacggatacagatgtacagaaccgccattatatgaaagcacattaaaccacacacgcacatatacggctttaatcgtaagcattaagggaaactatgacactgttcaaatgttccgtaaaacgcctataatgagcatgagagggcaagcgcaaccggctagcatgttagtaaatgtaattgatgacgtgattataatcgtatatgaaaatacggtttacggggtacagaataaagaaataaaatttattgaagaaatttaaaaataggggttgcaataccccttaagatgtagtaatataatagatgtaagggatagcaacacaccttaaaaaactttttaaaaaagttaaagaaaagtgttgacaccttacaagatacatgttaatattagtatagaagttaagacaagccacatagcaaataacgaaattaaataaaaaaattatagaataggatttgattattatgacaaacaaaaattacttatacgaagaaactcacacagtacaagggcaagacattacggctttcagaattccaaatgacacaaacggcaacccacgttatgtagtgcatttcatggatttagacattaaactagcagactatgacaacatcaataagctatacggatttaaaaaatatactgctaaatggtttggcggtggtgtagtattccaaagctataatattgaagatacattaaattacgcactatctaatgttaaagaaatagaagcggttaagaattaaaaccgcttctgaattaaataaaaaaattatataaaaaggatatgatattatgaaatttaaaatagaaaaaaataacagtgatataaaaactttatggaatttagctaaaaatggatatatgagttatcaaactgtacacaatatatttaaaaatgaatcagatgaatttattatatttaacagtaaacaaacttataataaatttatggaattaagatataatagaagtgcaattcaatagtataaaaaagttatataattccctaggattaaattcctagggatttttatttgttttaatttatataaaaaaattatttaataaataaattagtgtaaaattgactgttgacaaggttgtattttttatggtataatgaagtgaagaccttttttagtataaaaaaattattatataaaaaatttatattaaatggttttaaaaccgctctttctccccaccttgtcatatttatagtggaagggttaggcgggttaccactgttttactttctatatatagtatactatgaataatggtaattgtcaacacctttcagaaactttttttactttcttttattattatataaaaaaattatacatattttagggctccacttccattatataataattcggtcttaatgtcaatacttttattcaaaaaagttttttaaattaatttcaacaaatctattgacttatgtttttttctatagtaatatataggtataccaacaagggaggcaatacaaatgctaaaatttaaatggaaaaacaaaacaatcaaatcaactcaaaaaacggataacattattttacttcttataggtggtttagttgcaacagtcacgcctaaacttgtaaactggtttttactactacaagataatataaatatttttttaagataactattgacaacttagaaacaacgtgttaatattaagatacaaggtaagggaagcggtcaaccgcttccgacctaaataaaaaaagtttaaaaaaactattgacagtcacttgaaaccatgatattattaagataacaaaaaacaaacagaaaaggaattgattataatgaaatttatcaaaactatcgaaaacttattagaaaaagcagaaaacaaagggcaagcaattttaaacggtcgttactttgacgggtatagcaaaggtgagcttgaggaaaaatatgcaattgaaattgatggcaacaaattgattatgcgtcactggggtacacaaacaattgagattgacttaagcataaatgagattgtttcatattacggtgaaagcaattcagaccgtgacagtttaaacacacttgtatattgcttaggaattgcgccaaactttagatacttaccaagcaaaaacttattcatttacgaaaattaattaaataaagggcttgacttccaagccctaccatgttattattaaattgtaaggtaatcaagcacaacgacaaaataaactgaaaaggaattgatgaaaatgttcaaattacaaaataaagtggaaattatcgtacctaaggaagataacaacggcgttgagattgcagacaaacgtattaaagaatatgtaaatagcatcacaatggaagcgggcggttgtactattacagaaattaaagggcaatggtactcagaagatgaaaaacgtataatggaagataacaacttaaatcttgagtggtattatctgcaagacagtgcaaaattcatgacagttgaattaaaaggtattgtaagacgtttaattgaagtttacggacaagaggctatcagtatcaaagttaatggcacattgtacattgtagaccaatcagacattgaagaattacacacaacattattaaatatcatgaaataaaaaatttatataaaccgcttcggattaaattcttgaagcggtatttttgtgtaaaatttatgcttgacaaatgtattaaaaaatgagataatagagtgacgactttttttagtataaaaataatattatataaaaaagttatagagtttttagggctccaagtccattatatcaattttgctactagttgtcaatactttctttttttatataataatttaattatcttaaagataccgtccacctccattatctcaaattttgcccctaaagtcaagaactttctttcaaataatttatttaaaaaagtttacaaaaagggttgacttattttgtactatggtgtaatatataaagtgtagtaaggaagcggaggaaataacctaaaaaaagaatttaaaaaacttttaaaaaggtgttgacaaacttccaaatacatgataatattaagatagttagaaaaacaaaaaaacgaaaaggaattgataattatgaacagattagaaatagtaaaagatacggcaatggaatatatccttatgatggataatagcgttatggacggcgttatgacacaagaggaatacaacgaagcggttagctttgaaaaggtgtatgactacactctatcagaagcaaatcaagaatgtaaattcttaggtggtaaggttttaactttcctagtacatgaagcaatcgaagaatacgcataaaaaaacttaataaaaggggttgacattcaacccctaccatgttaatattaatatataccaaatgagaggaattgataattatgagatacgaaattgtaacgttagttaatcaagaattgtttatgtatgcaacattcaacaagcaggaagcagaagccaaatatcaagaatggtgtgacttatatggtcaagaaaatgtaagcatggaaaaaaattaaaataagctgttgacaaactaaccgcttcatgataatattaaactatactaaagaaaaggaaatgattacaatgacaaaaacaatcaaacaattagaaagccaattagagagtctagaaagaaaatcagatgaggaattagcaaacggatactatgacagatttgaaagaacttgtgcacagattagagaattagacctacaaatcgaattaaaaaagaactcagaaactgtttaaaaaaatctaaataaggggttgacacttaaccccttacatgttaatattaagatagttaaaaaatacaaataagaaaaggaattgatacaaatgaaattaatcaatagagataatgaaatcgtaattagcatagcaacacttgagagcgtaaaacaagccctaatttgggagtacatcgaccacttagataataacatcctagacagtaaaatttacgaccaagaagcggttgtggtgacttctgacactctacagtcaatcaaatttgcggacactatggaagaactagaagaatatgtaaacgacatcggttggaaattaatttaaaaaaggtattgacaccctagcatatagatggtaatataagagtatagaaaaaataaaaaaagaaaaggatttgattattatgacaaatacaatacaagcatttttacaaggacaagaagcaagcacagttaaggacgtagcaactcatggagtacaaagcggagcaattggcaaattaatctacacatcagacatagtaaacttctttgatagatacgagcaggacattgaagccgtcatcactgaatacattgaagaggttacgggacaacaatattatgacttattgaactatgagcttatgagagacctcgagaattatgcaaatgtagaatttgaagacgaagacgaatataataacattcaatttgacctagcagaaaacattgcttctgatgaggttgaaggatttgaagacatggacgaagcagaccaggcggaagcaatctatgaggctatggatgatgttgaattagaactacaagaaacggacaaggttcaatatgttaatctagcggttgagattgtagcgcaaagaatggcactataggaagcacacagacacacacagagaagcttaaccgcttctctaatacaattaatcaggagatgttgaagatgaaaattaaccaattaaacaatgctaaaaaactatacgaaacattatttgctatcgagtttgatagtactatcgaggaagattggacaatagataatgactctctagaagctaaactattcttggagttatcagatgagggcaaaaaggtttatgcaatgagtcacgctaagcaatgggcaaatattgttatggatatggaagaagaggaatatagaagctattacggaaatgatttagattatattttagaggagatgtaaaacatgaatacaagaagggtaaacaaagcgttaaacgaagcggttcgattattagatgagcaaatagcagacactcaaaagactatgcaggagttgaataaacaactcgagaagcaaataaaggctaagcaagagctaatgatattagttgatgttatgactggtgatgatgagtaatgagcattagagaggttcataatgtcgttaagagtgcaaagagcaaactcctgcaggaacataataatattaataatgtaatcatagatgactacatcacagaagagctacacagacgcacacagggaagcggaacaatacagatgaacaataacaccgcttcatatagtaatggctcatatggtagcttagaagagcttagagaagcttatgacctatcttcattatctactgaggagattaaagaactaatacaaacatttgtttaaattattttatcaaaacgctttacaatcttttaatttgtatgatataatgaacttaacaaattaaaagaaaaggaaatgatgaacatgacaaacttattagaacaggaacaattagaaaaagacgtaaaagatattatttgggtattagacagaatgattgctagaggagaacaatacactgaagcttacgatattttagttaacaaattagaaagacaagaaaaaagaatcgtagaaataaaaaaacaaaacggaatattttaaataacaaatagagggaataaaatccctcttttatttttatcttattatataatttttttatattatacgggggcaggggtaaaatgccactcaatgggggtgggtctatatacccctatggtctacccaggtacttattttttggggaaaattatgaaaataaatatttaaaagtcaacacataaaatatagaaagtaagtcaacatcccttataataagtcaacaatttatagtacaaatataaagcctctaaatataaagtcaacatatctaaaataagaagagaggtatatcctctcttctcttaactatataagtacgcttctattatttagttcctgttctattctatttaaatgctcccaaacctcaatccaatttactcttgaatcatcataataaaggtggaaatcttgagggttttctaaatacgcttctagttcatcatagtatagaacatcaaaagctgtgtcatcaagacctatttgaatctttagtatatctttatatcctaattgtttattaccgcaataagctgtatcttctaaccgcttctctagtttattaaatagtgtttgtataccttgtagttcattcataatgtatgacctcctaattgattatatacttagtataaaggataggtgttagaatgtcaacctttatatataaaaaaagagaaagaatattattcttcctctaaggtattactaataacttctaattcatgaatagtaatcatatccattctatcgaatcctcttgggtctcctttaatgaactcttgttctcctctatgagttatactttctttatatccttcttctttaatacgtttaattaagttctccttatttgtgtatatctcatcttccctgaaatggaaattatcttcatagggttcacaattatcatgttctacttgatatagtttcatattagttatcctcctattctctatagtaatcatatatttctaaataaggtgaaaatggtgagtcacctctagtattaataataacttctccctcttgattacttaactctataagctcttctaagctattaatttctacacaccaacgttctatagtaaatgtattacctttttggtctacacgttctaattcctttatgtaggcaccttctataggctttttattaattgtgcttgttctatctataaaaaattccattacttttcctccttatacacttatacgcatgattaaataaaaattataaaacttactgttatgatgttctatattacaaaggtataacattagtctataataatactctaaatcatgagtatgtttaaaagttttttcattaaactcagattccagtaggttaataagtttttcatgagttaaaacatatgcaccctcgtcaccaaattcatcaaatttatcataatatatttctgcatctcttagtaagagttccataaaatatcctggttttctaaaatcatcttcaaaattaaatctttgataatagtttttttctaactgatttctatatttcgaattgttaataaaatgttgtaatcctttttgttctacttttcttttacttaaagttacaccaattacttcaaaatctaatcccattacttttcctccttttcgtaagtccattcaccatattctgcgttaaaatgagctgtgtctgttccctcatcttcatattctacactataccatgcatcttcttctgtttctgcatctatatatcttatttcttctgtagtaatagtacgttttaccttaaatctctccatttgtttttcctctcctctaccttcatagatattacctataacacataattctaaggtatttgtatatgattttaaatagaaatcactaagcaaagtaatcttattaccttcataattttctataaagaaaaaccctttaccttttttaacgatacctgagtttatatagttaccattaatatcttgtatttctacaatatctccctcaaatatttctacactatttctatcactaagacctgtgaattgcataatttcaaggttttctcctaaataaccatcacaagcaaatagatgtgaatatactgtgtaatctgcaaagtcaaacctagcaatatcaaacattctttgtgattcttcatcccatgctctaaatttaatcatctactttactctcctttatattctcttctaatatttgttttaatgtttgacaatctttttggtctagggtgtcccaactctctagattctgtaattgatagtataattcatttacaatttcatcgaaggcttctgctttttgatagacttcttgtaactcttctaattctttttcattatcaatacaccagtagttctcgttgtcagttataatatcttgaattttatttttatattcataagccattatttatccctcctatcttctatcctttgttttatctcttctcgattacggtggtgttctttactcatttctttacgttccttatttgttaatcttattctataaataaggtagttaatatataatgttccggctgaccatagtagtaggaatgatattaggtaagtccatgagacacttatttctatcattgtgagtcctccttatattctttataacttttaataacaatcttacagatacctctattaacagctaagaataacataaatgataatagagttataactgctctaatgtctccagtaaagggtaataatttaaatagtaaaacactttctaaaacacttgtagctgtaataattgtaagatataagatagatagtaaataatcctttaattttagtttaacgaaaggttttttattatcttgagttcttgttataccatatagaattataaaccatgcaacaggtatgagctgagttataaagtcattgtttatgcctaatacatataaaccataaatgataccggcaggaataccaataatgaatgctaagaatacagagaatataattagcattaaaggaagagctacaagtaatcctaatccttgtattgaatactctagtgtattctttcctaaggctttaaagaatgttttattcatcttctacctccttgtaatatacagtatctatatgagtaatattatctttaaaccatagagacttatcccctttgttagattctaaagttttagcaccattaaaaacaagaccctcaataaaagaatttaaattagttttagtatctttttgttgtacgataatagaagaccctgatacatcacaaattcttataaaattaatatctttttttatttctttttctactttattatttttaaatatcattataattcctcctctatataatcaactgttttaatatactcactattttttagtacctttttcacattttcactaataaatttttcttgctctatttcatctattttttcctgtattctaagatacagaatttcaggaacacaggcagggttttgtaattcttcttctgtaactgagtttgtaaaacttgccattccatcattagctagaataagaccatctgtaattctagttaatttatctccattaggtttataatatataacttcgtaaacctcttcatctttaaataactcttcacgaggtattcttctagaggagaagctataaccatttttaaaggtgataattgtattttgagtttctcttaaaaatactcttaaagtattttcaaaaatatcccattttttcttttcttcatctgttatagttatattttccttgctttttttaaacatcgttattattcctcctcttcttctatagaattactttccgtaatagttacttctagcatactattgtaatactcattcttttgattaatattatagttatcattatattcattgaagtctacataaatgtattcatttgttccattttcataaataatatctatagctgtaatatctgagtacgctgtaatcatttcataagcatttgtattatcagggtaagcaaaaccaacttgaggtatttctttaggcttattaatgagaataccaaagtaagtacaatgacgtgttctatttatgtgtgaagttcctttatacgtaccatagtaatttattccttctgtaacaccttctatatggaacctgtttacatctttaggttctaaccttactacatcacaattttctaatactaagtctatatattttatattcattttaattctccttttttatttgtatttgttgtatacacaatgtactcgaacgtcttccattactttacctaatagattctgacctttccagttactctcatctaatattttagggtcactttctttaagacctactccccatattttatcataagatgatgcctctacgaaatctttacgtaaatctgtatcgagtatttcccgttttaagtgtgtagtcataaatttatctttaaccacttctaccataatgtcatatcttaccttattccattgctcttcattaaaattacgaactttacgacctagacttttagcatggttcggattcttagcatttaatatttctcctgctatttgccagtctttaaaatattgcgctttgtgccacataaaagcttgttctgagttattaaatgttctccctttatgtttaaatgttattgggtaaaagttagaataaatatcttccttaccccaaaacataatgtatctgcttgtttctttcataatatctcccctttaattccataatgatggtaacacaattttgaaattatctaatattttattttgtacttgttcaagctcatcatatttatccatatcaaaatcatccatttcttcatgataatattgtattaagcttaaaatatattttatcatatctatttgtgttctttcttcaccatctacatctacaaaagtattccattccatatctatacaagaacgttcactactttctataaaggcatttaaatcggcatataattgaacaaaaaaagacatatcatagttccaatacttaggttcatttcttcctaattttttattcatttttttatactttctatttctctttaacccaaaaacttctttttcaaaatcatttaattttaaacctttaaaatattttttcttcatatctaatcctccaatttaataagtggtaaatctatatctctaaatacagaacctacgtcacatagcagtatatcattatgttcttctacttcaccactactagtaggtgtatgaccacatacatatataaatccatcttttctaggttggaagtctctagaccatattaactggtctactgtttgctcttctataggtttccaactaactccccctgaatgggaaaatatatacttaccttctttataataccttctacaattaaccataaatattttaaattttctataatcttcagattctttaagtttctttagttcacttttaataaaatcataatgatttcttaaattatcttctacacttttatattttaaagttacagtactaacaccgtaagagttaagtgtttctatacaataccttgataaccattcaatatcatagatacttaaccggtctacgttttccataatattataaaactcatcatcgtgatttcctaataaagctactacattatcatcatttgatagtaaatcaaacatgtagttaacaacgtcttttgaccttttacctctatctacataatctcctaaaaatacaatagtatcctcgggtcttcttttttcatttatcttatccataattttcagtaatttattgtattcaccgtgaatatcaggaaccacgtatatagccatttaatctcctccttattgtatataactatcttaccatacttatttataaaagtcaataaaaaaacacctattaatgaaaataggtgtccgacagagctcccgtacttagattacggttaataatattttacgacaattatatgagaccctctatcgttgaaacgctcgtcactgcgttagatacggaatggagggatactaccatccggagtctacggtcagatacaaagcctctgccgggcaacatacggtatctctcgtacatcaggttgactagacctttagagattttcactccttctcttataaccagtaacttaagagaaataggttttacttagtagatatgaaacaataaatctacatataatattaaatcatagtcaagtgattgcacatatgtctaatacctataagttttttgctagcctggtatatggactctgtaggattcgaacctacagtcaaaccgttatgagcggttggctttacctttaagctaagagtcctagaaatatcctgagagaggactcgaacctcaacgactaggtagctacaactagccagtgccattactcaggattgctagtaacgctaaatagagtcataacgttaccgtagaccttttctactcttggtagataggtaaaatataatgatttcaaagtacctatatagtcaggctcttactctcattataaggttaaaaaggttaactgtgtttagcattatataagaggctttagttaactactatactaatagtatatcataaatagtacttagtgtcaagataatttatcaattgaatccataatttttgatgtacttcttatatccgcttctttactgtgtttaagaagatattttaatttcattagaaaagaattcttttccttttctatagtatctttcttgttactatattctgaatacatcataaattctataaatatactatttctgtcagatgaaaacatatctatagaaaatggacaatcaaaacttatgtcatctttattaatactaaaacattcagtaacatttaaatcatttatttcatatacctcgaaatatccatcaactcttttaagttctatagcactattatatctataataacgttgttcctctattaacttatcttttgttagataaggatattcatttataaatataggattacttgttccatagttatttttaatatattcagcatcttctaaggaatcagtataacctaaaacttcgtaacttgttgtatacacagtatcttcttcccacaagtcatagtccatttcctctatttcttcctctagtatataaatttttttcatatattactcccaaacaccaataagatttttaagtttagctataacctcttcttctgtttgataagaaaatactcctgtaatgtgttcatagttacctacaatttcataatcctgcgtaccgtgtctgtctactagatacgagttacccataacatttaaactatcttctgaataactgaaatttatattatagtctactaaaaaattaataatttttttcatttacataacctctcctatcggatattgtcctaacattcttgttccattttcattataaaaagtatattctactacaataatattcatcatatctacatatatagcttctatataaggtttaatattttcttcttcttgtatatgcttacctataatatcatataataattcagagtgtattcttttatctctcattgtagacctccgtaagaaatgatacagtcttatcttttaaagatttttctactagttccatagcatctttataatgttttatgctagattcattagatttaagtttatcttttacttcttggattaaaggttctactttattaaccaaatcttttttcttttcaatacttacattacttctcttattatctaatacttcttttggcatatacttaacttttgcaaagtctttatagctaacatttaagttatctaaatcatctaataaatcattataatattctaaatgattatagaatgtgtaaaacttaacaaggtctttaccagttaattcttctttttttaatatattattgatattaccaataacagagtatgctataggcttaaaattagctctaacataagttaaaaatataaaatcatcataaaacaagtctaaaacagttttattgaatctagtgtttttagcttgctctaattgagcacataaattaagaacattatcaaatccactctttaataccaaagaaataaatctctccactgcatagtatcgagatacttctgtatgtttacttgccttttcactatttctaaatatagtatctgataaaggttgaactactaaactcatgtaatctttatctgaatactcatctgatgttccttgataggtacttccaaattctattgttgataataagaaacttttttctaagttcattataacatcctccttttacttgttatttaaataataacatatgttgatagtaatgtcaaacattaaatatcttctgttgtgtcaacttcatcttgtttatatttaaagtgttcataaactttaaatagaatagcccctagtgttattaattctaaaatatatttcataacaatcctccttaataaccgtgtttagttacccatcctgctaaagcatccatagccatatcatattcttcttcatttttaattcttataattttctctatttcttcttttgcgtgtttagaattcataaaatcaatatctactgtatctaaatttgtataatcaatattttcttttataaactctttttgcggtagggttatagaattaactcgtacattttcgtgatttaaaaattgatagaagtccatactattcaccttcttttaaacagtctgccatatcttttaaagtattaagtacatatttcaaatctctataataactatcagaaaaactaataacagcatatggtgtcatatcactatagtgtgcacaatcaaaacctaataccctatagtcaccttcatgttcatcgtatgttatacctccatgagaacaatcttctatactattaaactgttctttggttatattcgtaggcacattaatataaccatttagatgtcctgaatgagggtgacgtttaacagttaggtttattcccttataatctatatttaaagttaagtcctctcctaatatattatcctctttatctataatttccataatatgttccggtgcttttccaaacatcataattcctcccttttatttatacttttactatacactactttttctattttgtcaacaaaaaaaggctactaattaaagtagcctaagaattaattatttagcgttatatttccattgccaataaccatttttctgtgagaactcaaagtgaaaaccgtcatagtcaaattcaatattatagtctccatcttgaagtggttttgaatttagtacaggactattactctttgccaattctgctagaaactcatgatttactttttccatagggtttattcctcctaattattcttacagtactaatatatcacaggtctttttctaggtcgtttttaaacttctcctcgtaagagctagcataagtaacctcatatcctattatcttagtataatctatgcaaagtaatttataattggattttactttaatatcttctgattgttctattttgttgataatttcatttaattcatctgaagagtagtgttcattatcaacttttattgtttttccctgggtatagatatcaatttcttgtatcatcatttcatccttttgattattcattatttgattataagtttctaaatcatcaatattatctgtatctgaaccttttactaaccattctcctctctttttaaggaggtcatcaaacttctcatgctctttaattatcttctctacttcactcggtattagaacagctctagcgtaatttatatgccacatagacatattatcaataagataattaaccattcttataagttccttctcatttgccatataccaacctccttatatctaatactaatataagagaaaagcagacttattaaaagtctgcttctgtacctaattctaatcttctatttttcatatgaggaatcgtttttctatctcctgttaatagagataattctctagctttttctttagataatgttaatagtccattataattatctactttactattatattgtcttactaagtactctagttcatcttctatatctgatagctctcctgatttaactccaagtaactttctatacatatcataatcttcagaaagactttctactttatttttagatacagaatcataaactgcttgtaaattaccttcttcaataagtttaaagttatgttcacctatgattaattctttctcagaagaatcaagtgtcactaacccacttgtattacctgtaaagtcacctttataatctacaacaataccttcagttactttgtcacctaattcaatagtcccatcttcattttctttaaatttatgagcatcatatacttctactttgtcacctaatctcaaatcttgagttaagttatgtttaccaataattctatccattactcaatctctcctttattaatagggtcttgtgttaagaacatttctagattctcttttgtaataggtaaccaaaaatatttactttccggaattgtaactgtatagaaatcctcatctttgttaactttaatattaacatctgtaaactcatcttgcattaaccaatgagttacagttaagttatatgacccatcactaacataccctaaatcaatatcatgtttaaaagctaaatcttctaagtgctctaataaatcattcttttcattttgttcttcttctgtattatttttgattggattaattaactctgtacaaacgatatcatacaactcaccatctgtaacttcataattcttttctattaatacatcttgtattttattgattgaatttgtaactactgctccatattcttcttctgtaaaattacatttatctaaatcaacatctgtaattaattctgcaatccatttatttaaaattgataccgccattgttctagaaataatactatcatataccatatttatttaatctccttgtttaggtgaatgtggtcttctaatgaaaaatcaaaaggcgctacaccatttcttttattgtttgtttcttttttaagtataacataagttagtgaaaaagtcaagatagttactacaaccattaataaaagtttaatcagattcttcataattactctaactccttaagtttattttttactttttctttatcatacttataatctttttctgagttttcatttttttctttctcttcttcattaagttctctgtattgagcttcttctacctcttgttccttattatctttgcctgcgtctgctttttgaatttctacatttttactactaccgccactcaccttttttctaaaaagaaaccaaagtattaataaaatgatgagtaaaataataatgcttaatacaaccgcccaaatattattagccattacaacctacctccaaatagtttttttacagctcttaagttttctgctgactcattgtttatatgaatacctgtacttgaatcgaaaataacagcattatcaagtaagtgttttttaagttcattttcataacttcctttacctactacactaccataaccatcgtgtgttaaatcagtcatatcagattcaacttctaatactctaaacgatactctacgtaaaaatgatgggtttactaagtaaaaggatgatttaaaaacatttaatctttgataagaatgttttatattaacaacaaacccagtaagtttatcattataacttgagtttgatagcttacctaaatataaatttatactatacccttttgtttctaaagtttgtatagcactaagcagtatagctcctctataagcaagattctcagggtcttccatccaacttatactagaattatagaatacatcaataactttcttctctgctttaactctctgttgagacatcatagaattaggtaaaccttttatagcgttaggtacatgaggttgataaccttcaggagccacaactggttttctttttacagatttatccattctgaataatgcatctgtcatctttttaagcttaacaaccatatcataagactctctatctcctttaaccattaagttataggcttcattaaagctatgagtgcctgtgaagtcgtagctacctgtatctgatgaattgtctctacctgaaactctattcttttttaaagcagaaaagaaatcaggtagaccatcatatttaattacatttaattctgagttatctattaatcgtctacccattgttttttcctcctattctaatcctagtttatccatgatagtgtcaaagtccattgaatcactagatgaactttgagtttttcttggttcttgtttaggttcttgtttgatacctaaaagattttttgttgcttctgtatatctgttatctttaggtaaagaactaataaattgattaatttcatctttaggtacagacttgaagatgatgctttctacaacaaactcatcgtccattactccatctaatttacttccattaataattgcacgcattgaaaagacataaggtaatcctttttcatcgttctcatatcttaatttttgtacaaagtttactaaatcctcattacttgatagttggtgttctaccttattatcataatcaaattcaacttgagcaaaacggtctaatgtagctccatccagttgctgtcttcccacataaatatggtctgctccagttcccatagtattacctgctgatactactctaaagtcttcatgtgctgtcacacgaccaatagggaagtcaaagtatttatttgcaatagctgagttaagaattaataatacttcagggatagatgcatccatctcatctaggaagaataatcctccatttgtaaatgctttataaaactgagtttcatgaaacttaccatttgcatcaataaatcctgttaatttaaattcttgagtaattgcattactgaaatagaaatctaagtctagagcttctgctacttgctctaatacatggttctttcctgaacctgctccaccttttaaaaacactggaatattttgattaactagttttaatatatcttgatatctataatggaagattcctgagatatctttaattgttttaccttcttgttgtaattcaatcttaactggtaaattattaagttgctcctctacgtattcttcaatttgttttttaacatcagtaataattatttctctactctcagttcctgctttctctacaattgcatccacaattgcttgttcataagggttagagtttttctctcctagtttatctgctaagtctttcgttgtctgcatttgttgttctaccaatctctctaatctttcaatagtatcttgctttgccatatttatcattctcctttgatttgttatacatttattataatacaagtattttcatttgtcaacaactttctaaaacttttttagttgttaataaaaaaattaccttatacctataacttaacatagggtaaggtaattgtcaacacttttatctaaaaatacattaatttaaaaaagtcatcaatgtcttcagttccatgtgtatccatattatacataaacatacaattatatgtatgtttattcactatttctagcatattgtgcatagaagttgcattattgaattcttctaaatcaatagttatcgtaagttcttgaccttcgtaaagtatgtttgctatatagtatgttttaacaccttccattgttccgtgggaagtttcattatgattaagaacttctatacctaatgaaggtaaataatctgaaaaatagtatttgcagaaatatataaaattgtctgttcttttagacacgagtactatccccatattttatatttctttctaatcttacataatatgttttaatcttttgaacttctttgtctactgcatcttttcttcctaatcttgtagtatattttacaatattaaatatcatagaatcaacaaaaccatcataagaaaagtgttcttctagaaaagaaataacatccttactacctttatagtgctcaggtaaatgtgcatctacttgtatattatagtaatcttctaaaagccctatactttcaccaagactagataaagcataacctaaatcattagagtcattagaccattctttagatactgataaagcatcttctataattgttatttttaatttatctaagtaatcttctacttgagcttgtgttttcataaattcttttgcattcatttaataccctcctaaattgtataaaaaaaacaccctgcttggatacaagcaaggtgaaaaaggaaagatattatggaagtgtactatctaagtacacctcataatataacagttttccttgctagttattacttattttttaaggtcttcttctttaacaaacactccgttaataagcttacctttcctgtcttttatctcatcataagccatatcaatacactcttcaatatctatatctaattgtaaacatagtactgttaatactacaaaaatatctcctacactatctcgtgttacatggtcattacttttagcaatacctgaagctaattctcctgcttcttctaataactttaacatttgaccttcaggtttacctgtttgtaaatttctatcttttgcccattgcttaataagttctactttttccattattctatatctcctttaatttctgtatcttttataattagattatcagaatcacttattacagttaagttatcttcgactaattcatgtaggttattagtaatatcttcttcatacctataacctacacgaacataagctttcaccctgacatctatattaacataatcttcttggaatttttccatttctaacttcctttattataccatattgttatacctttgtcaattaatctgagtagtttcctttggcaagttgatactttttgtgtaattcttcatataattctctcataccttcgtagtttctcatatcatcttccaagaaactaagataatctaataataatttagtttcttctacttctaaagttataactggtttgaccatttatgcaacctccttaaattcttctttatttattttcttaatatctttttctaatgcttcttttagttcattaggtaatttatatgcatcaattgattgttgctgtcctaacacataaccattatctgtaatacgtatttcaactgtaaaccatgaattatctaaatcttcttctaatcttgctaataatattaaacagctattctttagaattctattagcatatcctccaacacaatgagataacattctaccttcatcttttaatttacttacagtatctgcaggaaggaattttacttttctaccatcttttaatttataagttttatcaattattttttctaacttattatcatacttagctttaagttctgcatcatctaattgttgttgaatggattgtttttcatctgtaactatatcatgctctaacttcagtgaaaaaggcgttaagttaacactctctaatgttctatacccttctcggattagtattgataaatcttgtaaatagtcaaggtaatatctatctagtgcatatcctgttatacgttgtctgtcttgagcatctacatctaaatagtgcgtcatctttttgtagttagcaaaagatatagataatatttgatttacaataggtttaacttttaaagcatcagtaacatctcttgaatctcgaaccattaaaaaagtatcatcaaataattggtgtaaattaacctcattgtgtaaatgattatagtacttatatagagtattagcaaatcttaagtaattaccttgctcaaatttatttaaagttagtaattttttataagtctgttttgtaagattgaaagcttcatgaactttccatttaggatttttaggtatatgaaatagtaatgcatttctttcaaataatccaaattcctctaagttatttattttgtcaatatttttaacaatatctgttaaagttgttaagtaattagaagttgaattttctcctataaaaatcctatatttgtctcctctataatttatatgaccataaacatctatattatccggacaccaactagaaaaatcaaaattatggtgttctaatgtttgttctattatctttattataacccctctatttaagttaggttgtgagtaattttttaaaataacatttaataaaacagataatgtcaattcatttttatattcacttttacttatatcatctttatacaaatttaaagttagttctttattaacaaggctatctgttaagaaaactttaacctcccctgttttaacatcaaatgaacttttattttctaaaacccatttattacccatattatgtttatctctgatatgtctaactttaagaccgaaagatgaattattctcagtacctggatgcatgtaccaagcactactatacaatgaatctgatatttccttataatacttacttgagcctttttctgtatcttcattcatataagaagtaatagatgattttattaaaccgtacttaccttggtctagaatatccataatatcatcatttaaactctctactacttccttatactcatctaattgtctagcttcatacccccataatcgggtttcattttctaattctttaattttttcttctacataacctttagattttatttgtcttctgctcttattaccatataaaggaaattcttttctttcttctctactggattcaatatattctttgtaatttcttcctttattttctccaattacaccttcaactaatttttcaactgtttcataagggttacctgtaaagtttgttacttctttattaccacatagtgctaagaataaatgtatttctgtagcagtatcaaaactaaatatattatgaatatctctaaataattccttagagcctaagttaattatattatttttcttcttcttaaggaatacatcttcttctcctatgtagatacatcctttattaactttaggtaaattaataatttcttgttctgttaatcctttttgtttatatgttattgccatttaaaatcactccttatttgttatgtactaatcatatcatagtaagtcatatttgtcaacaaaaaaaaagaagaactttttaaagttcttctaaatgagtttcgtatataaccttttgaattttatttagtggttctaaatctaaattcctaataagtttttcgtattttcttgaattcttgaagttaatagtattaggcatagcaagagcttcatcaacgtctttagtatagcttacaacatctgaatagatatctacttcttttacatatagaccctgagttaaacttctaaatactacctcattatgtgctataacttcttctttcttttctatgctcatttgtaaacctcctggtttattctacccaaacaagtacgtactctaagctagttaatgatactgatttaatattatttaattcttgcaatttcttaatatctacatcatagtttttacttatagtccataatgtctctcctgctcttactttatggtaatacttattcccctctttaataaggtcatccaatattacctacctccttgagtaataattagcctgtagataacatataagtataagaacaaagtttacaaattcagtagctataatatgaacataggtatgtgataaaaccatacttactatcaacgaagctaatcctaatccaataataaggaatagaaatctgtttgtcccttctgaacttttagttttataaaatgttgttatctgagttacatacgcaaggataatagtaatagttgctacagtttgtgttaaggctgtaaagtcacttaataaaaatagtaacaatgagaacacaataataaaaggtatagagaaataatccttttttctatatgaagctactaataagcaaacaatacctagagttaaattaagaccaactgatactacttgaaacattgtagcatcagttaatagtaaattgtaaaaactaatacctactgtagctacaattaaataccaaaaataactactaactcctttaacactatccgatttaactaaagctattagacctggtatataacctactgtaactaatatagcatataatatactcaagtaatgtgataaattatccatcttgttctcctaatttctctaatatttctaaaacttcttcccaagagataaatccttctccgtctgttaattccaaaaccataccataaacaaattgatttgtactaaattcagctctgtccgggtcgtcataaccttttccatgtccttgacgaatatccgagcaataaattaaaacaggtttatttacaatattttcaagtttttctacgattaattcctcttcttcacttaaccaagtttcaaatctatctgatatataattaaagtgttcttctccattatcatatatatggttaatagtttcttgtgcttgatgcttcatacctagtaaaataccaagttctgcaatcgttcctagcccctcattaagaatatcaaacacaaatatatctgagtcttgcatagctttaaagtcattgtttaaaatgcgctctgctaaaccagtttgttctgcattagccttatcattaattgatttatctttatgagggctgtaaggagtgactcctacaataccttctacttgcttatgctgtttgtctctataatctaccatagcttggttaagtagatgacctcccatataaattactttgtctttaatataattaaccatttatagtatctcctttttcttctagaatacctcttaaaatgtgtggcatctttttcttaatttgtttttctataattttcatcatattttctttttcttcttccataatatcatcaacaaaattttgacctacttgtttcataattaaaccaaaattttctaattctaaatcatcttcagacaatctgttttcttctatagctctaaaaatcattttttccattcttgattttgtgatagcataatctgccacagaatcattacttctaacttctgatttcattttcttacgactaaactctttaaattccttagatactaatttaaagtaatcatcatgttctgatttaccatctaaatatttaataacaataccttctcctgtattaggtttaacagtcatatctgattgtcctactaaatcttgaatttcttgaggatttaattcattaagataaaaagatggttctacaatcattaaagtttttacagttttcaaacctaatgtttctgataaagaaatgacttctgaatgaggtaaataggtttcactatccttatcgtatacatcgaatacataaaaatcattataacaatcttctttataatttaccttatgtttaactaaccattctccaaatataataataccttctaatatagataaatctaatttatctgtcatgttttcatgtacccaattataaaaaccgtttaatgtttcgttttcatttaatttttttctacgagagaaacagactaactcaccattctcggtagtaaaacttgcgttgcttccatctaatttttcttgaattacaaaacctctatctttaaatttgtctaaagataatcctttattttttactttagtataagatttcattaattagttatcctcctttgaattatgtactattgaaaataaaataagacttacacttgctaaaaatgctaataccactaaaccaggtaaatttagaactgttgataagaataatgatattgcacttataatataaactaaaccgcttagaaataaagttaataatacaattgttataagttttaccaaccaactgttattaataaataccttagctaaataattcataaaaaatcctccttagttattataaagtaagtatatcataactaaagaggtttgtcaacatattattttaccatttaaaattgtctgcgtactgagctaacttagaacggaaattaactgtaaaattatggaataccgcaccatcataatttttaaaatattccatataatctccaaaacctgatttactttcattttttaaatctatttgcttgaaatttccttcaactattactgtagaattttttgtatgaatccttgtaagtactttcttaagttcacttcgtttaaagttttgtgcttcatttataattatagtagcatcccttaaattaccgcctcttaaaaataaatgtgatatttgggatacccaacattcacctaatttatcttctttgacattatcttccatcattaacatttcggttatttgttgttcaggactcatattaagttcaataagggcatcatgtaatcccatgaaataagccatttctttttctgtttggttaccaggtctactacctaaatcttctgatacaggtgaaattacaaatactaattttctgttcttatttagataatcagcataagcacaagctactgaacacattgttttacccgtaccggcttgactttcattccaaagtatttcaacattatcattaaagaaatcctcacagaaatctaattgctcagttgtagctttttcaaggaattcattgaatactagatgttctcccatgttatatcttacattaggataatcctttaacttaaagtctaactcttttagttgtattgccatattttaaagttcccctatctataaatagttttactctcttttaatatagtactaattacctatatattcacctgttgaagaacaatagttactgcagttacattcaggataattttcacaaacttcttcatcttcatcatcaaataagtcttcatgataaatactagaatcaaaatctacaatacaattttcccctttacctttagatatgcctgtgtaaataacttcatcaaccatatcccaatcattacttactaagtaatttacagtatttgatactgaatcattatcaggtaagtagatatcaccatctgtaaacttaattaaaatatctccttgagggagtacatcattaattaaatcaatttctgtttcttcttcaattgtaaatacagttccttctagcttttcaggagtagtatgtgttaaatgtttaacatcttctcctgattcttcataaaatcctactgcattcatttccttgttatactttgcaataaatttaccattatcactgactaaatattgactgtttgcattataatcttctgcatcttctaaagtcatagatgggttatggtctttaacataataactaatttttctaacatctgttgtttgaattttctttccttcacctttaattactgaattaatttttttcataatattttctcctttttatatatcaattgatttttttgcaagattatcagcgtagtcattccatttatcatttgagtggctttttacttttacaaagtttatatctattactttttggtattctcgtatcatattaatatatgttttacttagaatatttcttgcagaccaagtaccttcataccaatgtattaaaccaatataatctatataaactattgcctgattgtatcctaattttatagcctcttcgataccataacaacaagctaatatttcacctgcaacattattatactttatcaatcctggtttgtcaacacttttactaatttccgctattatatttccttctttacttactaagacagcacctgagcctactttacctttattataagatgagctaccgtctgtgtatatatttacactatcctgcatatttataatcctccataaattgagggaattcacaatctgaatatacttctctgcagaaagatactgaaatataattaaaatcaaaacatttgaaacagtgttcttgaacttcttttttgtctttagcaatcacattaaatttaaaaccatcagctattactgtaaatactccttttttcataaaacaaatacctccactaattttattttaaattaataactaactcaataaacgatttaatagttttatttttaccttcatcaatatctgaaaagaaattaattaaactatcatcttcatcaaataaatcttcaacatcatcaaatttatttaatatatctgtaacactataaccttcttctgatatatactcatgcaagtcttctccatcctctgacagagttgcttctatcttaccattcttactttcaattaaatacaaagtatttaatactttaatagaatctactactacactgtaattactaatagtaggatactctgtataaagtatttccacattagtattcatataactatcaattacagaattaactgtgtccctttttaattcagatacattatgttttcgtatagtagggaattcttcgtcatattctactaattcttttctatctgtattcaataatttgtctaaagaagataataatactattttatattgattgtccggtagactatctgtaatttccataatggttaaaaaagtatcttcacctaaaactttatttatatcttgtaattcaaatgaatctaccatttcaatagtatcatctatatcatctgtagtcattaaaaaattaactaaattattattctccatcgtcttcctccaattctttaaatagctctttacctggagtatttaacgccttctctaacctcattaaattagcacttcttggtttctttttaccatactcccaataagatataagagaataatggactcctatctcagaagctaggcttcttaatgtatgtcctttttctactctaattttttgaagatttagaggtttactttcctttttttcatccataactatttctcctctacttttaaaaatttaaaatcctcagatgcttttgcattttttagtatatactcttgtgatttatttcttgcctctgctttacttttagcatataactctatatgaaatacatgaggtttttttaaagatggtgattcatatctccaataaactttaaaaagtagtgtttctttttttagaacattaattctaaaccatcttttaaatttattcattcattatcctcctttatttatttgttaaactaattatagcatagttaacttatgaagtcaactataatatacaaaaaagactaagaaattaatcttagtcttaatatattaataactattatgtgcgttgtggtatgcaagagctcctgatgttgaaccgtaacggtcaatcatatattgttttgcacctttagtttgttctgctatagaacccccactccatgatttacctaatccttggaatagtccttgagctcctgatgatgcattaacggcattaggattcagtgtagattcacgcatagcaatttcaatcattgcctcgtctccacctgcttgtctaatttgttctgctacagaacctcctgtagaactagttgattgtgtaggttgtttagtttctttttgaactggtgctgatgttggttgtacttcttttttagtatcttgtttattttgagtatcaaattgcgcttgttgttggtctactttttgttcaggtgtttgttcttctcctgctaatctagatactgtattatctacttgagttgaacctgagtgatattcgtatccaaaattaccattataattatagaaatgataagtaaattctccgtcactgaatgagaaatcatagttaccttcttgaattggttttgtatttacttctactgaatttgatttagcttgttctgctaacttattataatcaatttcgtctgcactagcttcgtttgtagcaatacctccaaaagtaatagccgtacctaatgctaatgttgcaaaaattgttttcttcataaatttaaaactccttaaataattttttagaattgtttatttgtaaatcgacataagtaatcataacatatatctttaaataacgcaagtataatatagcactaattagtgtaatattattaaagttttattacaaacattacagttatcagataattaaagacaaaaaaagagaggtattaacctccctaatttattatttccctgttacgtctacgatagttccgtctccgccaatttgaataggttgtttaccatcccatttttcaattaactgttgacgtaatatcttatcagataaagaagattctctaatctcattggcttttttatcaccattagcttctacttcttttttcttagcattttgttcagcaatttgtttatcaactttagtacgttctaactcttggttagctttaacacggctgtcaattgctttttgagtgttcttatctgctttagggctagataatgcaatatcctcaattacaaatccttgtttttctaagttatcatttaagctatctaaagtatcttttttaatttctcctgtcttaacaccaaatgcatcaattacagaatatttagatactgcctgacgaacattatcttgcacacgtgaacgcaagtatcctttttctagttcttcaatatctgcactaccaaaacgattaaataaatctacagctttagttgcatctactttataagatacatcaatatccatttgtaaatttttgccgtctgaagttgctacatttaaatctttatatttatgtgtttgtgttttagttggatatttatttaccttatcaaaaggtgctgttaagtgccaaccaggtgatttagtatcttccttaacaccatttactgagtatacaactccaacatgaccttgtggaatctttgtaatacacattaataaaataataaatcctataattgctaaaaaccctaatactcctgaaataactactgactttctcattacatttctcctttttctatttcttttattaagctatttaaagctttttcctcttggtctatttcttgcttatcggctctagttacaattgattgtctgcggtcgtttaagaattgttttttatactttacatattgttctaaaccgtattcatctaatgtaccttgcctaactaattccctgtattgttttcttatgttacttttcttttctttcattgaaagaaaatcgaatacataacttataccaaaacctacaaggactagaaaaacaataaaaatagcaaagtatgttaaaaataatgccatgtaattcctcctttatttgattacatatataactatacactatgtatttaattttgtcaacacttttttgcaaaaaaaaaatagacggattttaaatccgtctaaatttctattctatttgaatactccccaagcaaccccaggtatatgattaggtggaactccttgacaagttctaacagggcaatatactctgttaccattgtaagcattgtaacctatccaaatgtgacctgcttggatacaaacttcgtcatatacaattgtagctcctgccggtaagttacctcctactggagcatttaagaatggagaacctattctagttactataggttggttaccattaacaaatgttgcattttccggtttgtaccaagttccgtactggttctttttccaagagcctgttactggtctagttgccggtgtacttgcgctacttgttttaccgtctttaactactgtagaacttgaagttcctttatccatgtagtttttaatttgtttaatgaaataatcttttagtttattcattattgcttgagatggtcttccttgtgttactggattaaatcctgtatgaagaaccatagaacgatgaggacaagctgttggaacaaattccatatgcaatcttacagttttacgattaggagtaagaccccattctttaaatttctctgcagtaaattggaatactgcttgttcgtttttaaggaattgagcatcactagcactcattgattgacagacttcaatacctgcaaatctaaagttacctgagtttgctcctgttccatctcctgtgtgccaagcaatttgattcttagcatctattgcttcccatacataaccttcagagccatagtaatgagcaataccattagcatatctagcataacctgcgttagctaatgaattctcgtattgttgccctgaagaacgacctgcatcgttgtgtattaccattccttcaggtttcttaccacgtttatccattgtatagttaatatggttcttagaaacttttagtgttgctttctttttaggtgcaggtgttttacttgcacttttcttagctgtttcttttttaacagtagttcctgcttttacaggtatctcaataaaatgagttaatccgtaataattatctacacgttttgtaggttttttattagcgtaaccgttccagttttgctctaaaatagtaaatgtagaagtattacctccatcatatacaatacctatgtgaccccattgctgataactaccggatgtaaatacagcaatccatccttttttaggtactgtagaaggtttattttcatgtattttaaatccagtaccataactttgtttgatttggtctttagcattaccccaagttctaactttattatctgttaaccataatacataatcagtaattaagtcttggcactgtgcatgatagtaaccatctgcgtcaattgctcctgcttccattacaccaaacgatgggtcatagcttgtagcttttttaattctataaggactgtctactgtaccttttgcataagcgtctaaacgtttatttatttctgcttgagtcttagccattacttaacttcctcctctgcaaatactttaccgtgttcctcggtatcttcttcatcttgagaaggtgctgaaccgccatcaatttcatcttcaatagcaggtacttcatcactatcatctgtatcaggttctgcattgttttcatagttgtctaattcgaaagtgctagctgtgtttgcatttgcttgccattgaacaaattcattagggtctttactatcacgaggttttatataatctgtttgaacaatatcactatctttaagacctttagtattattatcaacaataatacctaaacctgctaataatgttagtatagaacctatgatatttacaccttgctcaatttgagctgagtagtctaaaccaaaagcacctgtaatttgattagcaaatactgctactgctgatataattgctacccaaaatgttttactcttagttcttgtgctaaagtttattcctccaacaactttaggttgtttagtttcattagccattaaaaaaccgacctttctattatatttatttctaacaataatataacagtaggtcggtcatgtttatctatattaatttaacacttactcattaatttgatttagttttttgataacttcagacatttgtttgttatctaaatcttctaatttagtttcaggtaataactctaatttatcccaaacttcttctttattagatactttattattaataattgccttaccaactaaactttccgtataatataattgctttgctgatgccatttgtatctctccttttaaatatgtaaagtatatagctagtatcgtatcctaggaacaaacacttgcgctatatactcaatgaaactctaccctcattcgaggacacagcaaaccgattcgtcaaccgcacatgtgaattctcaagtttcatttatgtaacacaccctctttgatttgcacaaagactaagggttttggagacccttgtactactaattatactaagggtgtttattatggtttctattggatttgaaccaatgacacctagagcttcaatctagtgctctaccatctgagctaagaaaccttgtaacgactcatacgagactcgaactcgtactctctgccgtgacagggcagtgtgttaaccagttacaccaatgagccaaaataaatgctataccctaaccttaccttaatgtatagcaggttttcatataagctcgaagcaacgattattaccactcataacaactatatattaagtgaaaggaggtgaaatgaacaaaacgtggtaattggtacttatataggaagtatgtataatctacaaggagtaagttattggttcataaaggagtgtgaacaataaatacatgaaagagtgaaagtctactccttgtagattcttttttaattatcaatcaaaggaggaaactgataattgttaataataaactataaagaggaaaatatttatagtcacattctgatataatgcaactaaatatccaagcataacccgtctcacgaggaacctacctataagacctgttattaagtgaatcactacgattgactctattaaggagctaccttaagtccatctcacgcaatttaaaagggacttacaaaccgtataacgatagtaagtttattaaataatgtgatattaacatattagttaataactttcacatggtcgaagaaaagtaaatttatttgatttccaaattatttttatcaaatatagcttcttttgaacctgtagatttatgctactcatactaataacctctattatctaacacatttctgtgctccaactacagttagtcgttacagcgtatctttctaggattccgctaagaccctagaaagaaattaaaccctagccgttatcatactctacagaccttataagtaagtaccaagtataccaatcgtatttaacaatactaatgacgacccatcctaccgatatatttccgataggttttgattcgtttgattatcttgtaccttatgactaccaaatcattattcagtcactatgctcagatatttagttgtattattatatattaattataacataatttttagtacttgtcaagttaattttaaaaaaaattatagaagtaagataaaattcttacttccaattaaaattataaggatgatacacattgttattgtttaaactggaaaacaatgtaagaaaaaacagtgatgtgtaaggtatttgttttattgttaattacattatagcatatactgatacctttgtcaagttaatttaatactttttttaaaatattagttatcttttgttagttcttcctgaatagcatcccatcttctttctgcttcactacgattatcttctatatgttttgtagttttacaacatttgatacagtatatatctttgatatgaccttcttctcttttatttgctctttttcttggtactttgaatacattaccacattctttacatattaaacttgagtaaaacattttttgtcttttcataattaatcaattccttttcttgttttatttgataatttaactatatactatattgataaataagtcaacagttttctaaaaataatttaaattattttgaagaatcctttaataccaagggttacaagagaaaaagtacgtatttagaaaataaggagtactcctattatatataattatattctgatataagtaattaataatattaaatatataattataattaataaggttgggaaaattgaattaaatataatctgatacttggtaaaactactcaatatagaagtaaaatcctttagtatcagtacttacagacaaaaaagtacgtatttagaaaataaggaactctcctattatagttatatatacttattaattattattaattactatctaaatatataattataattaataaggttagagagtcaacaaaaagacaaataaaaaagtgactacttaaagtcacttgttaatttaaaaaactattttaaatgattcaatcattcttgggttaatataaattaaactagaactagcatcttcagttgtattaacttttattgatgctgaatcagaaccactaatcatatagtctatatcttttctagtgtaacccttttcgtcagatgatactaaaaaatatttagcaccacttaacattgttatttcaatatgttttgacatctacaatctctcctatgcaaatttgttaaagacaaaggatagtatagcacctagaacgagtaaaagaaccttctcagtagtatccttcttcctagtatctttagtttttgtactttcagcaagttctgaaatcttttcatcaagtctttctaattggacgtaaattgctgattgtttttcactattgacagctacatctttatctatactaactatcatttttcttagttcagctacctcaacttctaaatctttgaaagttcctctatctatataattaccttcttgtatcttagacttaatagtttctacttgagaaacaaggttgtttatctccttatccaactagaatcacctctaaggtctaaccgtttcagattcagaatggatatcataattttctaagaaatcattgataatctccatataattatccgtaacgacttttccgtaagatgtttttgtatcaatttcaaacctaagcttaccaaaactttggaggtctaattcttttattacaatattagggtcatcagaaggaaggtaataatagtcgaagtatataattgagccatttattaatactctgtctattctatagacgtggaaatagcgtctgtctcttttaaaatgggctagtgcatctttaaactctaacttaaggatatccttatatttagtcaaagtggtaacctccttactattaatttttaaatttacttattttgtggtataatagttatgataaaggcagttattataattatattaagaataatgataataattattttttctgagaaaataagccaaatactaaaaacagataaagcatagatagctgatagatatactatattaagagttaccttacttttatcttctttatagatagaataacctaaagacgttgtaacaccactaagtataaaataatagaaacaaaaaagaggtatagacagaaaaaaagatacgataatcattgttaaacacctatttctttttgacctattatttatagaacttttagattacaccactaatataacattaaaagccagtcataaaagtcaattgttagattaataatataataaaaaaagacaataggaggttaaagtggttgaataataacatagctatattcatattcaaaacactggttatcattatattcttactactaattttgtctgttattaattccttgtcccttatttattcaataagaccgagtgtagttatgacatactttatatttggtggtattgtttctaatgtcgcacttactataacagataagttcttactgaagaaagaagaccccctacctgaatatgttcttaaaaaagtagagataaatgataaagaaataagaataatcaagaaaatcatagaaagtaactacggtataacagcagaagagataaaagttagggctaaagcacaaagaagaatagaggaagatagtaaaaaggaagattacgatgaaaacaaagaaagaaattaaagaacaaaggaaagagcttaaggatggtgctacatctgtttctttagtaaaaaaaggagataagagaatagctagccctagtagaatttgtagcctatgcggtcagcagttatcaggtatgaattacactaaaggaaaagcattatcaaaagttaatcattttcatttacagtattctaagtatatttattttgatatttgcgcagatatcaacaattgttataagaatttaagaaaaagaggtgaaatggattgagtgcagaaaatattagagatataattaacaagaaaaagttagaagaagaggatacaagaaaatatatagctgatggatttatgaatggtataggtaaattaatgtatgaattcaataaaaaagtagataataaagaaatagaagttaaagaccctaatgatttatataaactattcgtgatattctctcaaatgcaaaatatggttaatgaaacttctgaaggtggagcaatacctcagctatctagacctcagcaagaattatttgacgagattacaacagaagatagtaatggagaatctacagttgatttacagaagatacaagaaatgacagcagaagatattacagcaatgatttctgaaaaagaaaaagtaatgaatgaggaaaattcagaaacattctaaggagaaagatataa